(19) United States
(12) Patent Application Publication
(10) Pub. No.: US 2025/0268917 A1
(43) Pub. Date: Aug. 28, 2025

(54) PHARMACEUTICAL COMPOSITIONS AND METHODS FOR PREVENTION AND/OR TREATMENT OF INFLAMMATION

(71) Applicant: REVEN PHARMACEUTICALS INC., Westminster, CO (US)

(72) Inventors: Fatih M. UCKUN, White Bear Lake, MN (US); Michael A. VOLK, Broomfield, CO (US); Peter LANGE, Golden, CO (US); Brian D. DENOMME, Northville, MI (US); Hendrick Johanness Petrus VAN WYK, Glendale, CO (US)

(73) Assignee: REVEN PHARMACEUTICALS INC., Westminster, CO (US)

(21) Appl. No.: 18/029,529

(22) PCT Filed: Sep. 30, 2021
(86) PCT No.: PCT/US2021/052884

§ 371 Date: Mar. 30, 2023

Related U.S. Application Data
(60) Provisional application No. 63/085,745, filed on Sep. 30, 2020.

(51) Int. Cl. A61K 31/573; A61K 9/00; A61K 31/375; A61K 31/4415; A61K 31/455; A61K 31/51; A61K 31/525; A61K 31/714; A61K 33/06; A61P 31/14

(52) U.S. Cl. CPC A61K 31/573; A61K 9/0019; A61K 31/375; A61K 31/4415; A61K 31/455; A61K 31/51; A61K 31/525; A61K 31/714; A61K 33/06; A61P 31/14

(57) ABSTRACT

The disclosure relates to pharmaceutical compositions and methods for the prevention and/or treatment of inflammation, disease, and disorders. The treatment may include treatment of COVID-19.

### CROSS REFERENCE TO RELATED APPLICATIONS

This application is a 35 U.S.C. § 371 national stage application of PCT/US2021/052884, which was filed Sep. 30, 2021 and claims the benefit of U.S. provisional application No. 63/085,745, which was filed Sep. 30, 2020, titled PHARMACEUTICAL COMPOSITIONS AND METHODS FOR PREVENTION AND/OR TREATMENT OF INFLAMMATION, both of which are incorporated herein by reference as if fully set forth.

### BACKGROUND

A significant minority of patients infected with the new coronavirus, SARS-CoV-2, the causative agent of coronavirus disease 2019 (COVID-19), develop viral pneumonia that causes an acute lung injury (ALI) capable of rapid progression to viral sepsis and acute respiratory distress syndrome (ARDS) with a high fatality rate especially if they are older and have comorbidities. A systemic inflammatory response syndrome, also referred to as cytokine storm or cytokine release syndrome (CRS), contributes to the development of ARDS and often irreversible multi-organ dysfunction syndrome (MODS) associated with the severe-critical forms of COVID-19. Approximately 20% of COVID-19 patients with mild-moderate disease progress to severe-critical disease, and this percentage increases to 40% for the high-risk subgroup ≥65 years of age with comorbidities or laboratory parameters indicative of systemic inflammation, such as high levels of C-reactive protein (CRP), lactate dehydrogenase (LDH), and Ferritin or dysfunction of the coagulation system, as evidenced by elevated D-dimer levels. High-risk patients not only have a higher incidence of ARDS owing to severe viral sepsis caused by SARS-CoV-2, but they also progress faster and have a significantly higher case mortality rate. COVID-19 patients with an underlying cancer, especially if they are undergoing chemotherapy, are at an augmented risk for developing potentially fatal ARDS and multi-organ failure. Therefore, treatment platforms capable of preventing the disease progression and/or reducing the case mortality rate in such high-risk COVID-19 patients are urgently needed.

### SUMMARY

In an aspect, the invention relates to a pharmaceutical composition for intravenous delivery to a mammal. The pharmaceutical composition comprises magnesium sulfate, ascorbic acid, thiamine, and niacinamide at a ratio (w/w) of 72 to 108 magnesium sulfate: 80 to 120 ascorbic acid: 5.6 to 8.4 thiamine: 10.4 to 15.6 niacinamide. The pharmaceutical composition also comprises at least one anti-inflammatory drug, which is preferably dexamethasone.

In an aspect, the invention relates to a method of treating an inflammatory condition in a mammal. The method comprises administering to the mammal an effective amount of a pharmaceutical composition. The pharmaceutical composition comprises magnesium sulfate, ascorbic acid, thiamine, and niacinamide at a ratio (w/w) of 72 to 108 magnesium sulfate: 80 to 120 ascorbic acid: 5.6 to 8.4 thiamine: 10.4 to 15.6 niacinamide. Preferably, the administering further comprises administering one or more anti-inflammatory agent. The administering of the one or more anti-inflammatory drug may be conducted separately from the administration of the pharmaceutical composition. The pharmaceutical composition may comprise the one or more anti-inflammatory agent, and the administering of the pharmaceutical composition may be include administering of the one or more anti-inflammatory agent. Preferably, the one or more anti-inflammatory agent comprises one or more anti-inflammatory drug. Preferably, the one or more anti-inflammatory drug comprises dexamethasone.

In an aspect, the invention relates to a method of blocking the production and or release of the inflammatory cytokines in a mammal. The method comprises administering an effective amount of a pharmaceutical composition comprising magnesium sulfate, ascorbic acid, thiamine, and niacinamide at a ratio (w/w) of 72 to 108 magnesium sulfate: 80 to 120 ascorbic acid: 5.6 to 8.4 thiamine: 10.4 to 15.6 niacinamide to the mammal. Preferably, the administering further comprises administering one or more anti-inflammatory agent. The administering of the one or more anti-inflammatory drug may be conducted separately from the administration of the pharmaceutical composition. The pharmaceutical composition may comprise the one or more anti-inflammatory agent, and the administering of the pharmaceutical composition may be include administering of the one or more anti-inflammatory agent. Preferably, the one or more anti-inflammatory agent comprises one or more anti-inflammatory drug. Preferably, the one or more anti-inflammatory drug comprises dexamethasone.

In an aspect, the invention relates to a method of treating COVID-19. The method comprises administering to a COVID-19 patient an effective amount of a pharmaceutical composition. The pharmaceutical composition comprises magnesium sulfate, ascorbic acid, thiamine, and niacinamide at a ratio (w/w) of 72 to 108 magnesium sulfate: 80 to 120 ascorbic acid: 5.6 to 8.4 thiamine: 10.4 to 15.6 niacinamide. Preferably where the administering further comprises administering one or more anti-inflammatory agent. The administering of the one or more anti-inflammatory agent may be conducted separately from the administration of the pharmaceutical composition. The pharmaceutical composition may comprise the one or more anti-inflammatory agent, and the administering of the pharmaceutical composition may be include administering of the one or more anti-inflammatory agent. Preferably, the one or more anti-inflammatory agent comprises one or more anti-inflammatory drug. Preferably, the one or more anti-inflammatory drug comprises dexamethasone.

### BRIEF DESCRIPTION OF THE DRAWINGS

The following detailed description of preferred embodiments of the present invention will be better understood when read in conjunction with the appended drawings. For the purpose of illustrating the invention, particular embodiments are shown in the drawings. It is understood, however, that the invention is not limited to the precise arrangements and instrumentalities shown. In the drawings:

FIG. 1 illustrates In Vivo Protective Activity Of R J X In The LPS-GalN Challenged Mice in an Animal Model of Sepsis, Systemic Inflammation, Shock, and Multiorgan Failure.

FIGS. 2A, 2B, and 2C illustrate Effect Of Rejuveinix (RJX) On Serum Interleukin 6 (IL-6; FIG. 2A), Tumor Necrosis Factor Alpha (TNF-α; FIG. 2B), And Lactate Dehydrogenease (LDH; FIG. 2C) Levels In Lipopolysaccharide-Galactosamine (LPS-GalN) Chal-lenged Mice.

FIGS. 3A, 3B, 3C, 3D, 3E, and 3F illustrate Effect Of Rejuveinix (RJX) On Lung Vitamin C Levels, Protective Lung Anti-Oxidant Enzyme Levels, Lipid Peroxidation, And Histopathological Evaluations In Lipopolysaccharide-Galactosamine (LPS-GalN) Challenged Mice With Systemic Inflammation.

FIGS. 4A, 4B, 4C, and 4D illustrate FIG. 4. Rejuveinix (RJX) Prevents Acute Lung Injury and Inflammation in the LPS-GalN Mouse Model of Sepsis, Systemic Inflammation, Shock, ARDS and Multi-organ Failure.

FIGS. 5A, 5B, 5C, 5D, 5E, and 5F illustrate Effect Of Rejuveinix (RJX) On Liver Vitamin C (FIG. 5A), Malondialdehyde (MDA; FIG. 5B), Superoxide Dismutase (SOD; FIG. 5C), Catalase (CAT; FIG. 5D), Glutathione Peroxidase (GSHPx; FIG. 5E), and In Lipopolysaccharide-Galactosamine (LPS-GalN, FIG. 5F) Challenged Mice.

FIGS. 6A-6D illustrate Effect Of Rejuveinix (RJX) On Alanine Transaminase (ALT; FIG. 6A), Aspartate Transaminase (AST; FIG. 6B), Alkaline Phosphatase (ALP; FIG. 6C), And Total Bilirubin (FIG. 6D) In Lipopolysaccharide-Galactosamine (LPS-GalN) Challenged Mice.

FIGS. 7A-7D illustrate Heart Tissue-Level In Vivo Anti-Oxidant Activity of Rejuveinix (RJX) in the LPS-GalN Mouse Model of Sepsis, Systemic Inflammation, shock, ARDS and Multi-organ Failure.

FIG. 8 illustrates Effect Of Rejuveinix (RJX) On Serum cTni Level In LPS-GalN Mouse Model Of Sepsis, Systemic Inflammation, Shock, And Multi-Organ Failure.

FIGS. 9A-9D illustrate Effect Of Rejuveinix (RJX) On Brain Malondialdehyde (MDA; FIG. 9A), Superoxide Dismutase (SOD; FIG. 9B), Catalase (CAT; FIG. 9C), And; Glutathione Peroxidase (GSHPx; FIG. 9D) In Lipopolysaccharide-Galactosamine (LPS-GalN) Challenged Mice.

FIGS. 10A, 10B, and 10C illustrate Effect Of Rejuveinix (RJX) On Serum Interleukin-6 (IL-6; FIG. 10A), Tumor Necrosis Factor Alpha (TNF-α; FIG. 10B) And Lung Malondialdehyde (MDA; FIG. 10C) Mice Challenged With LPS-GalN.

FIG. 11 illustrates In Vivo Protective Activity of Delayed-Onset RJX Treatments in the LPS-GalN Model of Sepsis, Systemic inflammation, Shock, ARDS and Multiorgan Failure.

FIGS. 12A and 12B illustrate the effects of Rejuveinix (RJX) and the different doses of the Dexamethasone (DEX), treatments on serum interleukin 6 (IL-6;

FIG. 12A), tumor necrosis factor-alpha (TNF-α; FIG. 12B) in a Mouse Model of Fatal Cytokine Storm, Sepsis, Systemic Inflammation, ARDS and Multiorgan Failure.

FIG. 13 illustrates In Vivo Treatment Activity of Rejuveinix (RJX) and the different doses of the Dexamethasone (DEX) in the LPS-GalN Mouse Model of Fatal Cytokine Storm, Sepsis, Systemic Inflammation, ARDS and Multiorgan Failure.

FIGS. 14A and 14B illustrate Tissue-Level In Vivo Activity of Rejuveinix (RJX) and the different doses of the Dexamethasone (DEX), treatments on Lung and Liver Histopathological Scores in a Mouse Model of Fatal Cytokine Storm, Sepsis, Systemic Inflammation, ARDS and Multiorgan Failure.

FIGS. 15A, 15B, 15C, 15D, 15E, and 15F illustrate The Effects of Rejuveinix (RJX) and the different doses of the Dexamethasone (DEX), treatments on Acute Lung Injury and Inflammation in a Mouse Model of Fatal Cytokine Storm, Sepsis, Systemic Inflammation, ARDS and Multiorgan Failure.

FIGS. 16A, 16B, 16C, 16D, 16E, and 16F illustrate The Effects of Rejuveinix (RJX) and the different doses of the Dexamethasone (DEX), treatments on Liver Injury and Inflammation in a Mouse Model of Fatal Cytokine Storm, Sepsis, Systemic Inflammation, ARDS and Multiorgan Failure.

FIG. 17 illustrates Therapeutic Use of Low Dose RJX+Supratherapeutic High Dose DEX Combination After Onset of Systemic Inflammation and Lung Injury Improves the Survival Outcome in the LPS-GalN Mouse Model of Fatal Cytokine Storm and Sepsis.

FIGS. 18A, 18B, and 18C illustrate Therapeutic Use of Low Dose RJX Plus Supratherapeutic High Dose DEX Combination after Onset of Systemic Inflammation and Lung Injury Reverses Inflammatory Cytokine Response and Systemic Inflammation in the LPS-GalN Mouse Model of Fatal Cytokine Storm and Sepsis.

FIGS. 19A and 19B illustrate In Vivo Treatment Activity of Low Dose RJX, Supratherapeutic High Dose DEX and Their Combination on Lung and Liver Histopathological Scores in the LPS-GalN Mouse Model of Fatal Cytokine Storm and Sepsis.

FIGS. 20A, 20B, 20C, 20D, 20E, 20F, 20G, and 20H illustrate RJX plus DEX Combination Mitigates Acute Lung Injury and Inflammation in a Mouse Model of Fatal Cytokine Storm and Sepsis.

FIG. 21 illustrates In Vivo Treatment Activity of Low Dose Rejuveinix (RJX), Standard Dose Dexamethasone (DEX), and Their Combination in the LPS-GalN Mouse Model of Fatal Cytokine Storm, Sepsis, Systemic Inflammation, ARDS and Multiorgan Failure.

FIGS. 22A and 22B illustrate In Vivo Treatment Activity of Rejuveinix (RJX), Dexamethasone (DEX), and RJX+DEX on Lung and Liver Histopathological Scores in the LPS-GalN Mouse Model of Fatal Cytokine Storm, Sepsis, Systemic Inflammation, ARDS and Multiorgan Failure.

FIG. 23 illustrates the Effects of Rejuveinix (RJX) on Macroscopic Changes in Diabetic Wound Healing.

FIG. 24 illustrates the Effects of Rejuveinix (RJX) on Wound Area in Diabetic Wound Healing.

FIG. 25 illustrates the Effects of Rejuveinix (RJX) on Histopathological Score of Wounds in Diabetic Wound Healing.

### DETAILED DESCRIPTION

The words “right,” “left,” “top,” and “bottom” designate directions in the drawings to which reference is made. The words “a” and “one,” as used in the claims and in the corresponding portions of the specification, are defined as including one or more of the referenced item unless specifically stated otherwise. This terminology includes the words above specifically mentioned, derivatives thereof, and words of similar import. The phrase “at least one” followed by a list of two or more items, such as “A, B, or C” or “A, B, and C” means any individual one of A, B or C as well as any combination thereof.

A range preceded by a value and multiplication sign indicates that each value in the range is multiplied by the value. For example, 100×(0.7 to 0.9 mg/mL) means 70 to 90 mg/mL. As another example, 50 to 100×(0.7 to 0.9 mg/mL) means 35-70 to 45-90 mg/mL.

A range expressed as being between two numerical values, one as a low endpoint and the other as a high endpoint, includes the values between the low and high endpoints and also the values that are the low and high endpoints. Embodiments herein include subranges of a range herein, where the subrange includes a low and high endpoint of the subrange selected from any increment within the range selected from each single increment of the smallest significant figure, with the condition that the high endpoint of the subrange is higher than the low endpoint of the subrange.

Numerical values or ranges preceded by “about” refer to the explicitly recited numbers, and the numbers within the experimental error of the measure contemplated. Embodiments described with the modifier “about” may be altered to remove “about” in order to form further embodiments herein. Likewise, embodiments described without the modifier “about” may be altered to add “about” in order to form further embodiments herein.

Further embodiments herein include replacing one or more “including” or “comprising” in an embodiment with “consisting essentially of” or “consisting of.” “Including” and “comprising,” as used herein, are open ended, include the elements recited, and do not exclude the addition of one or more other element. “Consisting essentially of” means that addition of one or more element compared to what is recited is within the scope, but the addition does not materially affect the basic and novel characteristics of the combination of explicitly recited elements. “Consisting of” refers to the recited elements, but excludes any element, step, or ingredient not specified.

A compound in a composition or administered herein may be as stated, or a pharmaceutically acceptable salt thereof. A pharmaceutically acceptable salt may be an acid or base salt of the compound that is of sufficient purity and quality for use in a composition herein or administered in a method herein and are tolerated and sufficiently non-toxic to be used in a pharmaceutical preparation.

An embodiment comprises a pharmaceutical composition. The pharmaceutical composition may be for intravenous delivery to a mammal. The pharmaceutical composition may be for oral delivery to a mammal. The pharmaceutical composition may comprise magnesium sulfate, ascorbic acid, thiamine, and niacinamide. The magnesium sulfate, ascorbic acid, thiamine, and niacinamide may at a ratio (w/w) of 72 to 108 magnesium sulfate: 80 to 120 ascorbic acid: 5.6 to 8.4 thiamine: 10.4 to 15.6 niacinamide. The ratio may be 81 to 99 magnesium sulfate: 90 to 110 ascorbic acid: 6.3 to 7.7 thiamine: 11.7 to 14.3 niacinamide. The ratio may be 90:100:7:13; i.e., 90 magnesium sulfate: 100 ascorbic acid: 7 thiamine: 13 niacinamide. The ratio may be 90 (A to B) magnesium sulfate: 100 (A to B) ascorbic acid: 7 (A to B) thiamine: 13 (A to B) niacinamide, where A is less than or equal to B. A may be selected from 0.1, 0.2, 0.3, 0.4, 0.5, 0.6, 0.7, 0.8, 0.9, 1.0, or may be a value in a range between any two of the foregoing. B may be selected from 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0, or may be a value in a range between any two of the foregoing. For example, when A is 0.1 and B is 2.0, the ratio would be 0.9 to 180 magnesium sulfate: 10 to 200 ascorbic acid: 0.7 to 14 thiamine: 1.3 to 26 niacinamide.

The pharmaceutical composition may further comprise at least one of pyridoxin or riboflavin. The magnesium sulfate, ascorbic acid, thiamine, niacinamide, pyridoxin, and riboflavin may be at a ratio (w/w) of 72 to 108 magnesium sulfate: 80 to 120 ascorbic acid: 5.6 to 8.4 thiamine: 10.4 to 15.6 niacinamide: 10.4 to 15.6 pyridoxin: 0.24 to 0.36 riboflavin. The ratio may be 81 to 99 magnesium sulfate: 90 to 110 ascorbic acid: 6.3 to 7.7 thiamine: 11.7 to 14.3 niacinamide: 11.7 to 14.3 pyridoxin: 0.27 to 0.33 riboflavin. The ratio may be 90:100:7:13:13:0.3; i.e., 90 magnesium sulfate: 100 ascorbic acid: 7 thiamine: 13 niacinamide: 13 pyridoxin: 0.3 riboflavin. The ratio may be 90 (A to B) magnesium sulfate: 100 (A to B) ascorbic acid: 7 (A to B) thiamine: 13 (A to B) niacinamide: 13 (A to B) pyridoxin: 0.3(A to B) riboflavin, where A is less than or equal to B. A may be selected from 0.1, 0.2, 0.3, 0.4, 0.5, 0.6, 0.7, 0.8, 0.9, 1.0, or may be a value in a range between any two of the foregoing. B may be selected from 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0, or may be a value in a range between any two of the foregoing. For example, when A is 0.1 and B is 2.0, the ratio would be 0.9 to 180 magnesium sulfate: 10 to 200 ascorbic acid: 0.7 to 14 thiamine: 1.3 to 26 niacinamide: 1.3 to 26 pyridoxin: 0.03 to 0.6 riboflavin.

The concentration of magnesium sulfate in the pharmaceutical composition may be selected to fulfill one of the above-mentioned ratios. The magnesium sulfate may be at a concentration of 0.7 to 0.9 mg/mL. The magnesium sulfate may be at a concentration of 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 mg/mL, or at a concentration in a range between any two of the foregoing.

The concentration of ascorbic acid in the pharmaceutical composition may be selected to fulfill one of the above-mentioned ratios. The ascorbic acid may be at a concentration of 0.8 to 1.0 mg/mL. The ascorbic acid may be at a concentration of 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 mg/mL, or at a concentration in a range between any two of the foregoing.

The concentration of thiamine in the pharmaceutical composition may be selected to fulfill one of the above-mentioned ratios. The thiamine may be at a concentration of 0.05 to 0.07 mg/mL. The thiamine may be at a concentration of 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, or 0.2 mg/mL, or at a concentration in a range between any two of the foregoing.

The concentration of niacinamide in the pharmaceutical composition may be selected to fulfill one of the above-mentioned ratios. The niacinamide may be at a concentration of 0.105 to 0.150 mg/mL. The niacinamide may be at a concentration of 0.095, 0.100, 0.105, 0.110, 0.115, 0.120, 0.125, 0.130, 0.135, 0.140, 0.145, 0.150, 0.155, 0.160 mg/ml, or at a concentration in a range between any two of the foregoing.

The concentration of pyridoxin in the pharmaceutical composition may be selected to fulfill one of the above-mentioned ratios. The pyridoxin may be at a concentration of 0.105 to 0.150 mg/mL. The pyridoxin may be at a concentration of 0.095, 0.100, 0.105, 0.110, 0.115, 0.120, 0.125, 0.130, 0.135, 0.140, 0.145, 0.150, 0.155, 0.160 mg/ml, or at a concentration in a range between any two of the foregoing.

The concentration of riboflavin in the pharmaceutical composition may be selected to fulfill one of the above-mentioned ratios. The riboflavin may be at a concentration of 0.002 to 0.003 mg/mL. The riboflavin may be at a concentration of 0.001, 0.002, 0003, 0.004, 0.005, or 0.006 mg/mL, or at a concentration in a range between any two of the foregoing.

The pharmaceutical composition may further comprise cyanocobalamin. The concentration of cyanocobalamin may be 0.0015 to 0.0030 mg/mL. The concentration of cyanocobalamin may be 0.0005, 0.0010, 0.0015, 0.0020, 0.0025, 0.0030, 0.0035, or 0.0040, or at a concentration in a range between any two of the foregoing.

The pharmaceutical composition may further comprise a buffering agent. Non-limiting examples of the buffering agent are sodium bicarbonate, lactate, acetate, gluconate or maleate. The pharmaceutical composition with buffering agent may have a pH of 7.35-7.45. The pH may be 7.35, 7.36, 7.37, 7.38, 7.39, 7.40, 7.41, 7.42, 7.43, 7.44, or 7.45, or a pH in a range between any two of the foregoing.

The pharmaceutical composition may further comprise a diluent. Non-limiting examples of the diluent are normal saline, water for injection, or an intravenous solution, preferably commonly used intravenous solution.

The pharmaceutical composition may further comprise at least one of an antioxidant or an anti-inflammatory agent, which may be an anti-inflammatory drug. The at least one of an antioxidant or an anti-inflammatory agent may be one or more selected from a Cox-2 inhibitor, a Cox-1 inhibitors, steroids, zinc, copper, selenium, Vitamin E, and Vitamin A. The concentration for each antioxidant or an anti-inflammatory agent may be 1 nM to 100 μM. The concentration for each antioxidant or an anti-inflammatory agent may be independently selected from 1 nM, 10, nM, 20, nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 10 μM, 20 μM, 30 μM, 40 μM, 50 μM, 60 μM, 70 μM, 80 μM, 90 μM, or 100 μM, or in range between any two of the foregoing.

The pharmaceutical composition may comprise magnesium sulfate, ascorbic acid, thiamine, and niacinamide and at least one or more of pyridoxin, riboflavin, cyanocobalamin, a buffering agent, a diluent, an antioxidant, an anti-inflammatory agent, which may be an anti-inflammatory drug, a Cox-2 inhibitor, a Cox-1 inhibitor, a steroid, zinc, copper, selenium, Vitamin E, or Vitamin A. The ratios of these constituents may be as set for the above. The concentrations of these constituents may be as set forth above. The species of each generic constituent may be as set forth above.

The pharmaceutical composition may further comprise one or more anti-inflammatory drug selected from anti-inflammatory steroids. The one or more anti-inflammatory steroids may be selected from cortisone, hydrocortisone, methylprednisolone, prednisolone, prednisone, triamcinolone, betamethasone, fludrocortisone or dexamethasone, or pharmaceutically acceptable salts thereof. The one or more anti-inflammatory drug may comprise dexamethasone. The dexamethasone may be at a concentration in the pharmaceutical composition such that a dose of 0.75-40 mg or 1 mg-40 mg is delivered per administration. The concentration may be such as to deliver a dose of 0.75, 1, 2, 3, 4, 5, 6, 7, 8 9 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 mg of dexamethasone in one administration, or a dose between any two of the foregoing. See below for exemplary, non-limiting, volumes of pharmaceutical composition that may be delivered in one dose. One dose of the pharmaceutical composition may comprise an amount of the dexamethasone corresponding one of the foregoing dexamethasone doses. A non-limiting guideline for selecting a dexamethasone dose, and from that a concentration for the volume of pharmaceutical composition delivered in one dose, follows. When used alone, a dose of the dexamethasone may be 1-2 mg per administration, 4-8 mg per administration, or 10-20 mg per administration to treat mild, moderate, or severe inflammation, respectively. The frequency of administration may vary from 1-3 times per day. When in the combination of the pharmaceutical composition, the dose of the dexamethasone designed for treating mild or moderate inflammation (as a non-limiting example, 2-4 mg instead of 10-20 mg per dose) may be sufficient to treat severe inflammation, and the concentration of dexamethasone in the pharmaceutical composition may be adjusted accordingly.

The following Conversion Table provides a guide to determining a dose, and thereby the concentration in a volume of pharmaceutical composition to be delivered, for the cortisone, hydrocortisone, methylprednisolone, prednisolone, prednisone, triamcinolone, betamethasone, or fludrocortisone based on the above described doses for dexamethasone. One dose of the pharmaceutical composition may comprise an amount of one or more of cortisone, hydrocortisone, methylprednisolone, prednisolone, prednisone, triamcinolone, betamethasone, fludricortisone or dexamethasone to be about an equivalent to a dose of dexamethasone as described herein.

Conversion Table

| Anti-inflammatory | Approximate Equivalent | Anticipated |
|---|---|---|
| Steroid | Dose (mg) | Half-life (Hr.) |
| Cortisone | 25 | 8-12 |
| Hydrocortisone | 20 | 8-12 |
| Methylprednisolone | 4 | 18-36 |
| Prednisolone | 5 | 18-36 |
| Prednisone | 5 | 18-36 |
| Triamcinolone | 4 | 18-36 |
| Betamethasone | 0.6-0.75 | 36-54 |
| Dexamethasone | 0.75 | 36-54 |
| Fludrocortisone | 0.1 | 12-26 |

A dose for one of the other anti-inflammatory steroid may be as set forth above for dexamethasone. Or it may by adjusted based on a selected dexamethasone dose and using the above Conversion Table where the dose of the other anti-inflammatory steroid is calculated by [(approximate equivalent dose of the other anti-inflammatory steroid)/0.75]×(selected dexamethasone dose). The concentration in the pharmaceutical composition may then be arrived at by using the volume of pharmaceutical composition per administration. See below for exemplary, non-limiting volumes that may be used.

As shown in the Conversion Table, the half-life of the different steroids varies and this gives rise to a different duration of action. 8-12 hour half life is considered short-acting, 18-36 hour half-life is considered intermediate-acting, and 36-54 hour half-life is considered long-acting. One guideline to choosing one anti-inflammatory steroid, or a combination of two or more, may be the duration of action for each anti-inflammatory steroid. For example, a combination of more than one anti-inflammatory steroid may include two or three different durations of action.

An embodiment comprises a pre-formulation of any pharmaceutical composition herein. The pre-formulation may be 50-100 fold more concentrated than the pharmaceutical composition. The concentrate may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fold more concentrated, or a fold more concentrated in a range between any two of the foregoing, compared to a pharmaceutical composition herein. The concentration of a constituent of the pre-formulation may be arrived at by selecting the constituent from the above, selecting one of the exemplary concentrations for the constituent, and multiplying by the fold more concentrated value selected. The magnesium sulfate may be at a concentration of 50 to 100×(0.7 to 0.9 mg/mL). The ascorbic acid may be at a concentration of 50 to 100×(0.8 to 1.0 mg/mL). The thiamine may be at a concentration of 50 to 100×(0.05 to 0.07 mg/mL). The niacinamide may be at a concentration of 50 to 100×(0.105 to 0.150 mg/mL). When present, the pyridoxin may be at a concentration of 50 to 100×(0.105 to 0.150 mg/mL). When present, the riboflavin may at a concentration of 50 to 100×(0.002 to 0.003 mg/mL). When present, the cyanocobalamin may be at a concentration of 50 to 100×(0.0015 to 0.0030 mg/mL).

The pharmaceutical composition may be formulated for intravenous infusion, injection, subcutaneous injection, intraarterial injection, inhalation (i.e., as an inhalant), or nasal spraying (i.e., as a nasal spray).

An embodiment comprises a method of treating an inflammatory condition in a mammal. The method may comprise administering a pharmaceutical composition herein to the mammal. The pharmaceutical composition may be any described herein. The mammal may have an inflammatory condition. The mammal may be a human, dog, cat, or horse.

The administering may be intravenous infusion, injection, subcutaneous injection, intraarterial injection, inhalation, or nasal spraying. The administering may be intravenous infusion of the pharmaceutical composition. The administering may comprise daily intravenous infusions for a number of cycles, where each cycle is for a set of days (or one day), and a cycle may be separated from another cycle by 0 or more days. The administering may comprise daily intravenous infusions for 1-12 cycles of 7-28 consecutive days, wherein 0-365 days separates each cycle. The number of cycles may be 1, 2, 3, 4, 5, 6, 7, 8, 9, 10, 11, 12, 13, 14, 15, 16, 17, 18, 19, or 20, or a range between any two of the foregoing. The number of days in a cycle may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35, or a range between any two of the foregoing. The number of days between one cycle and the next may be 0, 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 365, 370, 380, 390, or 400 days, or in a range of days between any two of the foregoing. The number of days between one cycle and the next may be any integer selected from 0-365, or in a range between any two integers selected from 0-365. The number of days between each cycle may be the same. The number of days between one cycle and the next may be different than the number of days between another set of two consecutive cycles.

A daily intravenous infusion may have a dose of 0.025 mL/kg to 2.5 mL/kg of the pharmaceutical composition. The dose may be 0.005, 0.010, 0.015, 0.020, 0.025, 0.030, 0.035, 0.040, 0.045, 0.050, 0.055, 0.060, 0.065, 0.070, 0.075, 0.080, 0.085, 0.090, 0.095, 0.100, 0.110, 0.120, 0.130, 0.140, 0.150, 0.160, 0.170, 0.180, 0.190, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mL/kg, or in a range between any two of the foreogoing. The dose may be any 0.001 increment from 0.025 to 2.5, or in a range between any two 0.001 increments from 0.025 to 2.5. The daily infusion may be administered over 15-60 minutes. The daily infusion may be administered over 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 minutes, or in a time between any two of the foregoing.

The administering may comprise a dose of 2.5 mL/kg of the pharmaceutical composition. The dose may be 0.005, 0.010, 0.015, 0.020, 0.025, 0.030, 0.035, 0.040, 0.045, 0.050, 0.055, 0.060, 0.065, 0.070, 0.075, 0.080, 0.085, 0.090, 0.095, 0.100, 0.110, 0.120, 0.130, 0.140, 0.150, 0.160, 0.170, 0.180, 0.190, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mL/kg of the pharmaceutical composition. The administering may comprise a dose of 100 ml of the pharmaceutical composition.

The inflammatory condition may be one affecting at least one of the joints, skin, skeletal muscle, blood vessels, liver, gall bladder, lungs, heart, brain, meninges, gastrointestinal system, urinary bladder, urethra, or kidneys. The inflammatory condition affecting the joints may be arthritis. The inflammatory condition affecting the skin may be dermatitis. The inflammatory condition affecting skeletal muscle may be myositis. The inflammatory condition affecting blood vessels may be vasculitis, vascular leak syndrome, capillary leak syndrome, or retinitis. The inflammatory condition affecting the liver may be hepatitis. The inflammatory condition affecting the gall bladder may be cholecystitis. The inflammatory condition affecting the lungs may be pneumonitis. The inflammatory condition affecting the heart may be myocarditis, pericarditis, or endocarditis. The inflammatory condition affecting the brain may be encephalitis. The inflammatory condition affecting the meninges may be meningitis. The inflammatory condition affecting the gastrointestinal system may be gastritis, colitis, enteritis, or esophagitis. The inflammatory condition affecting the urinary bladder may be cystitis. The inflammatory condition affecting the urethra may be urethritis. The inflammatory condition affecting the kidneys may be nephritis.

The inflammatory condition may be systemic inflammation. The systemic inflammation may comprise at least one of sepsis, cytokine release syndrome, cytokine storm, graft versus host disease, or a multi-organ auto-immune disease. Non-limiting examples of the multi-organ auto-immune disease include Lupus/SLE.

The inflammatory condition may be caused by at least one of a toxic agent, radiation, an infection, obesity-related complications, autoimmune disease, bone marrow transplantation, organ transplantation, treatment with monoclonal antibodies, treatment with antibody-drug conjugates, treatment with bidirectional T-cell engagers, treatment with another biologic group(s) (biologic(s)), cancer, or cancer therapy.

Non-limiting examples of the toxic agent include alcohol, a chemotherapy drug, a poison, a controlled drug substance, and a chemical or biological warfare agent. Non-limiting examples of the radiation include sunburn/UV radiation, ionizing radiation from an irradiator and a radioisotope. Non-limiting examples of the infection include SARS-CoV-2 infection, viral infection, bacterial infection, and fungal infection. Non-limiting examples of the obesity-related complications include metabolic syndrome. Non-limiting examples of the other biologic groups (biologics) include recombinant therapeutic proteins, vaccines, and vaccine-like products.

The mammal may be a human. The mammal may have Ulcerative colitis, Crohn disease, Rheumatoid arthritis, hemophagocytic lymphohistiocytosis, chronic inflammatory demyelinating polyneuropathy, multiple sclerosis, sarcoidosis, rhematic fever, Behcet disease, Mediterranean fever, inflammatory pelvic disease, interstitial cystitis, or Heliobacter pylori. The inflammatory condition may be any of the foregoing.

The mammal may be a human person affected by an inflammatory condition (aka a “patient”), and the treatment may be intravenous infusion of a pharmaceutical composition herein.

The mammal may be a human and the inflammatory condition may be caused by infection of the human by the SARS-CoV-2 virus, COVID-19 in the human, or presence of SARS-CoV-2 virus spike protein in the human.

An embodiment comprises treating a mammal in need thereof by administering a pharmaceutical composition herein to the mammal. The mammal may have at least one of (1) viral sepsis, cytokine storm, cytokine release syndrome, pneumonia, Kawasaki disease, or ARDS caused by COVID-19, (2) bacterial sepsis, (3) fungal sepsis, (4) acute graft versus host disease, (5) fulminant hepatitis, (6) radiation pneumonitis, (7) acute flare of an inflammatory bowel disease (for example, ulcerative colitis or Crohn disease), or (8) a multi-system inflammation. The need may be to treat one or more of these conditions. The mammal may be affected by an inflammatory condition. The mammal may be a human, dog, cat, or horse. The pharmaceutical composition may be administered at any dose herein. The pharmaceutical composition may be administered to the mammal at 2.5 mL/kg or a fixed dose of 100 mL.

An embodiment comprises a method of blocking the production and or release of the inflammatory cytokines in a mammal. The mammal may be a human, dog, cat, or horse. The method may comprise administering a pharmaceutical composition herein the mammal. The dose may be any herein. The inflammatory cytokine may be Tumor necrosis factor alpha (TNF-a), interleukin-6 (IL-6), or transforming growth factor beta (TGF-beta).

An embodiment comprises a method of preventing or treating a human or animal disease by inhibiting production or release of an inflammatory Cytokine. The method may comprise administering the pharmaceutical composition herein to the human or animal. The human or animal may be a human, dog, cat, or horse. The human or animal may be affected by an inflammatory condition.

An embodiment comprises a method of treating or reducing oxidative stress in cells and/or tissue by administering a pharmaceutical composition herein to the cells and/or tissue, preferably to a mammal in need thereof. The cells and/or tissue may be mammalian. The mammal may be a human, dog, cat, or horse. The mammal may be affected by an inflammatory condition. The mammal may have cells and/or tissue affected by oxidative stress. The oxidative stress may be treated or reduced by preventing peroxidation of membranes. The oxidative stress may be treated or reduced by increasing the levels of anti-oxidant enzymes. The anti-oxidant enzymes may be one or more selected from catalase, superoxide dismutase and glutathione peroxidase. The oxidative stress may be treated or reduced by increasing the blood and tissue levels of ascorbic acid and niacinamide and thiamine. The method may comprise administering other anti-oxidants including but not limited to isoflavones (non-liming examples include genistein and daidzein); Vitamins (as a non-limiting example, Vitamin E), each may be at doses that yield 1 nM to 100 μM concentrations in the serum, or at doses that yield a concentration of each at a value independently selected from 1 nM, 10, nM, 20, nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 10 μM, 20 μM, 30 μM, 40 μM, 50 μM, 60 μM, 70 μM, 80 μM, 90 μM, or 100 μM, or in range between any two of the foregoing. The other anti-oxidants may be administered as part of a formulation including the pharmaceutical composition, or separately from the pharmaceutical composition.

Embodiments comprise the use of pharmaceutical composition herein to achieve any of the affects of a method herein. The use may be to treat an inflammatory condition. The inflammatory condition may be as described above. The use may be for blocking the production and or release of the inflammatory cytokines. The inflammatory cytokine may be Tumor necrosis factor alpha (TNF-a), interleukin-6 (IL-6), or transforming growth factor beta (TGF-beta). The use may be for preventing or treating a human or animal disease by inhibiting production or release of an inflammatory Cytokine. The use may be for treating or reducing oxidative stress in cells and/or tissue.

Embodiments may be use of a pharmaceutical composition herein to prepare a medicament for the treatment of any of the ailments listed herein. The ailment may be an inflammatory condition. The inflammatory condition may be as described above. The ailment may be one treatable by blocking the production and or release of the inflammatory cytokines. The inflammatory cytokine may be Tumor necrosis factor alpha (TNF-α), interleukin-6 (IL-6), or transforming growth factor beta (TGF-beta). The ailment may be oxidative stress in cells and/or tissue.

The pharmaceutical composition for a method or use herein may be for intravenous delivery to a mammal. The pharmaceutical composition may comprise magnesium sulfate, ascorbic acid, thiamine, and niacinamide. The magnesium sulfate, ascorbic acid, thiamine, and niacinamide may at a ratio (w/w) of 72 to 108 magnesium sulfate: 80 to 120 ascorbic acid: 5.6 to 8.4 thiamine: 10.4 to 15.6 niacinamide. The ratio may be 81 to 99 magnesium sulfate: 90 to 110 ascorbic acid: 6.3 to 7.7 thiamine: 11.7 to 14.3 niacinamide. The ratio may be 90:100:7:13; i.e., 90 magnesium sulfate: 100 ascorbic acid: 7 thiamine: 13 niacinamide. The ratio may be 90(A to B) magnesium sulfate: 100(A to B) ascorbic acid: 7(A to B) thiamine: 13(A to B) niacinamide, where A is less than or equal to B. A may be selected from 0.1, 0.2, 0.3, 0.4, 0.5, 0.6, 0.7, 0.8, 0.9, 1.0, or may be a value in a range between any two of the foregoing. B may be selected from 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0, or may be a value in a range between any two of the foregoing. For example, when A is 0.1 and B is 2.0, the ratio would be 0.9 to 180 magnesium sulfate: 10 to 200 ascorbic acid: 0.7 to 14 thiamine: 1.3 to 26 niacinamide.

The pharmaceutical composition for a method or use herein may further comprise at least one of pyridoxin or riboflavin. The magnesium sulfate, ascorbic acid, thiamine, niacinamide, pyridoxin, and riboflavin may be at a ratio (w/w) of 72 to 108 magnesium sulfate: 80 to 120 ascorbic acid: 5.6 to 8.4 thiamine: 10.4 to 15.6 niacinamide: 10.4 to 15.6 pyridoxin: 0.24 to 0.36 riboflavin. The ratio may be 81 to 99 magnesium sulfate: 90 to 110 ascorbic acid: 6.3 to 7.7 thiamine: 11.7 to 14.3 niacinamide: 11.7 to 14.3 pyridoxin: 0.27 to 0.33 riboflavin. The ratio may be 90:100:7:13:13:0.3; i.e., 90 magnesium sulfate: 100 ascorbic acid: 7 thiamine: 13 niacinamide: 13 pyridoxin: 0.3 riboflavin. The ratio may be 90(A to B) magnesium sulfate: 100(A to B) ascorbic acid: 7(A to B) thiamine: 13(A to B) niacinamide: 13(A to B) pyridoxin: 0.3(A to B) riboflavin, where A is less than or equal to B. A may be selected from 0.1, 0.2, 0.3, 0.4, 0.5, 0.6, 0.7, 0.8, 0.9, 1.0, or may be a value in a range between any two of the foregoing. B may be selected from 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0, or may be a value in a range between any two of the foregoing. For example, when A is 0.1 and B is 2.0, the ratio would be 0.9 to 180 magnesium sulfate: 10 to 200 ascorbic acid: 0.7 to 14 thiamine: 1.3 to 26 niacinamide: 1.3 to 26 pyridoxin: 0.03 to 0.6 riboflavin.

The concentration of magnesium sulfate in the pharmaceutical composition for a method or use herein may be selected to fulfill one of the above-mentioned ratios. The magnesium sulfate may be at a concentration of 0.7 to 0.9 mg/mL. The magnesium sulfate may be at a concentration of 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 mg/mL, or at a concentration in a range between any two of the foregoing.

The concentration of ascorbic acid in the pharmaceutical composition for a method or use herein may be selected to fulfill one of the above-mentioned ratios. The ascorbic acid may be at a concentration of 0.8 to 1.0 mg/mL. The ascorbic acid may be at a concentration of 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 mg/mL, or at a concentration in a range between any two of the foregoing.

The concentration of thiamine in the pharmaceutical composition for a method or use herein may be selected to fulfill one of the above-mentioned ratios. The thiamine may be at a concentration of 0.05 to 0.07 mg/mL. The thiamine may be at a concentration of 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, or 0.2 mg/mL, or at a concentration in a range between any two of the foregoing.

The concentration of niacinamide in the pharmaceutical composition for a method or use herein may be selected to fulfill one of the above-mentioned ratios. The niacinamide may at a concentration of 0.105 to 0.150 mg/mL. The niacinamide may at a concentration of 0.095, 0.100, 0.105, 0.110, 0.115, 0.120, 0.125, 0.130, 0.135, 0.140, 0.145, 0.150, 0.155, 0.160 mg/ml, or at a concentration in a range between any two of the foregoing.

The concentration of pyridoxin in the pharmaceutical composition for a method or use herein may be selected to fulfill one of the above-mentioned ratios. The pyridoxin may be at a concentration of 0.105 to 0.150 mg/mL. The pyridoxin may be at a concentration of 0.095, 0.100, 0.105, 0.110, 0.115, 0.120, 0.125, 0.130, 0.135, 0.140, 0.145, 0.150, 0.155, 0.160 mg/ml, or at a concentration in a range between any two of the foregoing.

The concentration of riboflavin in the pharmaceutical composition for a method or use herein may be selected to fulfill one of the above-mentioned ratios. The riboflavin may be at a concentration of 0.002 to 0.003 mg/mL. The riboflavin may be at a concentration of 0.001, 0.002, 0003, 0.004, 0.005, or 0.006 mg/mL, or at a concentration in a range between any two of the foregoing.

The pharmaceutical composition for a method or use herein may further comprise cyanocobalamin. The concentration of cyanocobalamin may be 0.0015 to 0.0030 mg/mL. The concentration of cyanocobalamin may be 0.0005, 0.0010, 0.0015, 0.0020, 0.0025, 0.0030, 0.0035, or 0.0040, or at a concentration in a range between any two of the foregoing.

The pharmaceutical composition for a method or use herein may further comprise a buffering agent. Non-limiting examples of the buffering agent are sodium bicarbonate, lactate, acetate, gluconate or maleate. The pharmaceutical composition with buffering agent may have a pH of 7.35-7.45. The pH may be 7.35, 7.36, 7.37, 7.38, 7.39, 7.40, 7.41, 7.42, 7.43, 7.44, or 7.45, or a pH in a range between any two of the foregoing.

The pharmaceutical composition for a method or use herein may further comprise a diluent. Non-limiting examples of the diluent are normal saline, water for injection, or an intravenous solution, preferably commonly used intravenous solution.

The pharmaceutical composition for a method or use herein may further comprise at least one of an antioxidant or an anti-inflammatory agent, which may be an anti-inflammatory drug. The at least one of an antioxidant or an anti-inflammatory agent may be one or more selected from a Cox-2 inhibitor or a Cox-1 inhibitors steroids, zinc, copper, selenium, Vitamin E, and Vitamin A. The concentration for each antioxidant or an anti-inflammatory agent may be 1 nM to 100 μM. The concentration for each antioxidant or an anti-inflammatory agent may be independently selected from 1 nM, 10, nM, 20, nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 10 μM, 20 μM, 30 μM, 40 μM, 50 μM, 60 μM, 70 μM, 80 μM, 90 μM, or 100 μM, or in range between any two of the foregoing.

The pharmaceutical composition for a method or use herein may comprise magnesium sulfate, ascorbic acid, thiamine, and niacinamide and at least one or more of pyridoxin, riboflavin, cyanocobalamin, a buffering agent, a diluent, an antioxidant, an anti-inflammatory agent, which may be an anti-inflammatory drug, a Cox-2 inhibitor, a Cox-1 inhibitor, a steroids, zinc, copper, selenium, Vitamin E, or Vitamin A. The ratios of these constituents may be as set for the above. The concentrations of these constituents may be as set forth above. The exemplary species of each generic constituent may be as set forth above.

The pharmaceutical composition for a method or use herein may be formulated for intravenous infusion, injection, subcutaneous injection, intraarterial injection, inhalation (i.e., as an inhalant), or nasal spraying (i.e., as a nasal spray).

The pharmaceutical composition for a method or use herein may further comprise one or more anti-inflammatory drug. Or the method may further comprise separately administering one or more anti-inflammatory drug. The one or more anti-inflammatory drug is selected from anti-inflammatory steroids. The one or more anti-inflammatory steroids may be selected from cortisone, hydrocortisone, methylprednisolone, prednisolone, prednisone, triamcinolone, betamethasone, fludrocortisone, or dexamethasone, or a pharmaceutically acceptable salt thereof. The one or more anti-inflammatory drug may comprise dexamethasone. The dexamethasone may be administered at a dose of 0.75-40 mg or 1 mg-40 mg is delivered per administration. The dose of dexamethasone may be 0.75, 1, 2, 3, 4, 5, 6, 7, 8 9 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 mg in one administration, or a dose between any two of the foregoing. A non-limiting guideline for selecting a dexamethasone dose is as follows. When used alone, a dose of the dexamethasone may be 1-2 mg per administration, 4-8 mg per administration, or 10-20 mg per administration to treat mild, moderate, or severe inflammation, respectively. The frequency of administration may vary from 1-3 times per day. When administered in combination with the pharmaceutical composition, the dose of the dexamethasone designed for treating mild or moderate inflammation (as a non-limiting example, 2-4 mg instead of 10-20 mg per dose) may be sufficient to treat severe inflammation, and the dose of dexamethasone may be adjusted accordingly.

The above Conversion Table provides a guide to determining a dose for the cortisone, hydrocortisone, methylprednisolone, prednisolone, prednisone, triamcinolone, betamethasone, or fludrocortisone based on the above described doses for dexamethasone:

Conversion Table

| Anti-inflammatory | Approximate Equivalent | Anticipated |
|---|---|---|
| Steroid | Dose (mg) | Half-life (Hr.) |
| Cortisone | 25 | 8-12 |
| Hydrocortisone | 20 | 8-12 |
| Methylprednisolone | 4 | 18-36 |
| Prednisolone | 5 | 18-36 |
| Prednisone | 5 | 18-36 |
| Triamcinolone | 4 | 18-36 |
| Betamethasone | 0.6-0.75 | 36-54 |
| Dexamethasone | 0.75 | 36-54 |
| Fludrocortisone | 0.1 | 12-36 |

A dose for one of the other anti-inflammatory steroid may be as set forth above for dexamethasone. Or it may by adjusted based on a selected dexamethasone does and using the above Conversion Table where the dose of the other anti-inflammatory steroid is calculated by [(approximate equivalent dose of the other anti-inflammatory steroid)/0.75]×(the selected dexamethasone dose). As shown in the Conversion Table, the half-life of the different steroids varies and this gives rise to a different duration of action. 8-12 hour half life is considered short-acting, 18-36 hour half-life is considered intermediate-acting, and 36-54 hour half-life is considered long-acting. One guideline to choosing one anti-inflammatory steroid, or a combination of two or more, may be the duration of action for each anti-inflammatory steroid. For example, a combination of more than one anti-inflammatory steroid administered may include two or three different durations of action. A dose of the at least one anti-inflammatory drug selected from anti-inflammatory steroids may comprise an amount of one or more of cortisone, hydrocortisone, methylprednisolone, prednisolone, prednisone, triamcinolone, betamethasone, fludrocortisone or dexamethasone to be an equivalent to a dose of dexamethasone as described herein.

For treatment of systemic inflammation in COVID-19 patients a dose of anti-inflammatory drug administered may be as described above. A dose may be 6 mg dexamethasone. When COVID-19 patients develop cytokine release syndrome (CRS), also known as cytokine storm, a dose of dexamethasone may be 20 mg, and the administration may be 1×-3×/day. In such patients, a combination of 6 mg dexamethasone and low dose RJX at 0.2-0.3 cc/kg was effective in reversing the systemic inflammation.

### Embodiments

The following list includes particular embodiments of the present invention. But the list is not limiting and does not exclude embodiments described elsewhere herein or alternate embodiments, as would be appreciated by one of ordinary skill in the art.
1. A pharmaceutical composition for intravenous or topical delivery to a mammal, the pharmaceutical composition comprising magnesium sulfate, ascorbic acid, thiamine, and niacinamide at a ratio (w/w) of 72 to 108 magnesium sulfate: 80 to 120 ascorbic acid: 5.6 to 8.4 thiamine: 10.4 to 15.6 niacinamide; or 81 to 99 magnesium sulfate: 90 to 110 ascorbic acid: 6.3 to 7.7 thiamine: 11.7 to 14.3 niacinamide; or 90 magnesium sulfate: 100 ascorbic acid: 7 thiamine: 13 niacinamide, preferably where the pharmaceutical composition further comprises one or more anti-inflammatory agent, preferably where the one or more anti-inflammatory agent comprises one or more anti-inflammatory drug selected from anti-inflammatory steroids, preferably where the one or more anti-inflammatory drug comprises dexamethasone, preferably where the concentration of dexamethasone is at a concentration such that one volume to be administered includes a dexamethasone dose of 0.75-40 mg, 1 mg-40, 1-2 mg, 4-8 mg, 10-20 mg, or the concentration of dexamethasone is such that one volume to be administered includes a dexamethasone dose of 0.75, 1, 2, 3, 4, 5, 6, 7, 8 9 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 mg, or a dose in a range between any two of the foregoing. Where an anti-inflammatory steroid(s) other than or in addition to dexamethasone is chosen, the dose(s) may be calculated based on the above Conversion Table and a desired dexamethasone selected from the foregoing dexamethasone doses.
2. The pharmaceutical composition of embodiment 1 further comprising pyridoxin and riboflavin, and the magnesium sulfate, ascorbic acid, thiamine, niacinamide, pyridoxin, and riboflavin are at a ratio (w/w) of 72 to 108 magnesium sulfate: 80 to 120 ascorbic acid: 5.6 to 8.4 thiamine: 10.4 to 15.6 niacinamide: 10.4 to 15.6 pyridoxin: 0.24 to 0.36 riboflavin; or 81 to 99 magnesium sulfate: 90 to 110 ascorbic acid: 6.3 to 7.7 thiamine: 11.7 to 14.3 niacinamide: 11.7 to 14.3 pyridoxin: 0.27 to 0.33 riboflavin; or 90 magnesium sulfate: 100 ascorbic acid: 7 thiamine: 13 niacinamide: 13 pyridoxin: 0.3 riboflavin.
3. The pharmaceutical composition of embodiment 1 or 2 further comprising a buffering agent.
4. The pharmaceutical composition of embodiment 3, wherein the buffering agent comprises sodium bicarbonate, lactate, acetate, gluconate or maleate.
5. The pharmaceutical composition of any one of embodiments 1-4 further comprising a diluent.
6. The pharmaceutical composition of embodiment 5, wherein the diluent comprises normal saline, water for injection, or a commonly used intravenous solution.
7. The pharmaceutical composition of any one of embodiments 1-6, wherein the magnesium sulfate is at a concentration of 0.7 to 0.9 mg/mL, the ascorbic acid is at a concentration of 0.8 to 1.0 mg/mL, the thiamine is at a concentration of 0.05 to 0.07 mg/mL, and the niacinamide is at a concentration of 0.105 to 0.150 mg/mL.
8. The pharmaceutical composition of any of embodiments 2-7, wherein the pyridoxin is at a concentration of 0.105 to 0.150 mg/mL and the riboflavin is at a concentration of 0.002 to 0.003 mg/mL.
9. The pharmaceutical composition of any one of embodiments 1-8 further comprising cyanocobalamin.
10. The pharmaceutical composition of any of embodiments 1-9 further comprising at least one of an antioxidant or the one or more anti-inflammatory agent, which may include an anti-inflammatory drug.
11. The pharmaceutical composition of embodiment 10, wherein the at least one of an antioxidant or an anti-inflammatory agent are selected from Cox-2 or Cox1 inhibitors, steroids, zinc, copper, selenium, Vitamin E, and Vitamin A.
12. The pharmaceutical composition of any of embodiments 1-11 further comprising the one or more anti-inflammatory drug selected from anti-inflammatory steroids.
13. The pharmaceutical composition of embodiment 12, wherein the anti-inflammatory steroids comprise at least one of cortisone, hydrocortisone, methylprednisolone, prednisolone, prednisone, triamcinolone, betamethasone, fludrocortisone, or dexamethasone, or a pharmaceutically acceptable salt thereof.
14. The pharmaceutical composition of embodiment 12, wherein the one or more anti-inflammatory drug comprises dexamethasone.
15. The pharmaceutical composition of embodiment 14, wherein the concentration of dexamethasone is the concentration such that one volume to be administered includes a dexamethasone dose of 0.75-40 mg, 1 mg-40. 1-2 mg, 4-8 mg, 10-20 mg; or the concentration of dexamethasone is such that one volume to be administered includes a dexamethasone dose of 0.75, 1, 2, 3, 4, 5, 6, 7, 8 9 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 mg, or a dose in a range between any two of the foregoing.
16. A pre-formulation comprising a concentrate of the pharmaceutical composition of any one of embodiments 1-15, wherein when present:
the magnesium sulfate is at a concentration of 50 to 100×(0.7 to 0.9 mg/mL), the ascorbic acid is at a concentration of 50 to 100×(0.8 to 1.0 mg/mL);
the thiamine is at a concentration of 50 to 100×(0.05 to 0.07 mg/mL);
the niacinamide is at a concentration of 50 to 100×(0.105 to 0.150 mg/mL);
the pyridoxin is at a concentration of 50 to 100×(0.105 to 0.150 mg/mL);
the riboflavin is at a concentration of 50 to 100×(0.002 to 0.003 mg/mL);
the cyanocobalamin is at a concentration of 50 to 100×(0.0015 to 0.0030 mg/mL); and
the anti-inflammatory steroid is at a concentration of 50 to 100× the value given in embodiment 1 or embodiment 15.
17. A method of treating an inflammatory condition in a mammal comprising administering to the mammal an effective amount of the pharmaceutical composition of any one of embodiments 1-15; or administering to the mammal an effective amount of the composition of any one of embodiments 1-15 minus the at least one anti-inflammatory drug and separately co-administering the at least one anti-inflammatory drug; preferably where the at least one anti-inflammatory drug comprises dexamethasone, preferably where the concentration of dexamethasone is at a dose of 0.75-40 mg, 1 mg-40. 1-2 mg, 4-8 mg, 10-20 mg; or a dose of 0.75, 1, 2, 3, 4, 5, 6, 7, 8 9 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 mg, or a dose in a range between any two of the foregoing. Where an anti-inflammatory steroid(s) other than or in addition to dexamethasone is chosen, the dose(s) may be calculated based on the above Conversion Table and a desired dexamethasone dose selected from the foregoing dexamethasone doses.
18. The method of embodiment 17, wherein the mammal is a human.
19. The method of embodiment 17, wherein the mammal is a dog, cat, or horse.
20. The method of any one of embodiments 17-19, wherein the administering is intravenous infusion, subcutaneous injection, intraarterial injection, via inhalation, or via nasal spray.
21. The method of any one of embodiments 17-19, wherein the administering comprises daily intravenous infusions.
22. The method of embodiment 21, wherein the daily intravenous infusions are for 1-12 cycles of 7-28 consecutive days, wherein 0-365 days separates each cycle.
23. The method of embodiment 21 or 22, wherein the daily intravenous infusion has a dose of 0.025 mL/kg to 2.5 mL/kg of the pharmaceutical composition administered over 15-60 minutes.
24. The method of any of embodiments 17-23, wherein the administering comprises a dose of 2.5 mL/kg of the pharmaceutical composition.
25. The method of any of embodiments 17-23, wherein the administering comprises a dose of 100 ml.
26. The method of any one of embodiments 17-25, wherein the inflammatory condition is one affecting the joints, skin, skeletal muscle, blood vessels, liver, gall bladder, lungs, heart, brain, meninges, gastrointestinal system, urinary bladder, urethra, or kidneys, or a systemic inflammation.
27. The method of embodiment 26, wherein the inflammatory condition affecting the joints is arthritis.
28. The method of embodiment 26, wherein the inflammatory condition affecting the skin is dermatitis.
29. The method of embodiment 26, wherein the inflammatory condition affecting the skeletal muscle is myositis.
30. The method of embodiment 26, wherein the inflammatory condition affecting the blood vessels is vasculitis, vascular leak syndrome, capillary leak syndrome, or retinitis.
31. The method of embodiment 26, wherein the inflammatory condition affecting the liver is hepatitis.
32. The method of embodiment 26, wherein the inflammatory condition affecting the gall bladder is cholecystitis.
33. The method of embodiment 26, wherein the inflammatory condition affecting the lungs is pneumonitis.
34. The method of embodiment 26, wherein the inflammatory condition affecting the heart is myocarditis, pericarditis, or endocarditis.
35. The method of embodiment 26, wherein the inflammatory condition affecting the brain is encephalitis.
36. The method of embodiment 26, wherein the inflammatory condition affecting the meninges is meningitis.
37. The method of embodiment 26, wherein the inflammatory condition affecting the gastrointestinal system is gastritis, colitis, enteritis, or esophagitis.
38. The method of embodiment 26, wherein the inflammatory condition affecting the urinary bladder is cystitis.
39. The method of embodiment 26, wherein the inflammatory condition affecting the urethra is urethritis.
40. The method of embodiment 26, wherein the inflammatory condition affecting the kidneys is nephritis.
41. The method of any one of embodiments 17-25, wherein the inflammatory condition a systemic inflammation.
42. The method of embodiment 41, wherein the systemic inflammation is sepsis, cytokine release syndrome, cytokine storm, graft versus host disease, or multi-organ auto-immune disease.
43. The method of embodiment 42, wherein the multi-organ auto-immune disease is Lupus/SLE.
44. The method of any one of embodiments 17-25, wherein the inflammatory condition is one caused by a toxic agent, radiation, an infection, obesity-related complications, autoimmune disease, bone marrow transplantation, organ transplantation, treatment with monoclonal antibodies, treatment with antibody-drug conjugates, treatment with bidirectional T-cell engagers, treatment with biologic(s), cancer, or cancer therapy.
45. The method of embodiment 44, wherein the toxic agent include alcohol, a chemotherapy drug, a poison, a controlled drug substance, or a chemical or biological warfare agent.
46. The method of embodiment 44, wherein the radiation is sunburn/UV radiation, or ionizing radiation from an irradiator or a radioisotope.
47. The method of embodiment 44, wherein the infection includes SARS-CoV-2 infection (COVID-19), viral infection, bacterial infection, or fungal infection.
48. The method of embodiment 44, wherein the obesity-related complications include metabolic syndrome.
49. The method of embodiment 44, wherein the biologic(s) are selected from recombinant therapeutic proteins, vaccines, and vaccine-like products.
50. The method of any of embodiments 17-25, wherein the mammal is a human with Ulcerative colitis, Crohn disease, Rheumatoid arthritis, hemophagocytic lymphohistiocytosis, chronic inflammatory demyelinating polyneuropathy, multiple sclerosis, sarcoidosis, rheumatic fever, Behcet disease, Mediterranean fever, inflammatory pelvic disease, interstitial cystitis, or Heliobacter pylori.
51. The method of any of embodiments 17-25, wherein the mammal is a human and the inflammatory condition is caused by infection of the human by the SARS-CoV-2 virus, COVID-19 in the human, or presence of SARS-CoV-2 virus spike protein in the human.
52. The method of any one of embodiments 17-51, wherein the pharmaceutical composition is administered to the mammal at 2.5 mL/kg or a fixed dose of 100 mL.
53. A method of treating a mammal in need thereof, the method comprising administering the pharmaceutical composition of any one of embodiments 1-15, or the composition of any one of embodiments 1-15 minus the at least one anti-inflammatory drug selected from anti-inflammatory steroids and co-administering the at least one anti-inflammatory drug, and wherein the mammal has (1) viral sepsis, cytokine storm, cytokine release syndrome, pneumonia, Kawasaki disease or ARDS caused by COVID-19, (2) bacterial sepsis, (3) fungal sepsis, (4) acute graft versus host disease, (5) fulminant hepatitis, (6) radiation pneumonitis, (7) acute flare of an inflammatory bowel disease such as ulcerative colitis or Crohn disease, or (8) a multi-system inflammation of any cause, optionally where the mammal is a patient affected by an inflammatory condition; preferably where the at least one anti-inflammatory drug comprises dexamethasone, preferably where the dose of dexamethasone is 0.75-40 mg, 1 mg-40. 1-2 mg, 4-8 mg, 10-20 mg; or a dose of 0.75, 1, 2, 3, 4, 5, 6, 7, 8 9 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 mg, or a dose in a range between any two of the foregoing. Where an anti-inflammatory steroid(s) other than or in addition to dexamethasone is chosen, the dose(s) may be calculated based on the above Conversion Table and a desired dexamethasone selected from the foregoing dexamethasone doses.
54. The method of embodiment 53, wherein the pharmaceutical composition is administered to the mammal at 2.5 mL/kg or a fixed dose of 100 mL.
55. A method of blocking the production and or release of the inflammatory cytokines in a mammal, the method comprising administering the pharmaceutical composition of any one of embodiments 1-15, or the composition of any one of embodiments 1-15 minus the at least one anti-inflammatory drug selected from anti-inflammatory steroids and co-administering the at least one anti-inflammatory drug, to the mammal, optionally wherein the mammal is a human, optionally the mammal is a patient affected by an inflammatory condition; preferably where the at least one anti-inflammatory drug comprises dexamethasone, preferably where the dose of dexamethasone is 0.75-40 mg, 1 mg-40. 1-2 mg, 4-8 mg, 10-20 mg; or a dose of 0.75, 1, 2, 3, 4, 5, 6, 7, 8 9 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 mg, or a dose in a range between any two of the foregoing. Where an anti-inflammatory steroid(s) other than or in addition to dexamethasone is chosen, the dose(s) may be calculated based on the above Conversion Table and a desired dexamethasone selected from the foregoing dexamethasone doses.
56. The method of embodiment 55 wherein the inflammatory cytokine is Tumor necrosis factor alpha (TNF-α), interleukin-6 (IL-6), or transforming growth factor beta (TGF-3).
57. A method of preventing or treating a human or animal disease by inhibiting production or release of an inflammatory Cytokine, optionally by administering the pharmaceutical composition of any one of embodiments 1-15, or the composition of any one of embodiments 1-15 minus the at least one anti-inflammatory drug selected from anti-inflammatory steroids and co-administering the at least one anti-inflammatory drug, to the human or animal, optionally wherein the human or animal is a human person, optionally wherein the human person is affected by an inflammatory condition. Preferably the at least one anti-inflammatory drug comprises dexamethasone, preferably where the dose of dexamethasone is 0.75-40 mg, 1 mg-40. 1-2 mg, 4-8 mg, 10-20 mg; or a dose of 0.75, 1, 2, 3, 4, 5, 6, 7, 8 9 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 mg, or a dose in a range between any two of the foregoing. Where an anti-inflammatory steroid(s) other than or in addition to dexamethasone is chosen, the dose(s) may be calculated based on the above Conversion Table and a desired dexamethasone selected from the foregoing dexamethasone doses.
58. A method of treating or reducing oxidative stress in cells and/or tissue by administering the pharmaceutical composition of any one of embodiments 1-15, or the composition of any one of embodiments 1-15 minus the at least one anti-inflammatory drug selected from anti-inflammatory steroids and co-administering the at least one anti-inflammatory drug, to a mammal, optionally wherein the mammal is a human, optionally wherein the human is a patient affected by an inflammatory condition, optionally wherein the patient affected by an inflammatory condition has cells and/or tissue affected by oxidative stress; preferably where the at least one anti-inflammatory drug comprises dexamethasone, preferably where the dexamethasone is at a dose of dexamethasone is 0.75-40 mg, 1 mg-40. 1-2 mg, 4-8 mg, 10-20 mg; or a dose of 0.75, 1, 2, 3, 4, 5, 6, 7, 8 910, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 mg, or a dose in a range between any two of the foregoing. Where an anti-inflammatory steroid(s) other than or in addition to dexamethasone is chosen, the dose(s) may be calculated based on the above Conversion Table and a desired dexamethasone selected from the foregoing dexamethasone doses.
59. The method of embodiment 58, wherein the oxidative stress is treated or reduced by preventing peroxidation of membranes.
60. The method of embodiment 58, wherein the oxidative stress is treated or reduced by increasing the levels of anti-oxidant enzymes, optionally where the anti-oxidant enzymes are one or more selected from catalase, superoxide dismutase and glutathione peroxidase.
61. The method of embodiment 58, wherein the oxidative stress is treated or reduced by increasing the blood and tissue levels of ascorbic acid and niacinamide and thiamine.
62. A method of treating a COVID-19 patient comprising administering an effective amount of the pharmaceutical composition of any one of embodiments 1-15, or the composition of any one of embodiments 1-15 minus the at least one anti-inflammatory drug selected from anti-inflammatory steroids and co-administering the at least one anti-inflammatory drug, to the patient; preferably where the at least one anti-inflammatory drug comprises dexamethasone, preferably where the dexamethasone is at a dose of 0.75-40 mg, 1 mg-40. 1-2 mg, 4-8 mg, 10-20 mg; or a dose of 0.75, 1, 2, 3, 4, 5, 6, 7, 8 9 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 mg, or a dose in a range between any two of the foregoing. Where an anti-inflammatory steroid(s) other than or in addition to dexamethasone is chosen, the dose(s) may be calculated based on the above Conversion Table and a desired dexamethasone selected from the foregoing dexamethasone doses.
63. The method of embodiment 62, wherein the administering is intravenous infusion, subcutaneous injection, intraarterial injection, via inhalation, or via nasal spray.
64. The method of embodiment 62 or 63, wherein the administering comprises daily intravenous infusions.
65. The method of embodiment 64, wherein the daily intravenous infusions are for 1-12 cycles of 7-28 consecutive days, wherein 0-365 days separates each cycle.
66. The method of embodiment 64 or 65, wherein the daily intravenous infusion has a dose of 0.025 mL/kg to 2.5 mL/kg of the pharmaceutical composition administered over 15-60 minutes.
67. The method of any of embodiments 62-66, wherein the administering comprises a dose of 2.5 mL/kg of the pharmaceutical composition.
68. The method of any of embodiments 62-66, wherein the administering comprises a dose of 100 ml.

Further embodiments herein may be formed by supplementing an embodiment with one or more element from any one or more other embodiment herein, and/or substituting one or more element from one embodiment with one or more element from one or more other embodiment herein.

Examples—The following non-limiting examples are provided to illustrate particular embodiments. The embodiments throughout may be supplemented with one or more detail from one or more example below, and/or one or more element from an embodiment may be substituted with one or more detail from one or more example below.

New treatment platforms that can prevent acute respiratory distress syndrome (ARDS) or reduce its mortality rate in high-risk COVID-19 patients, such as those with an underlying cancer, are urgently needed. Rejuveinix (RJX) is a formulation of several vitamins, including ascorbic acid (Vitamin C), cyanocobalamin (Vitamin B12), thiamine hydrochloride (Vitamin B1), riboflavin 5′ phosphate (Vitamin B2), niacinamide (Vitamin B3), pyridoxine hydrochloride (Vitamin B6), calcium D-pantothenate, and magnesium sulphate as a potent calcium antagonist, representing components that have been studied in animal models of septic shock and ARDS as well as clinical studies in septic patients. RJX has a very favorable safety profile in human subjects. RJX is being developed as an anti-inflammatory and anti-oxidant treatment platform for patients with sepsis, including COVID-19 patients with viral sepsis and ARDS. A nonclinical pharmacodynamic study it analyzed if RJX can improve the survival outcome of mice challenged with an otherwise invariably fatal dose of LPS-GalN in a model of sepsis, ARDS and multi-organ failure. RJX exhibited potent protective anti-CRS and anti-ARDS activity in the LPS-GalN model at clinically safe low dose levels.

The ability of RJX to prevent fatal shock, ARDS, and multi-organ failure was examined in the well-established lipopolysaccharide (LPS)-Galactosamine (GalN) mouse model of sepsis and ARDS. Standard methods were employed for the statistical analysis of data in both studies. No participant developed serious adverse events (SAEs) or Grade 3-Grade 4 adverse events (AEs) or prematurely discontinued participation from the study. In a non-clinical study, RJX exhibited potent and dose-dependent protective activity, decreased the inflammatory cytokine responses (IL-6, TNF-α, TGF-3), and improved survival in the LPS-GalN mouse model of ARDS. Histopathological examinations showed that RJX attenuated the LPS-GalN induced acute lung injury (ALI) and pulmonary edema as well as liver damage. Conclusion. RJX showed a very favorable safety profile and tolerability in human subjects. It show potential to favorably affect the clinical course of high-risk COVID-19 by preventing ARDS and its complications.

### Example 1.1.1.—RJX Prevents Proinflammatory Cytokine Responses and Improves Survival Outcome after LPS-GalN Induced Sepsi

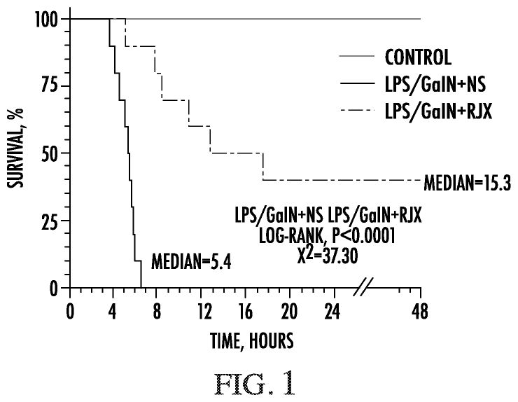

One hundred (100) percent (%) of untreated control mice remained alive throughout the experiment. By comparison, 100% of LPS-GalN injected mice died at a median of 5.4 hours (FIG. 1). RJX was examined for its protective activity at a dose level, which is >10-fold lower than its maximum tolerated dose (MTD) of 0.759 mL/kg for human subjects (viz.; 4.2 mL/kg of a 6-fold diluted solution) and RJX-treated mice had an improved survival outcome after being injected with LPS-GalN. In contrast to the invariably fatal treatment outcome of vehicle-treated control mice, 40% of mice treated with RJX (N=10) remained alive with a 2.8-fold longer median time survival time of 15.3 hours (Log-rank P=0.004; Z-score: −4.059, P<0.001). See FIG. 1.

Groups of 10 BALB/C mice were treated with i.p injections of RJX (4.2 mL/kg, 0.5 ml/mouse) or vehicle 2 hours before or post-injection of LPS-GalN. Except for untreated mice (Control), each mouse received 0.5 ml of LPS-GalN (consisting of 100 ng of LPS plus 8 mg of D-galactosamine) i.p. Survival is shown as a function of time after the LPS-GalN challenge. Depicted are the survival curves of the different treatment groups. Depicted are the Kaplan-Meier survival curves for each group along with the median survival times and log-rank P-value for the comparison of LPS-GalN+RJX group with the LPS/GalN+NS group.

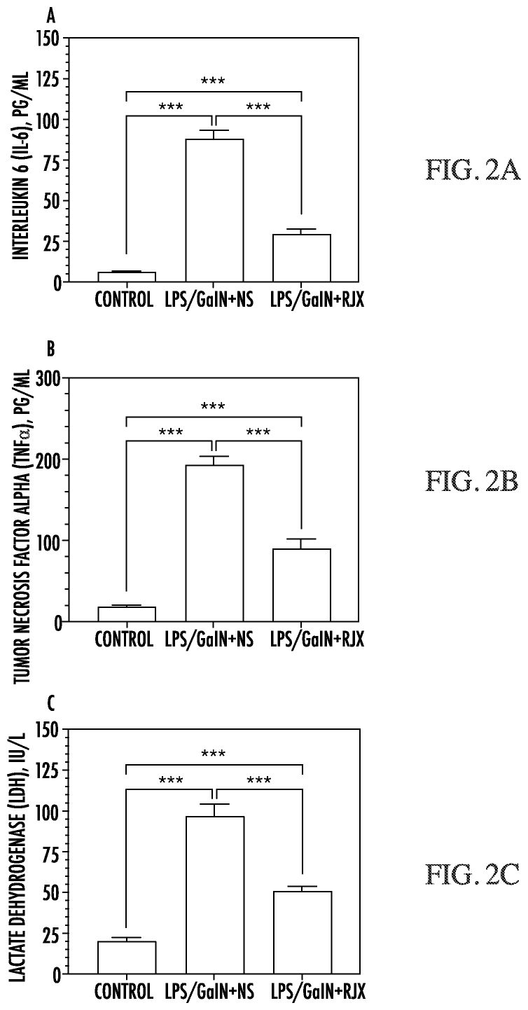

In LPS-GalN challenged control mice not receiving any RJX treatments, serum IL-6, TNF-α and LDH levels were drastically increased at the time of death which is consistent with a “cytokine storm” and marked systemic inflammation. See FIGS. 2A, 2B, and 2C. Each bar represents mean and standard deviation. Groups of 10 BALB/C mice were treated with i.p injections of RJX (4.2 mL/kg, 0.5 ml/mouse) or vehicle 2 hours before or post-injection of LPS-GalN. Except for untreated mice (Control), each mouse received 0.5 ml of LPS-GalN (consisting of 100 ng of LPS plus 8 mg of D-galactosamine) i.p. (ANOVA and Tukey's post-hoc test. Statistical significance between groups is shown by: *** P<0.001).

In contrast, RJX-treated mice that died after LPS-GalN injection had significantly lower levels of IL-6, TNF-α, as well as LDH (FIG. 2C), and died much later than the vehicle-treated mice (FIG. 1). These results of mice that died within 24 hours after the LPS-GalN challenge demonstrate that RJX decreased the proinflammatory cytokine responses of mice injected with LPS-GalN, and improved the survival time of mice.

### Example 1.1.2.—RJX Reduces the Oxidative Stress in the Lungs and Attenuates ALI after LPS-GalN Induced Sepsis, Cytokine Storm, and Systemic Inflammation

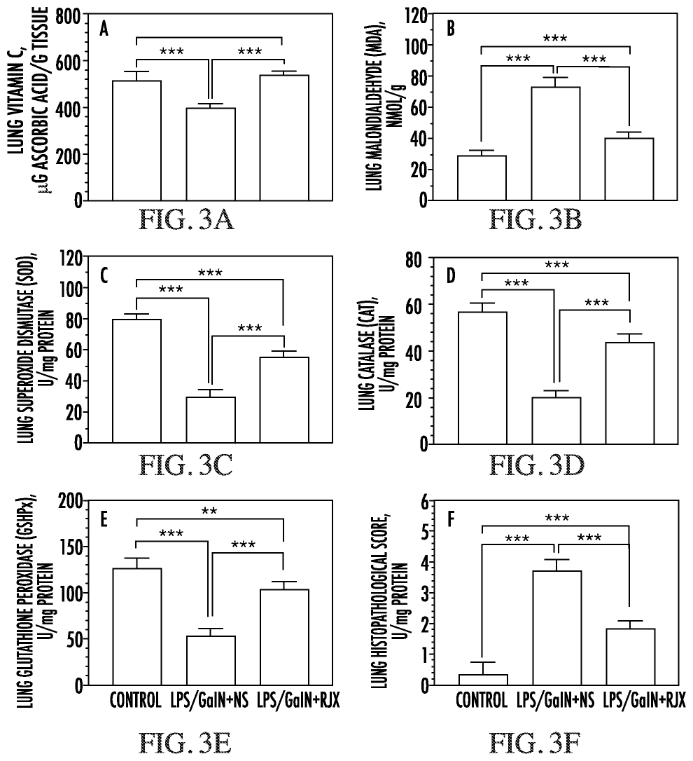

When compared to untreated control mice, the lung MDA levels measuring lipid peroxidation were markedly elevated in LPS-GalN challenged mice (6.5±0.5 vs. 2.6±0.4 nmol/g, P<0.0001). Conversely, their tissue levels of the antioxidant enzymes SOD (30.5±1.2 U/mg vs. 80.4±1.6 U/mg, P<0.0001), CAT (19.9±1.1 U/mg vs. 56.7±1.4 U/mg, P<0.0001), GSH-Px (54.2±3.1 U/mg vs. 126.4±4.1 U/mg, P<0.0001), as well as ascorbic acid (54.5±0.1 μg/g vs. 398.2±0.1 μg/g, P<0.0001) in the lung were markedly reduced consistent with severe oxidative stress in the lung tissue. See FIGS. 3A, 3B, 3C, 3D, 3E, and 3F. Groups of 10 BALB/C mice were treated with i.p injections of RJX (4.2 mL/kg, 0.5 ml/mouse) or vehicle 2 hours before or post-injection of LPS-GalN. Except for untreated mice (Control), each mouse received 0.5 ml of LPS-GalN (consisting of 100 ng of LPS plus 8 mg of D-galactosamine) i.p. After LPS/GalN challenge for 24 h, the lungs of the mice were harvested. The lung score was graded according to a 5-point scale from 0 to 4 as follows: 0, I, 2, 3 and 4 represented no damage, mild damage, moderate damage, severe damage and very severe damage, respectively. (Kruskal-Wallis test and Mann Whitney test. Statistical significance between groups is shown by: *** P<0.001).

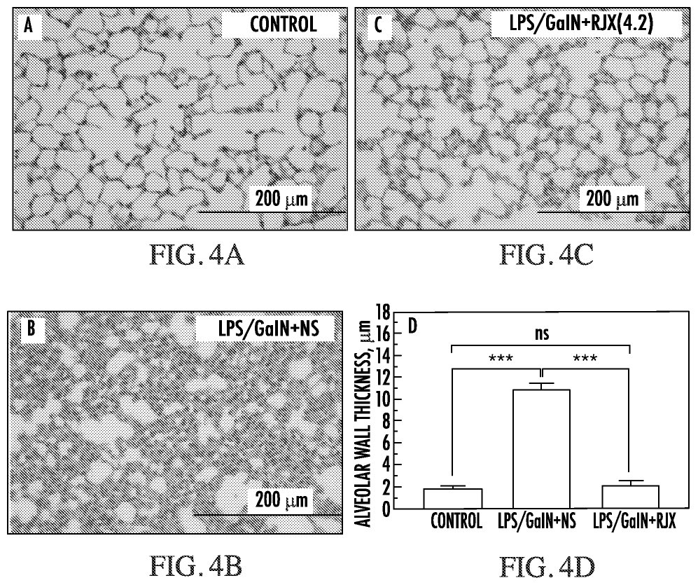

Histopathological examination of hematoxylin-eosin (H/E)-stained lung tissues from LPS-GalN injected mice showed histological changes consistent with severe acute ALI, including alveolar hemorrhage, thickening of alveolar wall, edema/congestion, and leukocyte infiltration (FIGS. 3A-3F). These changes were not observed in the lung tissues of mice in the control group that was not injected with LPS-GalN. RJX decreased the lung MDA levels, and normalized the reduced levels of the antioxidant enzymes SOD, CAT, GSH-Px and ascorbic acid (FIGS. 3A-3F). Notably, RJX attenuated the LPS-GalN induced ALI as evidenced by significantly less damage in the lungs of RJX-treated mice. The ALI scores depicted in FIGS. 3A-3F show a dose-dependent protective effect of RJX with a highly statistically significant reduction of the ALI score for the lungs of RJX-treated mice. The RJX prevented the development of pulmonary edema in LPS-GalN challenged mice, as documented by the decrease of the alveolar wall thickness was substantially decreased to near normal values in RJX-treated mice (FIGS. 3A-3F, FIGS. 4A-4D).

Referring to FIG. 4A-4D, mice were treated with i.p injections of 6-fold diluted RJX (4.2 mL/kg, 0.5 ml/mouse) or NS 2 hours before and 2 hours post-injection of LPS-GalN. Except for untreated mice (Control), each mouse received 0.5 ml of LPS-GalN (consisting of 100 ng of LPS plus 8 mg of D-galactosamine) i.p. The alveolar wall thickness was markedly increased in mice treated with NS consistent with massive pulmonary edema. RJX treatments were associated with prevention of pulmonary edema as evidenced by near normal alveaolar wall thickness measurements. H&E ×400.

### Example 1.1.3.—RJX Reduces the Oxidative Stress in the Liver and Attenuates Acute Liver Damage after LPS-GalN Induced Sepsis and Systemic Inflammation

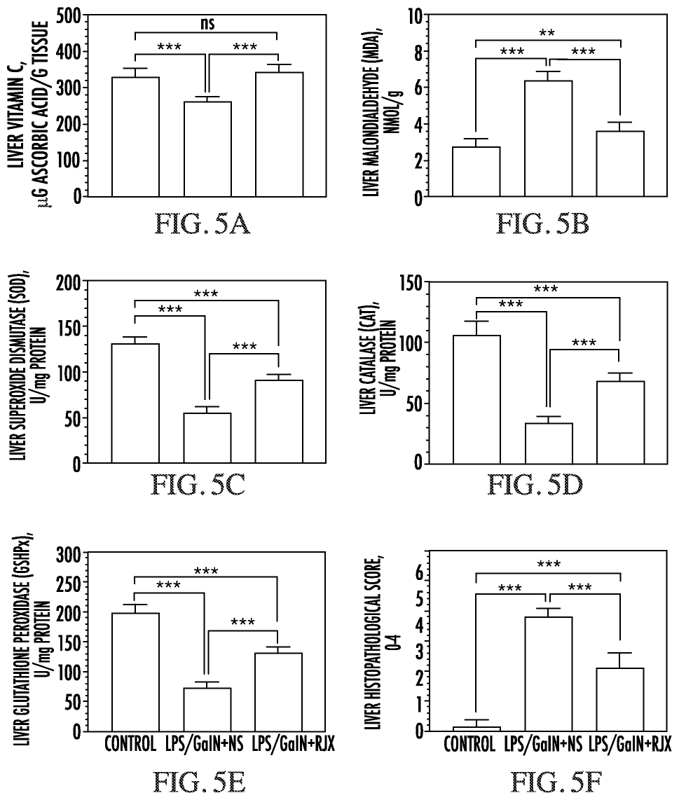

The liver MDA levels measuring lipid peroxidation were markedly elevated and the levels of the antioxidant enzymes SOD, CAT, GSH-Px, as well as ascorbic acid were markedly reduced in LPS-GalN treated mice consistent with severe oxidative stress (FIGS. 5A-5F).

Furthermore, RJX decreased the liver MDA levels and normalized in a dose-dependent manner the reduced levels of the antioxidant enzymes SOD, CAT, and GSH-Px as well as ascorbic acid.

In FIGS. 5A-5F, each bar represents mean and standard deviation. Groups of 10 BALB/C mice were treated with i.p injections of RJX (4.2 mL/kg, 0.5 ml/mouse) or vehicle 2 hours before or post-injection of LPS-GalN. Except for untreated mice (Control), each mouse received 0.5 ml of LPS-GalN (consisting of 100 ng of LPS plus 8 mg of D-galactosamine) i.p. (ANOVA and Tukey's post-hoc test. Statistical significance between groups is shown by: ** P<0.01; *** P<0.001).

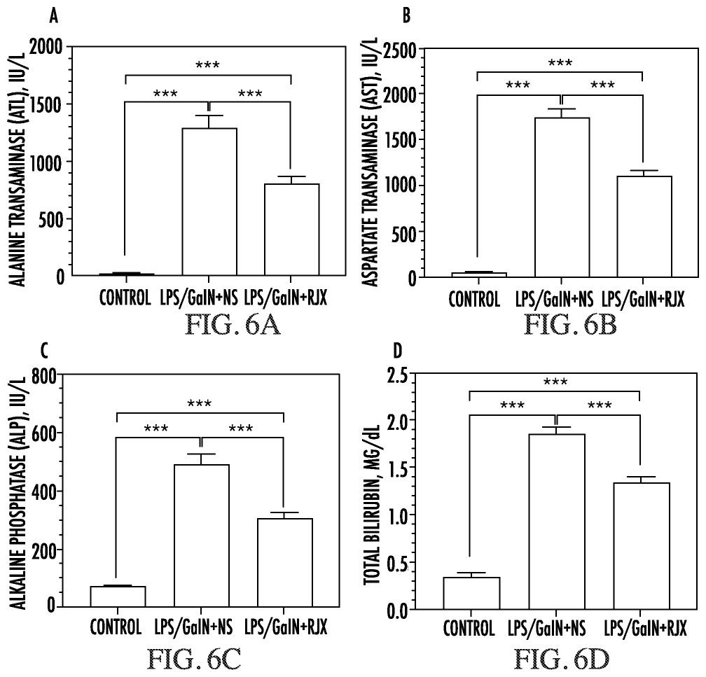

FIGS. 1A-6D show the Effect Of Rejuveinix (RJX) On Alanine Transaminase (ALT; FIG. 6A), Aspartate Transaminase (AST; FIG. 6B), Alkaline Phosphatase (ALP; FIG. 6C), And Total Bilirubin (FIG. 6D) In Lipopolysaccharide-Galactosamine (LPS-GalN) Challenged Mice. In FIGS. 6A-6D, Each bar represents mean and standard deviation. Groups of 10 BALB/C mice were treated with i.p injections of RJX (4.2 mL/kg, 0.5 ml/mouse) or vehicle 2 hours before or post-injection of LPS-GalN. Except for untreated mice (Control), each mouse received 0.5 ml of LPS-GalN (consisting of 100 ng of LPS plus 8 mg of D-galactosamine) i.p. (ANOVA and Tukey's post-hoc test. Statistical significance between groups is shown by: *** P<0.001).

LPS-GalN caused significant liver damage and hepatic dysfunction in mice with a marked elevation of the liver enzymes as well as total bilirubin when compared to the enzyme levels in untreated healthy control mice: ALT (1297.1±106.8 vs. 28.3±4.5; 46-fold elevation; P<0.0001), AST (1756.0±96.8 vs. 57.9±10.3; 30-fold elevation; P<0.0001), ALP (491.2±33.9 vs. 69.9±6.7; 7-fold elevation; P<0.0001), total bilirubin (TBIL) (1.9±0.1vs. 0.3±0.1; 6-3 fold elevation; P<0001) (FIG. 1). RJX-treated mice that died following the LPS-GalN challenge had significantly lower, albeit still abnormal, levels for liver enzymes and TBIL (FIGS. 6A-6D).

Histopathologic examination of the livers from LPS-GalN treated mice showed massive generalized hepatocyte vacuolation and necrosis corresponding to >25% necrosis and destruction of the liver parenchyma, severe lobular inflammation involving >50% of the liver parenchyma, and severe portal inflammation involving more than >50% of portal tracts. RJX suppressed the LPS-GalN induced liver injury and inflammation, as documented by reduction of the histopathological scores measuring liver damage (FIG. 5F).

### Example 1.1.4.—RJX Reduces the Oxidative Stress in the Heart and Attenuates Acute Myocardial Injury after LPS-GalN Induced Sepsis, Systemic Inflammation, Shock, ARDS and Multi-Organ Failure

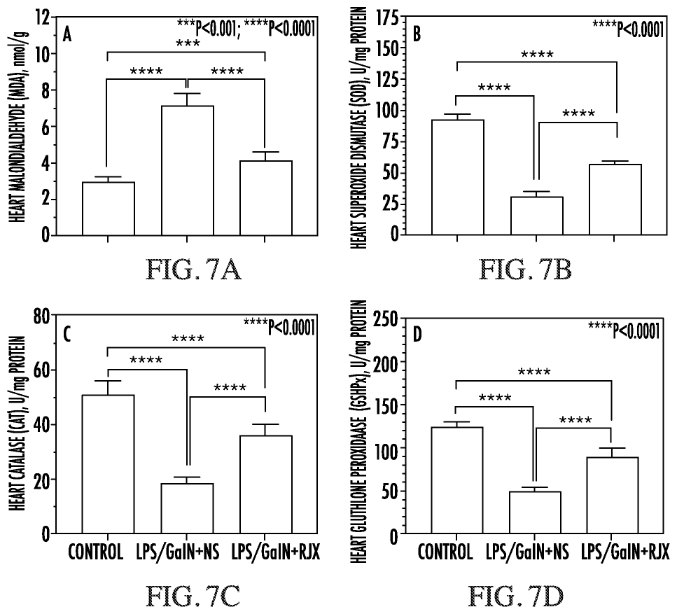

FIGS. 7A-7D illustrate Heart Tissue-Level In Vivo Anti-Oxidant Activity of Rejuveinix (RJX) in the LPS-GalN Mouse Model of Sepsis, Systemic Inflammation, shock, ARDS and Multi-organ Failure. Mice were treated with i.p injections of RJX (4.2 mL/kg, 0.5 ml/mouse) or NS 2 hours before and 2 hours post-injection of LPS-GalN. Except for untreated mice (Control), each mouse received 0.5 ml of LPS-GalN (consisting of 100 ng of LPS plus 8 mg of D-galactosamine) i.p. The depicted bars represent the mean and standard deviation for the indicated parameters. ANOVA and Tukey's post-hoc test were used for comparing the results among different treatment groups. Statistical significance between groups is shown by: *** P<0.001; **** P<0.0001).

No histopathologic lesions were observed in the heart of any of the mice challenged with LPS-GalN. However, the serum cardiac troponin I (cTnl) levels were markedly elevated at the time of death after the LPS-GalN challenge, which is consistent with myocardial injury. Furthermore, the cardiac MDA levels measuring lipid peroxidation were markedly elevated and the levels of the antioxidant enzymes SOD, CAT, and GSH-Px were markedly reduced in the heart of the LPS-GalN treated mice consistent with severe oxidative stress (FIGS. 7A-7D).

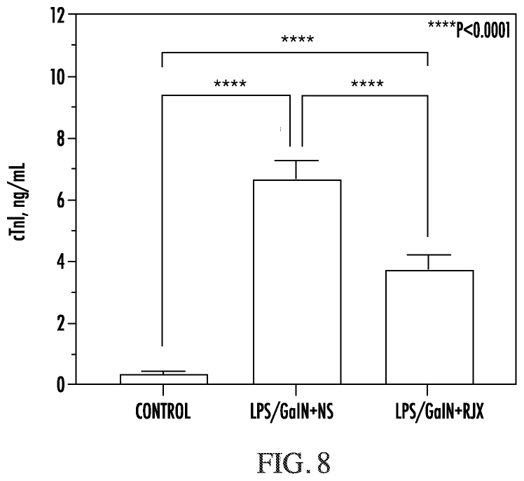

Referring to FIG. 8, RJX attenuated the myocardial injury as documented by a significant reduction of the serum cTnl levels. It also decreased the elevated MDA levels and improved the reduced levels of the antioxidant enzymes SOD, CAT, and GSH-Px, consistent with a significant reduction of oxidative stress.

FIG. 8 illustrates Effect Of Rejuveinix (RJX) On Serum cTni Level In LPS-GalN Mouse Model Of Sepsis, Systemic Inflammation, Shock, And Multi-Organ Failure. Mice were treated with i.p injections of RJX (4.2 mL/kg, 0.5 ml/mouse) or NS 2 hours before and 2 hours post-injection of LPS-GalN. Except for untreated mice (Control), each mouse received 0.5 ml of LPS-GalN (consisting of 100 ng of LPS plus 8 mg of D-galactosamine) i.p. The depicted bars represent the mean and standard deviation for the indicated parameters. ANOVA and Tukey's post-hoc test were used for comparing the results among different treatment groups. Statistical significance between groups is shown by: *** P<0.001; **** P<0.0001).

### Example 1.1.5.—RJX Reduces the Oxidative Stress in the Brain after LPS-GalN Challenge in the LPS-GalN Model of Sepsis, Systemic Inflammation, Shock, and Multiorgan Failure

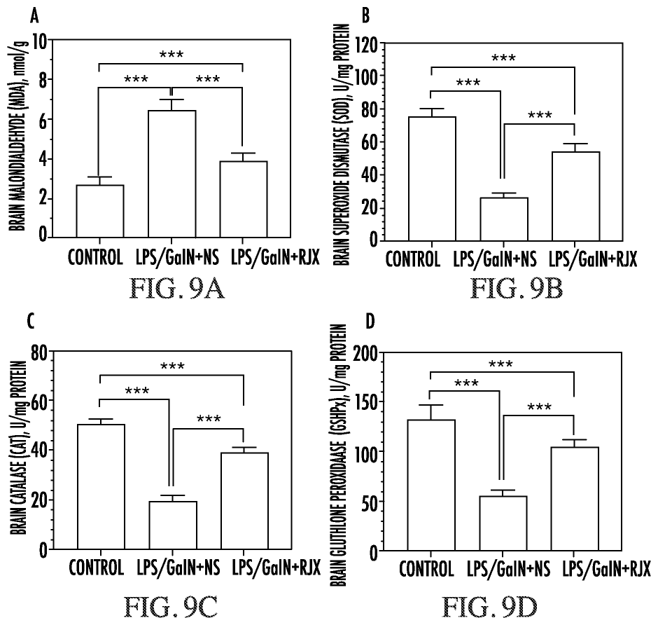

Referring to FIGS. 9A-9D, no histopathologic brain lesions were observed in any of the mice challenged with LPS-GalN. However, the liver MDA levels measuring lipid peroxidation were markedly elevated and the levels of the antioxidant enzymes SOD, CAT, and GSH-Px were markedly reduced in LPS-GalN treated mice consistent with severe oxidative stress. Furthermore, RJX decreased the liver MDA levels and normalized in a dose-dependent manner the reduced levels of the antioxidant enzymes SOD, CAT, and GSH-Px.

FIGS. 9A-9D illustrate Effect Of Rejuveinix (RJX) On Brain Malondialdehyde (MDA; FIG. 9A), Superoxide Dismutase (SOD; FIG. 9B), Catalase (CAT; FIG. 9C), And; Glutathione Peroxidase (GSHPx; FIG. 9D) In Lipopolysaccharide-Galactosamine (LPS-GalN) Challenged Mice. Each bar represents mean and standard deviation. Groups of 10 BALB/C mice were treated with i.p injections of RJX (4.2 mL/kg, 0.5 ml/mouse) or vehicle 2 hours before or post-injection of LPS-GalN. Except for untreated mice (Control), each mouse received 0.5 ml of LPS-GalN (consisting of 100 ng of LPS plus 8 mg of D-galactosamine) i.p. (ANOVA and Tukey's post-hoc test. Statistical significance between groups is shown by: *** P<0.001).

Notably, our results in the aforementioned animal model have demonstrated that RJX treatments result in a marked reduction of IL-6 and TNF-α in the serum, and IL-6, TNF-α as well as TGF-β levels in the lungs as well as liver of mice challenged with LPS-GalN in a dose-dependent fashion. It is postulated that the RJX-mediated suppression of inflammatory cytokine expression in the lungs and other tissues will accelerate the resolution of systemic inflammation of MIS-C patients by reducing the contributions of these cytokines to MIS-C and its complications. Therefore, RJX has the potential to emerge as a clinically useful adjunct to the best available standard of care and supportive care in pediatric COVID-19 patients who develop MIS-C.

### Example 1.1.6.—In Vivo Protective Activity of Delayed-Onset RJX Treatments in the LPS-GalN Model of Sepsis, Systemic Inflammation, Shock, and Multiorgan Failure

It was next sought to determine whether RJX could also improve the survival outcome of LPS-GalN challenged mice if the treatment is delayed until after the onset of the inflammatory cytokine response.

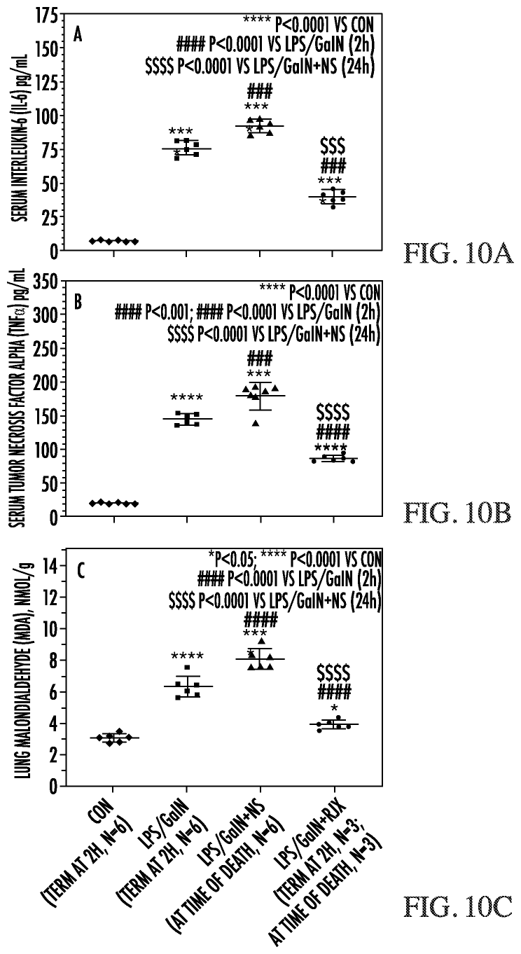

FIGS. 10A, 10B, and 10C illustrate Effect Of Rejuveinix (RJX) On Serum Interleukin-6 (IL-6; FIG. 10A), Tumor Necrosis Factor Alpha (TNF-α; FIG. 10B) And Lung Malondialdehyde (MDA; FIG. 10C) Mice Challenged With LPS-GalN. Mice were treated with i.p injections of RJX (4.2 mL/kg, 0.5 ml/mouse) or NS 2 hours and 3 hours post-injection of LPS-GalN. Except for untreated mice (Control), each mouse received 0.5 ml of LPS-GalN (consisting of 100 ng of LPS plus 8 mg of D-galactosamine) i.p. The RJX dose levels (in ml/kg) are indicated in parentheses. Results are expressed as the mean and standard deviation Statistical significance between groups is shown by: ** P<0.001; *** P<0.001; **** P<0.0001 compared as LPS-GalN and ####P<0.0001 compared as LPS/GalN+NS group, ANOVA and Tukey's post-hoc test).

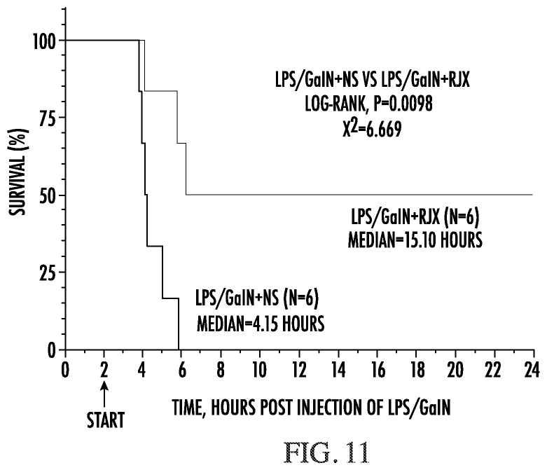

As shown in FIGS. 10A, 10B, and 10C, the serum IL-6 and TNF-α levels were markedly elevated in 6 of 6 control mice that were terminated at 2 hours after the i.p. LPS-GalN injection when compared to 6 untreated control mice. At the time of their termination, these LPS-GalN injected mice also had marked lipid peroxidation in the lungs as evidenced by dramatically elevated MDA levels in the lungs. Hence, at 2 hours post LPS-GalN, mice had evidence of a significant inflammatory response and oxidative stress in the lungs. In our proof of concept experiment aimed at evaluating RJX as a potential treatment for sepsis, we started treatments of mice with normal saline versus RJX at this time 2 h time point. Control mice (N=6) treated with 2 injections of vehicle (normal saline) at 2 hours and 3 hours, respectively, post LPS-GalN challenge all rapidly died within 4 hours at a median survival time of 2.15 hours after the first injection of normal saline and 4.15 hours after the LPS-GalN challenge. At the time of death, their serum IL-6 and TNF-α levels as well lung tissue MDA levels were drastically elevated and even higher than the levels in untreated control mice that were terminated at 2 hours post LPS-GalN injection (FIGS. 10A-10C). Notably, treatment of mice with 6-fold diluted 4.2 mL/kg RJX at 2 hours and 3 hours post LPS-GalN injection resulted in improved survival outcome with 3 of 6 mice remaining alive at 24 hours post LPS-GalN injection (FIG. 11).

Referring to FIG. 11, the median survival time of these mice was markedly longer than the median survival of normal saline-treated control mice (15.1 hours vs. 4.15 hours, P=0.0098). The serum levels of inflammatory cytokines IL-6 and TNF-α as well as lung MDA levels at the time of death (N=3) or lective termination at 24 hours (N=3) were lower than the baseline levels in mice sacrificed at the 2 hours post LPS-GalN challenge time point when RJX and normal saline treatments were initiated (FIGS. 10A, 10B, and 10C). These results provide direct evidence that low dose RJX is capable of preventing fatal cytokine storm and reduce the mortality rate of LPS/GalN-induced systemic inflammation when the treatment is delayed until after the onset of a systemic inflammatory cytokine response and oxidative stress in the lungs.

FIG. 11 illustrates In Vivo Protective Activity of Delayed-Onset RJX Treatments in the LPS-GalN Model of Sepsis, Systemic inflammation, Shock, ARDS and Multiorgan Failure. Groups of 6 BALB/C mice were treated with i.p injections of 6-fold diluted RJX (4.2 mL/kg, 0.5 ml/mouse) or vehicle (NS) 2 and 3 hours post-injection of LPS-GalN. Each mouse received 0.5 ml of LPS-GalN (consisting of 100 ng of LPS plus 8 mg of D-galactosamine) i.p. Percent (%) survival for each treatment group is shown as a function of time after the LPS-GalN challenge. Depicted are the survival curves for each group along with the median survival times and the log-rank P-value for the comparison of LPS-GalN+RJX group versus the LPS/GalN+NS group.

### Example 2.1—Clinical Safety of RJX

Rejuveinix (RJX) is a formulation of several vitamins, including ascorbic acid (Vitamin C), cyanocobalamin (Vitamin B12), thiamine hydrochloride (Vitamin B1), riboflavin 5′ phosphate (Vitamin B2), niacinamide (Vitamin B3), pyridoxine hydrochloride (Vitamin B6), calcium D-pantothenate, and magnesium sulphate as a potent calcium antagonist, representing components with reported, albeit controversial, protective activity in animal models of septic shock and ARDS as well as some of the clinical studies in septic patients. Increased lactate levels contribute to the enhanced mortality of septic patients. Two of the RJX ingredients, thiamine and magnesium sulfate, accelerate the lactate clearance, which has been shown to improve survival outcomes. While some preclinical, translational, early-stage, as well as late-stage clinical studies, have yielded promising positive data regarding the clinical impact potential of ascorbic acid, thiamine, riboflavin, niacinamide, and pyridoxine (Vit. B₆) in the prevention and treatment of cytokine storm, CRS, coagulopathy, ALI, acute kidney injury (AKI), ARDS, and MODS in the context of sepsis, other studies have not shown any meaningful activity. For example, Moskowitz et al. recently reported the results of a 205-patient, randomized blinded, multicenter study of ascorbic acid, thiamine and steroids in patients with septic shock that was performed at 14 centers in the U.S. Patients were randomly assigned to receive parenteral ascorbic acid (1500 mg), hydrocortisone (50 mg), and thiamine (100 mg) every 6 hours for 4 days (n=103) or placebo in matching volumes at the same time points (n=102). The primary endpoint was change in the Sequential Organ Failure Assessment (SOFA) score (range, 0-24; 0=best) between enrollment and 72 hours. There was no statistically significant interaction between time and treatment group with regard to SOFA score over the 72 hours after enrollment. Hence, the combination of ascorbic acid, corticosteroids, and thiamine, compared with placebo, did not result in a statistically significant reduction in SOFA score during the first 72 hours after enrollment. Similarly, Fujii et al. reported that treatment with intravenous vitamin C, hydrocortisone, and thiamine does not lead to a more rapid resolution of septic shock compared with intravenous hydrocortisone alone based on the results of a randomized trial in 216 patients with septic shock. In contrast to these studies, Iglesias et al. reported that the combination of IV ascorbic acid, thiamine, and hydrocortisone significantly reduced the time to resolution of shock based on another randomized study. Likewise, Byerly et al. reported that ascorbic acid plus thiamine is associated with increased survival in septic ICU patients based on the results of 11,330 patients with sepsis and elevated lactate levels regarding the impact of thiamine and ascorbic acid on survival outcome. After controlling for confounding factors, ascorbic acid (adjusted odds ratio [AOR], 0.69 [0.50-0.95]) and thiamine (AOR, 0.71 [0.55-0.93]) were independently associated with survival. Additional studies are needed to confirm these findings and assess any potential benefit from this treatment. RJX has no steroids and it contains niacinamide, pyridoxine, cyanocobalamine and Mg-sulfate in addition to ascorbic acid and thiamine. The clinical potential of RJX will be examined in COVID-19 patients with an emphasis on prevention of ARDS and multiorgan failure in COVID-19 patients at high risk for fatal viral sepsis rather than treatment of septic shock.

RJX is being developed as an anti-inflammatory and anti-oxidant treatment platform for patients with sepsis, including COVID-19 patients with viral sepsis and ARDS. Its clinical safety profile was examined in a clinical study.

Specifically, a Phase I, double-blind, placebo-controlled, randomized, two-part, ascending dose-escalation study was performed in participating 76 healthy volunteer human subjects in compliance with the ICH(E6) good clinical practice (GCP) guidelines to evaluate the safety, tolerability, pharmacokinetics, and pharmacodynamics of RJX. In the Phase I clinical study, RJX showed a very favorable safety profile and tolerability in human subjects. No participant developed serious adverse events (SAEs) or Grade 3-Grade 4 adverse events (AEs) or prematurely discontinued participation from the study.

The dose levels ranged from 0.024 mL/kg to 0.759 mL/kg in Part 1 and from 0.240 mL/kg to 0.759 mL/kg in Part 2. No deaths or SAEs were reported, none of the 39 RJX-treated subjects experienced Grade 3 or 4 AEs, and no AEs led to discontinuation of RJX.

In Part 2, no SAEs and no Grade 3 or 4 AEs were encountered by any of the 18 RJX-treated subjects. One subject in Cohort 1 developed a mild upper respiratory infection (URI); 3 subjects in Cohort 2 had mild infusion site discomfort, pain or reaction; and 3 subjects on Cohort 3 experienced mild-moderate pain (mild headache=1; mild pain in extremity=1, moderate back pain=1). The TEAEs in 2 of the subjects were considered possibly or probably related to RJX. Both patients were in Cohort 2: One patient had a mild infusion site discomfort on day 6 considered possibly related to RJX and another patient had a mild infusion site pain on days 6 and 7 considered probably related to RJX infusion. These TEAEs did not require additional treatment, and they did not cause discontinuation of the dosing or study. All AEs considered possibly or probably related to RJX infusions have recovered/resolved with no sequelae. There were no clinically significant abnormal findings in 12-lead safety ECGs, and no notable changes were detected compared with baseline. For both study parts, there were no clinically meaningful changes in laboratory values identified in observed values or mean changes from baseline compared with placebo or evaluated by increasing the RJX dose level. Based on its tolerability, the 0.500 ml/kg dose level was selected as the recommended Phase 2 dose (RP2D) level for future studies.

### Example 3—RJX in Combination with Dexamethasone Prevents Fatal Outcome in an Animal Model of Sepsis by Reversing Inflammatory Organ Injury

The experimental drug product RJX is being evaluated for its clinical impact potential for COVID-19-associated viral sepsis in a placebo-controlled randomized Phase I/II study. Here, we demonstrate that RJX, at a dose level that corresponds to less than 10% of its clinical maximum tolerated dose (MTD), exhibits potent anti-inflammatory activity in the preclinical LPS-GalN model of fatal sepsis. The combination of RJX plus dexamethasone (DEX) was more effective than RJX alone or DEX alone and (i) profoundly decreased the inflammatory cytokine responses to LPS-GalN (ii) mitigated the inflammatory tissue damage in the lungs and liver, and (iii) prevented a fatal outcome. Even when treatments were started after the onset of fulminant cytokine storm and systemic inflammation with severe lung damage, a near-complete recovery of the inflammatory lung injury was achieved within 24 hours. RJX may be an adjunct of standard of care in the multi-modality management of sepsis and its complications. The complications may be acute respiratory distress syndrome (ARDS) and multi-organ dysfunction (MOD).

Sepsis represents a strong systemic inflammatory response to an infection with a potentially fatal outcome due to its complications. Severe viral sepsis caused by SARS-CoV-2 shows a rapid progression associated with cytokine release syndrome (CRS) and a high case fatality rate in high-risk coronavirus disease 2019 (COVID-19) patients. The anti-sepsis drug Rejuveinix (RJX) exhibited promising single-agent anti-inflammatory activity in mice challenged with LPS-GalN. RJX showed a very favorable clinical safety and pharmacokinetics (PK) profile in a recently completed randomized, double-blind, placebo-controlled Phase I ascending dose-escalation study in healthy volunteers. No deaths, serious adverse events (SAEs), or Grade 3-4 adverse events (AEs) were observed in any of 57 healthy volunteers treated with RJX at dose levels ranging from 0.024 mL/kg to 0.759 mL/kg.

The primary objective of the present example was to compare the effects of RJX, the standard anti-inflammatory drug dexamethasone (DEX), and their combination on the severity of sepsis and survival outcome in an animal model that employs LPS-GalN to induce fatal sepsis. It was hypothesized that RJX, especially when combined with DEX, would improve the survival outcome of mice challenged with a lethal dose of LPS-GalN. This example demonstrates that RJX—at a dose level >10-fold lower than its clinical MTD—exhibits potent single-agent anti-inflammatory activity in the LPS-GalN model. The combination of RJX plus DEX immediately and profoundly decreased the inflammatory cytokine (IL-6, TNF-α) responses to LPS-GalN, mitigated the inflammatory tissue damage in the lungs and liver, and prevented a fatal outcome. Even when treatments were started after the onset of fulminant cytokine storm and systemic inflammation as well as very severe lung damage, a near-complete recovery of the inflammatory lung injury was achieved within 24 hours, and the survival outcome was improved.

It is anticipated that RJX in combination with other anti-inflammatory drugs as provided herein will lead to similar results.

### Example 3.1—Effectiveness of Treatment with RJX in Side by Side Comparison to Dexamethasone (DEX) in Reversing Acute Lung Injury and Acute Liver Injury in Mice Injected with LPS-GalN

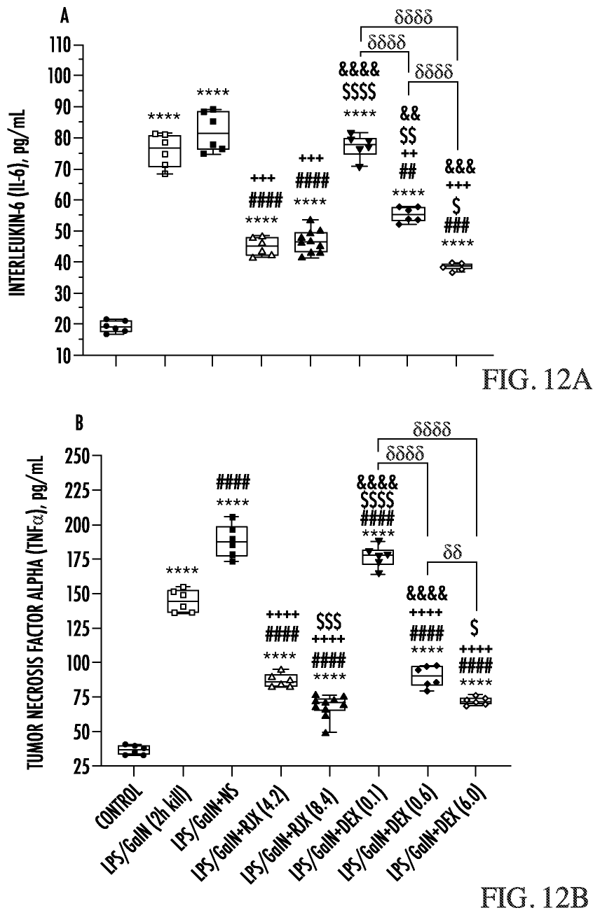

LPS-GalN-challenged mice experience a rapid onset systemic inflammation with markedly elevated inflammatory cytokine levels as well as severe lung injury as early as 2 hours after the administration of LPS-GalN. It was first set out to determine the effects of RJX vs. DEX on the LPS-GalN induced inflammatory cytokine response in BALB/c mice. FIG. 12A illustrates effects on interleukin 6 (IL-6; FIG. 12A), and FIG. 12B illustrates effects on tumor necrosis factor-alpha (TNF-α) in a Mouse Model of Fatal Cytokine Storm, Sepsis, Systemic Inflammation, ARDS and Multiorgan Failure. BALB/C mice were treated with i.p injections of RJX (0.7 ml/kg=6-fold diluted, 4.2 ml/kg, 0.5 ml/mouse; or 1.4 ml/kg=6-fold diluted 8.4 ml/kg, 0.5 ml/mouse), DEX (0.1 mg/kg, 0.6 mg/kg and 6.0 mg/kg), or vehicle (NS, 0.5 mL/mouse) two hours post-injection of LPS-GalN, and only LPS-GalN for 2 hours. Except for untreated control mice (Control), each mouse received 0.5 ml of LPS-GalN (consisting of 100 ng of LPS plus 8 mg of D-galactosamine i.p.). A group of 6 control mice received LPS-GalN and then electively terminated at 2 hours. The depicted Whisker plots represent the median and values for serum IL-6 and TNF-α levels from all 6 mice from each group except for the 1.4 ml/kg RJX group where blood samples were obtained from all 10 mice. In FIG. 12A, Welch's ANOVA and Tamhane's T2 post-hoc test were used for comparing the results among different treatment groups. In FIG. 12B, ANOVA and Tukey post-hoc test were used for comparing the results among different treatment groups. Statistical significance between groups is shown by **** p<0.0001 as compared to control group, and ##p<0.01; ###p<0.001; ####p<0.0001 as compared to LPS/GalN (2h kill) group, ++p<0.01; +++p<0.001; ++++p<0.0001 as compared to LPS/GalN+NS group, $ p<0.05; $$ p<0.01; $$$ p<0.001; $$$$ p<0.0001 LPS/GalN+RJX (4.2) group, && p<0.01; &&& p<0.001; &&&& p<0.0001 as compared to LPS/GalN+RJX (8.4) group, and and δδ p<0.01; δδδδp<0.0001 pairwise comparison. At 2 hours post-injection of LPS-GalN, the serum IL-6 and TNF-α levels were markedly elevated in electively terminated control mice consistent with an inflammatory cytokine response. Low dose RJX at both 0.7 ml/kg (HED: 0.057 ml/kg; 7.5% of human MTD) and 1.4 ml/kg (HED: 0.114 ml/kg; 15% of human MTD) dose levels administered at 2 h after LPS-GalN injection effectively reversed the LPS-GalN induced increased serum levels of the pro-inflammatory cytokines IL-6 and TNF-α within 24 h. At both of these low dose levels, RJX was significantly more effective than 0.1 mg/kg DEX (HED: 0.008 mg/kg; 0.65 mg for an 80 kg person) or 0.6 mg/kg DEX (HED: 0.05 mg/kg; 4 mg standard dose for an 80 kg person) in reducing the IL-6 levels. 1.4 ml/kg low dose (15% of MTD) RJX was as effective as 6.0 mg/kg supra-therapeutic high dose DEX (HED: 0.49 mg/kg; 39 mg dose for an 80 kg person, which is 4.9-9.8 fold higher than the standard 4-8 mg dose levels for DEX) in reducing the TNF-α levels and slightly less effective in reducing the IL-6 levels. By comparison, treatment with NS that was included as vehicle control did not reverse the fulminant cytokine response or prevent its progression.

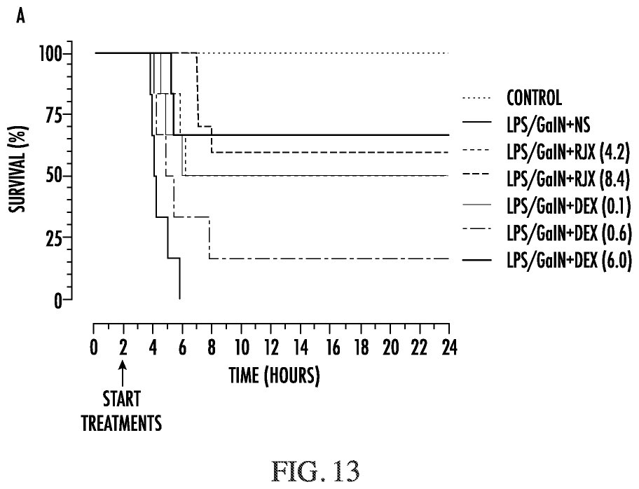

The observed reversal of the inflammatory cytokine response by RJX was associated with a significant improvement of the survival outcome in this LPS-GalN model of sepsis. Notably, 0.7 ml/kg low dose RJX was moderately more effective than DEX at a 0.1 mg/kg low dose level (Median survival: 15.1 h vs. 5.1 h; 24-h mortality: 50% vs. 83.3%), and it was as effective as DEX at the standard dose 0.6 mg/kg. See FIG. 13, which illustrates results for in vivo treatment activity of RJX and the different doses of DEX in the LPS-GalN Mouse Model of Fatal Cytokine Storm, Sepsis, Systemic Inflammation, ARDS and Multiorgan Failure. BALB/C mice were treated with i.p injections of RJX (4.2 mL/kg or 8.4 mL/kg, 0.5 ml/mouse), DEX (0.1 mg/kg, 0.6 mg/kg and 6.0 mg/kg 0.5 mL/mouse), or vehicle (NS, 0.5 mL/mouse) two hours post-injection of LPS-GalN. Except for untreated control mice (Control), each mouse received 0.5 ml of LPS-GalN (consisting of 100 ng of LPS plus 8 mg of D-galactosamine i.p.). The cumulative proportion of mice remaining alive (Survival, %) is shown as a function of time after the LPS-GalN challenge. FIG. 13 shows the Kaplan Meier survival curves. The below tables provide survival data with statistical analysis of the different treatment groups.

| Survival Analysis | Censored | Events | n | Proportion | Median | |
|---|---|---|---|---|---|---|
| Control | 6 | 0 | 6 | 0.0% | ≥24 | h |
| LPS/GaIN + NS | 0 | 6 | 6 | 100.0% | 4.2 | h |
| LPS/GaIN + RJX | 3 | 3 | 6 | 50.0% | 15.1 | h |
| (4.2) | | | | | | |
| LPS/GaIN + RJX | 6 | 4 | 10 | 40.0% | ≥24 | h |
| (8.4) | | | | | | |
| LPS/GaIN + DEX | 1 | 5 | 6 | 83.3% | 5.1 | h |
| (0.1) | | | | | | |
| LPS/GaIN + DEX | 3 | 3 | 6 | 50.0% | 15.0 | h |
| (0.6) | | | | | | |
| LPS/GaIN + DEX | 4 | 2 | 6 | 33.3% | ≥24 | h |
| (6.0) | | | | | | |

| | | Log-rank | | p value |
|---|---|---|---|---|
| Pairwise comparisons | | Chi-square | p value | summary |
| LPS/GaIN + NS | LPS/GaIN + RJX(4.2) | 6.669 | 0.0098 | ** |
| LPS/GaIN + NS | LPS/GaIN + RJX(8.4) | 19.259 | 0.0001 | **** |
| LPS/GaIN + NS | LPS/GAIN + DEX (0.1) | 2.401 | 0.1213 | ns |
| LPS/GaIN + NS | LPS/GAIN + DEX (0.6) | 6.066 | 0.0138 | * |
| LPS/GaIN + NS | LPS/GAIN + DEX (6.0) | 8.044 | 0.0046 | ** |
| LPS/GaIN + RJX(4.2) | LPS/GAIN + DEX (0.1) | 1.378 | 0.2405 | ns |
| LPS/GaIN + RJX(4.2) | LPS/GAIN + DEX (0.6) | 0.004 | 0.9467 | ns |
| LPS/GaIN + RJX(4.2) | LPS/GAIN + DEX (6.0) | 0.193 | 0.6606 | ns |
| LPS/GaIN + RJX(8.4) | LPS/GaIN + DEX (0.1) | 5.288 | 0.0215 | * |
| LPS/GaIN + RJX(8.4) | LPS/GaIN + DEX (0.6) | 0.633 | 0.4264 | ns |
| LPS/GaIN + RJX(8.4) | LPS/GaIN + DEX (6.0) | 0.001 | 0.9991 | ns |
| LPS/GaIN + DEX (0.1) | LPS/GaIN + DEX (0.6) | 1.220 | 0.2694 | ns |
| LPS/GaIN + DEX (0.1) | LPS/GaIN + DEX (6.0) | 2.848 | 0.0915 | ns |

Notably, at a dose level of 1.4 ml/kg, which corresponds to 15% of its clinical MTD, RJX reduced the mortality to 40% (Median survival >2 4h). These results were very similar to and statistically not different from the 33.3% mortality (Median survival >2 4h) (P=0.99) achieved with DEX at the supratherapeutic 6.0 mg/kg dose level that is 4.9-9.8 fold higher than the standard 4-8 mg clinically applied dose levels for DEX (FIG. 13).

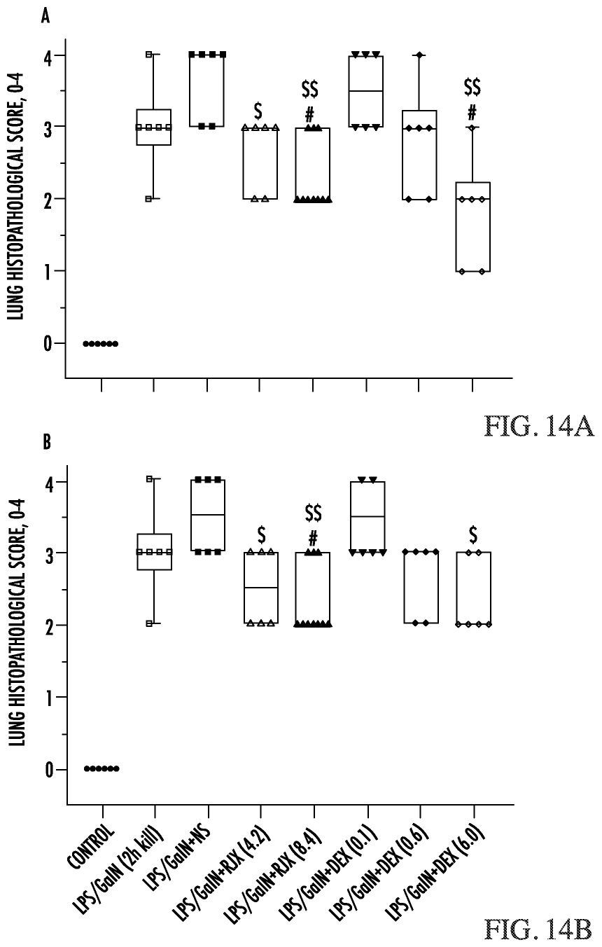

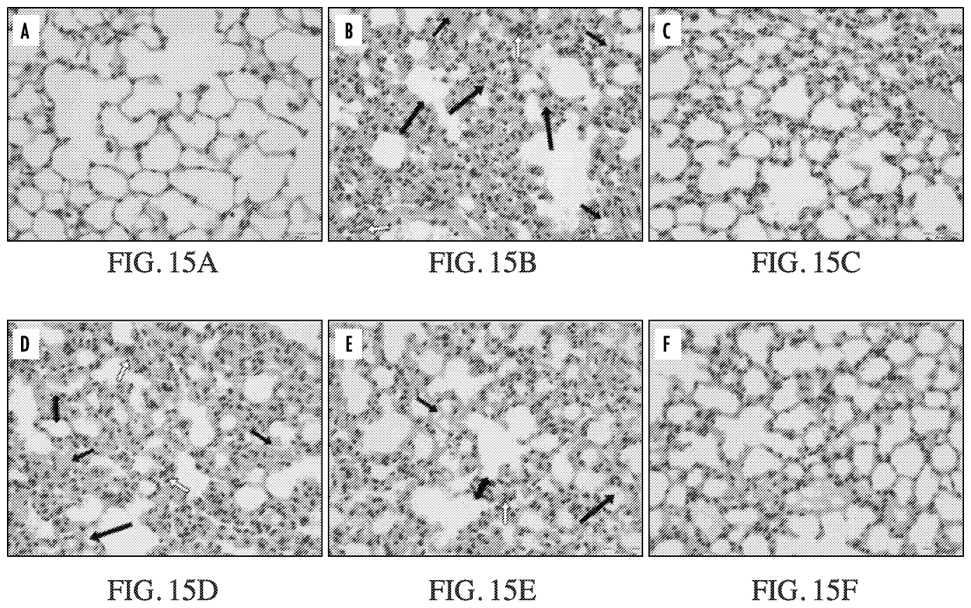

Referring to FIGS. 14A, 14B, and 15A-15F, as evidenced in FIG. 14A and FIGS. 15A-15F, RJX at 0.7 ml/kg (Mean±SE ALI score: 2.7±0.2) or 1.4 ml/kg (Mean±SE ALI score: 2.3±0.2) low dose levels as well as DEX at the 0.6 mg/kg (HED: 0.05 mg/kg; 4 mg standard dose for an 80 kg person) (Mean±SE ALI score: 2.8±0.3) dose level (but not DEX at 0.1 mg/kg dose level; Mean±SE ALI score: 3.5±0.2) were capable of partially reversing the lung injury that was documented at 2 h post LPS-GalN injection when treatments were initiated (Mean±SE ALI score: 3.0±0.3), as measured by the lung histopathological scores (i.e., acute lung injury [ALI] scores).

FIGS. 14A and 14B illustrate tissue-level in vivo activity of RJX and the different doses of DEX, treatments on Lung and Liver Histopathological Scores in a Mouse Model of Fatal Cytokine Storm, Sepsis, Systemic Inflammation, ARDS and Multiorgan Failure. BALB/C mice were treated with i.p injections of RJX (6-fold diluted, 4.2 mL/kg or 8.4 mL/kg, 0.5 ml/mouse), DEX (0.1 mg/kg, 0.6 mg/kg and 6.0 mg/kg), or vehicle (NS, 0.5 mL/mouse) two hours post-injection of LPS-GalN, and only LPS-GalN for 2 hours. Except for untreated control mice (Control), each mouse received 0.5 ml of LPS-GalN (consisting of 100 ng of LPS plus 8 mg of D-galactosamine i.p.). The depicted Whisker plots represent the median and values. In (A), the lung histopathological score (“lung injury score”) was graded according to a 5-point scale from 0 to 4 as follows: 0, I, 2, 3, and 4 represented no damage, mild damage, moderate damage, severe damage, and very severe damage, respectively. In (B), the liver histopathological score (“liver injury score”) was graded according to a 5-point scale from 0 to 4 as follows: 0, I, 2, 3, and 4 represented no damage, mild damage, moderate damage, severe damage, and very severe damage, respectively. Kruskal-Wallis test and Mann Whitney U test were used for comparing the results among different treatment groups Statistical significance between groups is shown by #p<0.05; as compared to LPS/GalN (2h kill) group, and $ p<0.05; $$ p<0.01; as compared to LPS/GalN+NS group.

FIGS. 15A-15F illustrate the effects of RJX and the different doses of DEX, treatments on acute lung injury and inflammation in a mouse model of fatal cytokine storm, sepsis, systemic inflammation, ARDS and Multiorgan Failure. Groups of 6 BALB/C mice were treated with i.p injections of RJX (6-fold diluted, 4.2 mL/kg, 0.5 ml/mouse), DEX (0.1 mg/kg, 0.6 mg/kg, and 6.0 mg/kg, 0.5 mL/mouse), or vehicle (NS, 0.5 mL/mouse) two hours post-injection of LPS-GalN. Except for untreated control mice (FIG. 15A), each mouse received 0.5 ml of LPS-GalN (consisting of 100 ng of LPS plus 8 mg of D-galactosamine i.p.). The lung histopathological ALI scores were 0 for each of the control mice (FIG. 15A), ranged from 3 to 4 (Median: 4) for the LPS-GalN+NS group (FIG. 15B), from 2-3 (Median: 3) for LPS-GalN+RJX (0.7 ml/kg) group (FIG. 15C), from 3-4 (Median: 3.5) for LPS-GalN+DEX (0.1 mg/kg) (FIG. 15D), from 2-4 (Median: 3) for LPS-GalN+DEX (0.6 mg/kg) group (FIG. 15E) and from 1-3 (Median: 2) for LPS-GalN+DEX (6.0 mg/kg) group (FIG. 15F). Depicted are microscopic images of lung tissues of representative mice from the untreated control group and various treatment groups. White arrow: inflammatory cell infiltration; Black arrow (short): Exudate, edema; Black arrow (Long): hemorrhage; Black double-headed arrow: the thickness of the alveolar wall. H&E ×400.

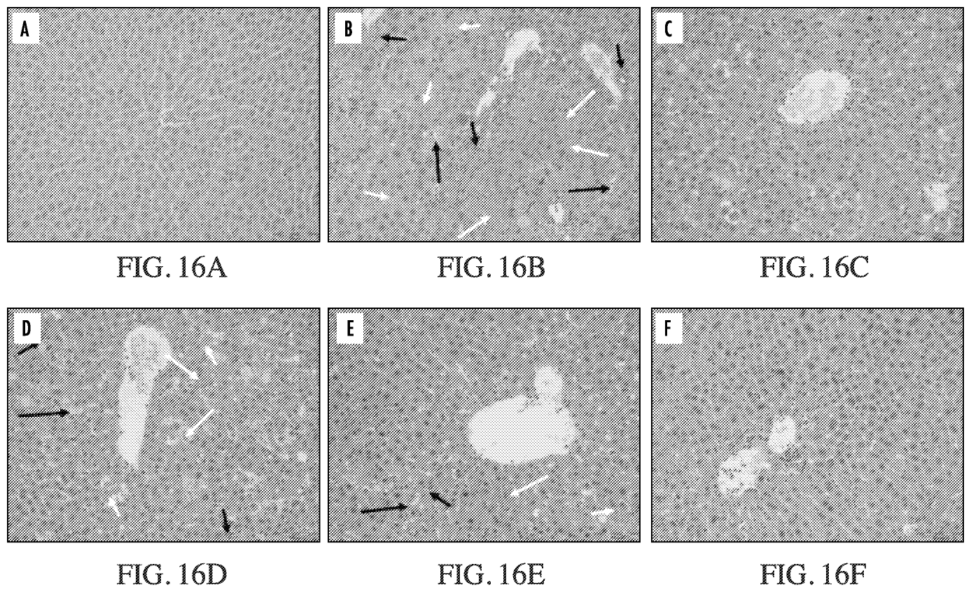

FIGS. 16A-16F illustrate the effects of RJX and the different doses of the DEX, treatments on liver injury and inflammation in a mouse model of fatal cytokine storm, sepsis, systemic inflammation, ARDS and Multiorgan Failure. Groups of 6 BALB/C mice were treated with i.p injections of RJX (6-fold diluted, 4.2 mL/kg, 0.5 ml/mouse), DEX (0.1 mg/kg, 0.6 mg/kg, and 6.0 mg/kg, 0.5 mL/mouse), or vehicle (NS, 0.5 mL/mouse) two hours post-injection of LPS-GalN. Except for untreated control mice (FIG. 16A), each mouse received 0.5 ml of LPS-GalN (consisting of 100 ng of LPS plus 8 mg of D-galactosamine i.p.). The liver histopathological scores ranged from 3 to 4 for the LPS-GalN+NS group (FIG. 16B), from 2-3 for LPS-GalN+RJX group (FIG. 16C), from 3-4 for LPS-GalN+DEX (0.1) (FIG. 16D), from 2-3 for LPS-GalN+DEX (0.6) group (FIG. 16E) and from 2-3 for LPS-GalN+DEX (6.0) group (FIG. 16F). Depicted are microscopic images of the liver tissues of representative mice from the untreated control group and various treatment groups. While the liver histopathological score median was 0 for untreated control mouse, the liver score median for the depicted mice treated with 0.6 mg/kg or 6.0 mg/kg DEX were 3 for each, and the liver score median for the LPS-GalN+NS treated control mice was 3.5. Black arrow (short): Inflammatory cell infiltration; Black arrow (long): Congestion; White arrow (long): Necrosis; White arrow (short): Hydropic degeneration. H&E 200×.

The best results were obtained with 6.0 mg/kg supra-therapeutic high dose DEX (HED: 0.49 mg/kg; 39 mg dose for an 80 kg person) (Mean±SE ALI score: 1.8±0.3; FIG. 14A). By comparison, the lung damage further progressed in control mice treated with NS (vehicle) (Mean±SE ALI score: 3.7±01). Similar to its effects on the LPS-GalN induced ALI, 0.7 ml/kg or 1.4 ml/kg low dose RJX as well as standard 0.6 mg/kg and very high 6.0 mg/kg dose levels of DEX (but not DEX at 0.1 mg/kg dose level) significantly reduced the liver injury (FIG. 14A; FIGS. 16A-F). The histopathological liver damage scores (Mean±SE) were 0±0 for control mice not challenged with LPS-GalN, 3.5±0.3 for mice electively terminated 2 h post LPS-GalN, 3.5±0.2 for mice treated with NS post LPS-GalN, 3.3±0.2 for 0.1 mg/kg DEX, 2.7±0.2 for 0.6 mg/kg DEX, 2.3±0.2 for 6.0 mg/kg DEX, 2.5±0.2 for 0.7 ml/kg RJX, and 2.3±0.2 for 1.4 ml/kg RJX.

### Example 3.2—Effectiveness of Treatment with RJX in Combination with Dexamethasone (DEX) in Reversing Fatal Cytokine Storm, Acute Lung Injury and Acute Liver Injury in Mice Injected with LPS-GalN

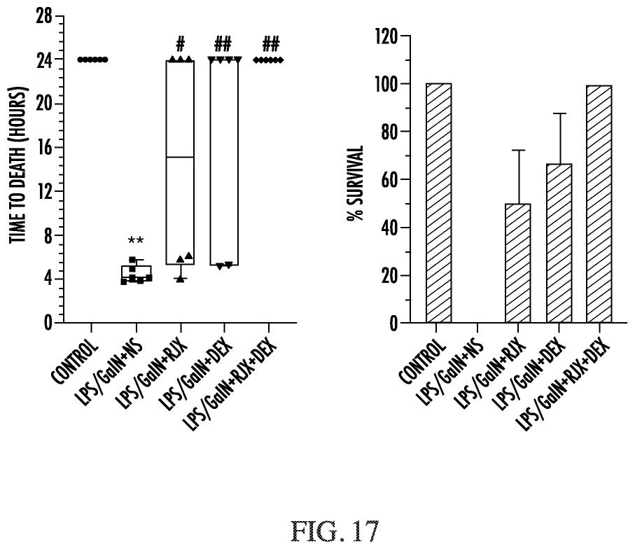

As there was significant residual tissue damage in the lungs and liver of surviving LPS-GalN challenged mice treated with RJX or DEX (even at the supra-therapeutic dose 6.0 mg/kg dose level), it was next sought to determine if a combination of low dose RJX (0.7 ml/kg) and supratherapeutic high dose DEX (6.0 mg/kg) could improve the survival outcome and most importantly the tissue healing after LPS-GalN exposure. Treatments were initiated at 2 h after LPS-GalN injection at a time of documented active systemic inflammation. The combination therapy was more effective than RJX alone or DEX alone (FIG. 17).

FIG. 17 illustrates therapeutic use of low dose RJX+Supratherapeutic high dose DEX combination after onset of systemic inflammation and lung injury improves the survival outcome in the LPS-GalN Mouse Model of Fatal Cytokine Storm and Sepsis. Groups of 6 BALB/C mice were treated with i.p injections of RJX (6-fold diluted, 4.2 mL/kg, 0.5 ml/mouse), DEX (6 mg/kg, 0.5 mL/mouse), RJX+DEX (0.5 mL/mouse), or vehicle (NS, 0.5 mL/mouse) two hours post-injection of LPS-GalN. Except for untreated control mice (Control), each mouse received 0.5 ml of LPS-GalN (consisting of 100 ng of LPS plus 8 mg of D-galactosamine) i.p. The depicted Whisker plots represent the median and values for time to death from 6 mice in each group. Kruskall Wallis and Mann Whitney U (pairwise comparisons test) test were used for comparing the results among different treatment groups. Statistical significance between groups is shown by: ** p<0.01 compared as control group and, #p<0.05; ##p<0.01 compared as LPS/GalN+NS group.

In contrast to the rapid death of all control mice treated with NS (Median survival: 4.3 h post LPS-GalN or 2.3 h after administration of NS), 100% of mice treated with RJX+DEX survived the LPS-GalN challenge (Median survival: >24 hours post LPS-GalN or >22 hours after initial administration of RJX+DEX) (FIG. 17). By comparison, the combined group of mice treated with monotherapy (i.e., RJX alone or DEX alone) (n=12) had a 24-hour survival rate of 41.7% (Monotherapy with RJX or DEX vs. Combination therapy with RJX+DEX: Log-rank X²=3.053, p=0.081).

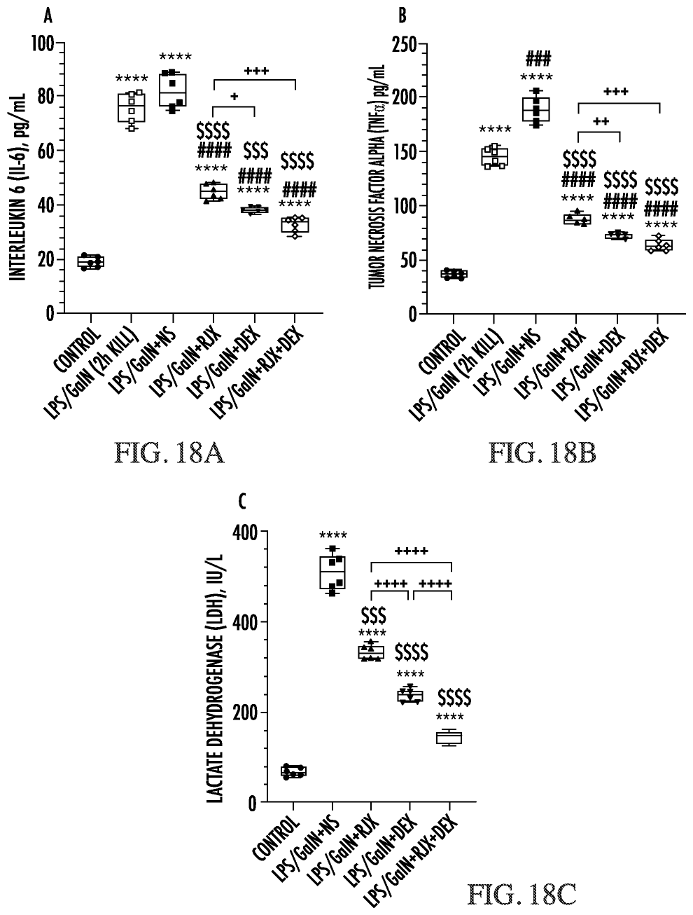

RJX+DEX effectively reversed the increased serum levels of the systemic inflammation markers (IL6, TNF-α, and LDH) within 24 h, and it appeared to be overall more effective than DEX alone or RJX alone (FIGS. 18A, 18B, and 18C).

FIGS. 18A-18C illustrate therapeutic use of low dose RJX plus supratherapeutic high dose DEX combination after onset of systemic inflammation and lung injury reverses inflammatory cytokine response and systemic inflammation in the LPS-GalN Mouse Model of Fatal Cytokine Storm and Sepsis. Depicted are the effects of Rejuveinix (RJX), Dexamethasone (DEX), and RJX+DEX combination treatments on serum levels of interleukin 6 (IL-6; FIG. 18A), tumor necrosis factor-alpha (TNF-α; FIG. 18B), and lactate dehydrogenase (LDH; FIG. 18C). Groups of 6 BALB/C mice were treated with i.p injections of RJX (6-fold diluted, 4.2 mL/kg, 0.5 ml/mouse), DEX (6 mg/kg, 0.5 mL/mouse), RJX+DEX (0.5 ml/mouse), or vehicle (NS, 0.5 ml/mouse) two hours post-injection of LPS-GalN. Except for untreated control mice (Control), each mouse received 0.5 ml of LPS-GalN (consisting of 100 ng of LPS plus 8 mg of D-galactosamine) i.p. The depicted Whisker plots represent the median and values. Welch's ANOVA and Tamhane's T2 post-hoc test was used for comparing the results among different treatment groups. Statistical significance between groups is shown by **** p<0.0001 as compared to control group, ###p<0.001; ####p<0.0001 as compared to LPS/GalN (2h kill) group, $$$ p<0.001; $$$$ p<0.0001 as compared to LPS/GalN+NS, and +p<0.05; ++p<0.01; +++p<0.001; ++++p<0.0001 pairwise comparisons between the groups.

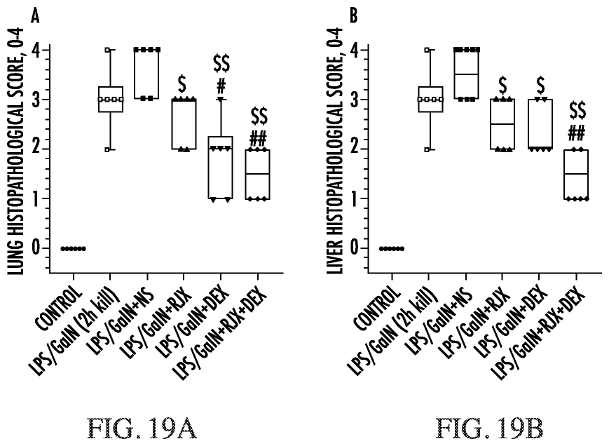

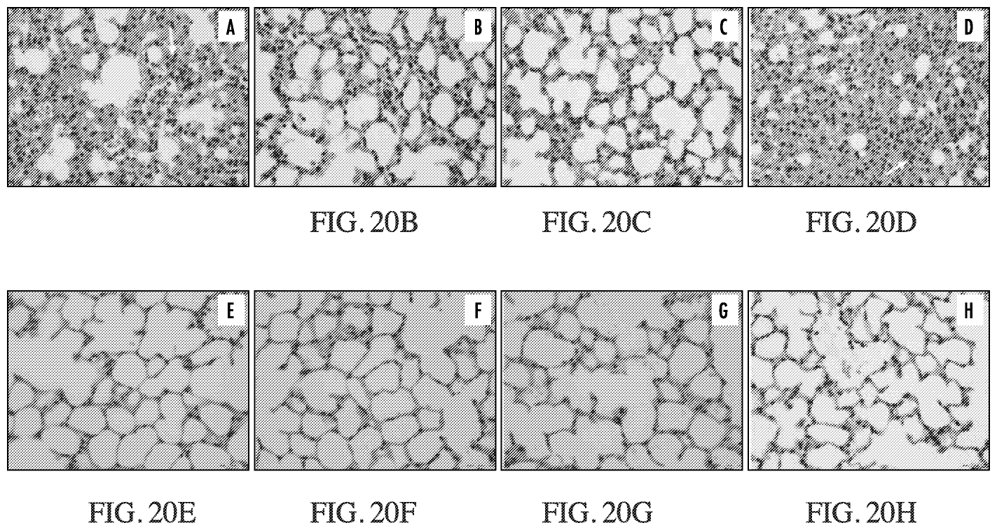

The serum levels of LDH, a biomarker of systemic inflammation and tissue damage as significantly lower in mice treated with the RJX+DEX combination than mice treated with RJX alone (p<0.0001) or DEX alone (p<0.0001) (FIGS. 18A, 18B, and 18C). Notably, delayed treatments with RJX, DEX or RJX+DEX starting at 2 h after the LPS-GalN injection partially reversed the lung damage as evidenced by significantly reduced histopathological lung scores (FIGS. 19A and 19B, FIG. S5). The tissue healing activity of the combination was more pronounced than the tissue healing activity of RJX alone or DEX alone (FIGS. 19A and 19B, FIGS. 20A-20H).

FIGS. 19A-19B illustrate in vivo treatment activity of low dose RJX, supratherapeutic high dose DEX and their combination on lung and liver histopathological scores in the LPS-GalN mouse model of fatal cytokine storm and sepsis. Groups of 6 BALB/C mice were treated with i.p injections of RJX (6-fold diluted, 4.2 mL/kg, 0.5 ml/mouse), DEX (6.0 mg/kg, 0.5 mL/mouse), or vehicle (NS, 0.5 mL/mouse) two hours post-injection of LPS-GalN. Except for untreated control mice (Control), each mouse received 0.5 ml of LPS-GalN (consisting of 100 ng of LPS plus 8 mg of D-galactosamine) i.p. In FIG. 19A, the lung histopathological score (“lung injury score”) was graded according to a 5-point scale from 0 to 4 as follows: 0, 1, 2, 3, and 4 represented no damage, mild damage, moderate damage, severe damage, and very severe damage, respectively. In FIG. 19B, the liver histopathological score (“liver injury score”) was graded according to a 5-point scale from 0 to 4 as follows: 0, I, 2, 3, and 4 represented no damage, mild damage, moderate damage, severe damage, and very severe damage, respectively. Mann Whitney U test were used for comparing the results among different treatment groups Statistical significance between groups is shown by #p<0.05; ##p<0.01 as compared to LPS/GalN (2h kill) group, $ p<0.05; $$ p<0.01 as compared to LPS/GalN+NS group.

FIGS. 20A-20H illustrate RJX plus DEX Combination Mitigates Acute Lung Injury and Inflammation in a Mouse Model of Fatal Cytokine Storm and Sepsis. FIG. 20A: Lung tissue of a representative mouse injected with LPS-GalN without any pre- or post-LPS-GalN treatments and electively sacrificed at 2 hours to confirm the rapid onset of lung damage. The histopathological ALI score was 3 consistent with severe lung damage. Yellow arrow: inflammatory cell infiltration; blue arrow: exudate; orange arrow: hemorrhage; green block: thickness of alveolar wall. FIG. 20B: Lung tissue of a LPS-GalN injected representative mouse treated with a single dose of RJX at 2 hours post-LPS-GalN. The mouse was electively sacrificed at 24 hours post LPS-GalN. ALI score=2 (moderate lung damage). FIG. 20C: Lung tissue of a LPS-GalN injected representative mouse treated with a single dose of DEX at 2 hours post-LPS-GalN. The mouse was electively sacrificed at 24 hours post LPS-GalN. ALI score=2 (moderate lung damage). FIG. 20D: Lung tissue of a LPS-GalN injected representative mouse treated with a single dose of NS at 2 hours post-LPS-GalN. The mouse died of sepsis at 4.2 hours post LPS-GalN. ALI score=4 (very severe lung damage). Yellow arrow: inflammatory cell infiltration; blue arrow: exudate; orange arrow: hemorrhage; green block: thickness of alveolar wall. FIG. 20E: Lung tissue of a healthy control mouse not injected with LPS-GalN and electively sacrificed at 24 hours. ALI score=0 (no lung damage). FIG. 20F and FIG. 20G: Lung tissues from two representative LPS-GalN injected control mice treated RJX+DEX 2 hours before at 2 hours post-LPS-GalN. These mice survived the LPS-GalN challenge and were electively sacrificed at 24 hours. No lung damage was detected (Histopathological lung score/ALI score=0). FIG. 20H: Lung tissue of a LPS-GalN injected representative mouse treated with RJX+DEX at 2 hours post-LPS-GalN. The mouse was electively sacrificed at 24 hours post LPS-GalN. ALI score=1 (mild lung damage). H&E X400.

The lung histopathological ALI scores ranged from 3 to 4 for the LPS-GalN +NS, from 2-3 for the LPS-GalN+RJX, from 1-3 for LPS-GalN+DEX, and from 1-2 for LPS-GalN+RJX+DEX. Hence, even when treatments were delayed until the onset of fulminant cytokine storm and systemic inflammation with severe oxidative stress as well as very severe lung damage, a near-complete recovery of the inflammatory lung injury was achieved within 24 h. Similar to its effects on the LPS-GalN induced ALI, RJX+DEX combination significantly reduced the liver injury (FIGS. 19A and 19B). While 12 of 12 mice (100%) treated with either RJX alone (N=6) or DEX alone (N=6) had moderate to severe residual damage in either their lungs or liver, 2 of the 6 mice (33.3%) treated with the combination regimen had no or minimal damage (i.e., histopathological damage scores: 0-1) in both organs (p=0.098, Fisher's exact test).

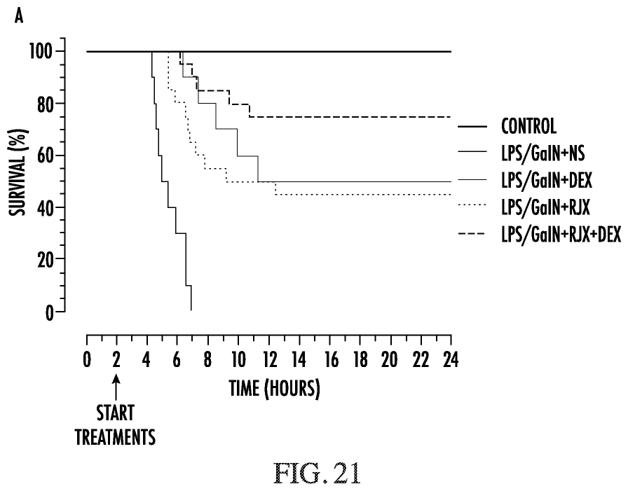

The efficacy of a combination of low dose RJX (0.7 ml/kg) and standard dose DEX (0.6 mg/kg) was next evaluated. Treatments were initiated at 2 h after LPS-GalN injection at a time of documented active systemic inflammation. The outcome results obtained with DEX alone or RJX alone were inferior to those obtained with the RJX+DEX combination. In contrast to the rapid death of all control mice treated with NS (Median survival: 5.1 h post LPS-GalN or 3.1 h after administration of NS), 15 of 20 (75%) of mice treated with RJX+DEX survived the LPS-GalN challenge (Median survival: >24 h post LPS-GalN (FIG. 21).

FIG. 21 illustrates in vivo treatment activity of low dose RJX, standard dose DEX, and their combination in the LPS-GalN mouse model of fatal cytokine storm, sepsis, systemic inflammation, ARDS and Multiorgan Failure. BALB/C mice were treated with i.p injections of RJX (n=20, 6-fold diluted, 4.2 mL/kg, 0.5 ml/mouse), DEX (n=10, 0.6 mg/kg, 0.5 mL/mouse), RJX+DEX (n=20, 0.5 mL/mouse), or vehicle (NS, 0.5 mL/mouse) two hours post-injection of LPS-GalN. Except for untreated control mice (Control, n=10), each mouse received 0.5 ml of LPS-GalN (consisting of 100 ng of LPS plus 8 mg of D-galactosamine) i.p. The cumulative proportion of mice remaining alive (Survival, %) is shown as a function of time after the LPS-GalN challenge. FIG. 21 shows the Kaplan Meier survival curves. Survival data with statistical analysis of the different treatment groups is shown in the below tables.

| Survival Analysis | Censored | Events | n | Proportion | Median | |
|---|---|---|---|---|---|---|
| Control | 10 | 0 | 10 | 0.0% | ≥24 | h |
| LPS/GaIN + NS | 0 | 10 | 10 | 100.0% | 5.1 | h |
| LPS/GaIN + DEX | 5 | 5 | 10 | 50.0% | 17.6 | h |
| LPS/GaIN + RJX | 11 | 9 | 20 | 45.0% | 10.8 | h |
| LPS/GaIN + | 15 | 5 | 20 | 25.0% | ≥24 | h |
| DEX + RJX | | | | | | |

| | | Log-rank | | p value |
|---|---|---|---|---|
| Pairwise comparisons | | Chi-square | p value | summary |
| Control | LPS/GaIN + | 21.837 | 0.0001 | **** |
| | NS | | | |
| Control | LPS/GaIN + | 6.389 | 0.0115 | * |
| | DEX | | | |
| Control | LPS/GaIN + | 7.624 | 0.0947 | ** |
| | RJX | | | |
| Control | LPS/GaIN + | 2.793 | 0.0040 | ** |
| | DEX + RJX | | | |
| LPS/GaIN + NS | LPS/GaIN + | 18.258 | 0.0001 | **** |
| | DEX | | | |
| LPS/GaIN + NS | LPS/GaIN + | 20.151 | 0.0001 | **** |
| | RJX | | | |
| LPS/GaIN + NS | LPS/GaIN + | 36.369 | 0.0001 | **** |
| | DEX + RJX | | | |
| LPS/GaIN + DEX | LPS/GaIN + | 0.313 | 0.5759 | ns |
| | RJX | | | |
| LPS/GaIN + DEX | LPS/GaIN + | 1.583 | 0.2083 | ns |
| | DEX + RJX | | | |
| LPS/GaIN + RJX | LPS/GaIN + | 4.365 | 0.0367 | * |
| | DEX + RJX | | | |
| Monotherapy DEX | LPS/GaIN + | 3.977 | 0.0461 | * |
| or RJX | DEX + RJX | | | |

By comparison, the combined group of mice treated with monotherapy (i.e., RJX alone or DEX alone) (N=30) had a 24-h survival rate of 53% (i.e., 16 of 30 mice) (Monotherapy with RJX or DEX vs. Combination therapy with RJX+DEX: Log-rank X²=3.977, p=0.046).

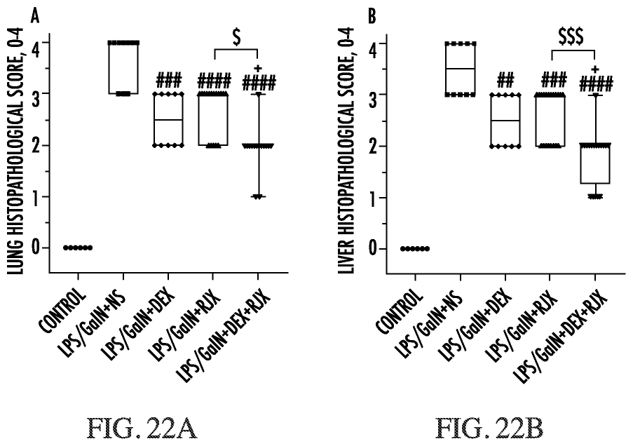

Notably, delayed treatments with RJX+DEX starting at 2 h after the LPS-GalN injection reversed the lung damage, as evidenced by significantly reduced histopathological lung scores (FIGS. 22A and 22B).

FIGS. 22A and 22B illustrate in vivo treatment activity of RJX, DEX, and RJX+DEX on lung and Liver Histopathological Scores in the LPS-GalN Mouse Model of Fatal Cytokine Storm, Sepsis, Systemic Inflammation, ARDS and Multiorgan Failure. BALB/C mice were treated with i.p injections of RJX (n=20, 6-fold diluted, 4.2 mL/kg, 0.5 ml/mouse), DEX (n=10, 6 mg/kg, 0.5 mL/mouse), RJX+DEX (n=20, 0.5 mL/mouse), or vehicle (NS, 0.5 mL/mouse) two hours post-injection of LPS-GalN. Except for untreated control mice (Control, n=10), each mouse received 0.5 ml of LPS-GalN (consisting of 100 ng of LPS plus 8 mg of D-galactosamine) i.p. The depicted Whisker plots represent the median and values. In (A), the lung histopathological score (“lung injury score”) was graded according to a 5-point scale from 0 to 4 as follows: 0, I, 2, 3, and 4 represented no damage, mild damage, moderate damage, severe damage, and very severe damage, respectively. In (B), the liver histopathological score (“liver injury score”) was graded according to a 5-point scale from 0 to 4 as follows: 0, 1, 2, 3, and 4 represented no damage, mild damage, moderate damage, severe damage, and very severe damage, respectively. Kruskal-Wallis test and Mann Whitney U test were used for comparing the results among different treatment groups Statistical significance between groups is shown by ##p<0.01; ###p<0.001; ####p<0.0001 as compared to LPS/GalN+NS group, +p<0.05 as compared to LPS/GalN+DEX group, and $ p<0.05; $$$ p<0.001 pairwise comparisons between the groups. In (C), for severe damage, lung and liver histological scores were compared by Pearson's Chi-Square or Fisher's Exact test. a Fisher's Exact Chi-Square test used.

The tissue healing activity of the combination was more pronounced than the tissue healing activity of RJX alone or DEX alone (FIG. 22A). Hence, even when treatments were delayed until the onset of fulminant cytokine storm and systemic inflammation as well as very severe lung damage, a near-complete recovery of the inflammatory lung injury was achieved in the vast majority of mice within 24 hours. Similar to its effects on the LPS-GalN induced ALI, RJX+DEX combination significantly reduced the liver injury (FIG. 22B). While 5 of 10 mice (50%) treated with standard dose DEX alone and 14 of 20 mice (70%) treated with low dose RJX alone had severe residual lung injury (histopathological lung score >3), only 2 of 20 mice (10%) of mice treated with the DEX+RJX combination had severe residual lung damage. The observed superiority of the combination regimen was statistically significant (See the below table). Similarly, while 5 of 10 DEX treated mice (50%) and 12 of 20 RJX treated mice (60%) had severe residual liver damage, only 1 of 20 mice (10%) treated with the DEX+RJX combination had severe residual liver damage and this difference was statistically significant (See the below table).

| | Lung Histopathological Score: ≥3 | | |
|---|---|---|---|
| | (severe damage or more) | | |
| | | | LPS-GaIN + |
| | LPS-GaIN + DEX | LPS-GaIN + RJX | DEX + RJX |
| | 5/10 (50.0%) | 14/20 (70.0%) | 2/20 (10.0%) |
| pairwise | | X² | p-value |
| LPS-GaIN + | LPS-GaIN + | 1.148 | 0.425^{a} |
| DEX | RJX | | |
| LPS-GaIN + | LPS-GaIN + | 5.963 | 0.026^{a} |
| DEX | DEX + RJX | | |
| LPS-GaIN + | LPS-GaIN + | 15.000 | 0.0001 |
| RJX | DEX + RJX | | |

| | Liver Histopathological Score: ≥3 | | |
|---|---|---|---|
| | (severe damage or more) | | |
| | | | LPS-GaIN + |
| | LPS-GaIN + DEX | LPS-GaIN + RJX | DEX + RJX |
| | 5/10 (50.0%) | 12/20 (60.0%) | 2/20 (10.0%) |
| pairwise | | X² | p-value |
| LPS-GaIN + | LPS-GaIN + RJX | 0.271 | 0.705^{a} |
| DEX | | | |
| LPS-GaIN + | LPS-GaIN + | 5.963 | 0.026^{a} |
| DEX | DEX + RJX | | |
| LPS-GaIN + | LPS-GaIN + | 10.989 | 0.002 |
| RJX | DEX + RJX | | |

### Example 3.3—Discussion

COVID-19 has become a leading cause of death. Patients with high-risk COVID-19 are in urgent need of effective strategies that can prevent and/or reverse the systemic inflammatory process and its complications, including acute respiratory distress syndrome (ARDS) and multi-organ failure. DEX has been shown to improve the survival outcome of patients with ARDS. Furthermore, in the recently published open-label randomized “RECOVERY” trial, the use of DEX in hospitalized hypoxemic COVID-19 patients requiring invasive mechanical ventilation has been associated with improved survival outcomes. Similar findings were reported from other studies.

The composition, mode of action, and recently published favorable clinical safety profile of RJX make it an attractive anti-inflammatory drug candidate to prevent and treat sepsis.

IL-6, TNF-α, and TGF-β are important pro-inflammatory cytokines that contribute to the pathophysiology of the cytokine release syndrome (CRS), ARDS and multi-organ failure in critically sick adult COVID-19 patients as well as children and adolescents with COVID-19 who develop a multi-system inflammatory syndrome (MIS-C). Recently, it was demonstrated that RJX prevents in the LPS-GalN mouse model of sepsis the marked increase of each of these cytokines in the serum as well as lungs and liver. Here, the combination of RJX plus DEX in therapeutic settings in a preclinical sepsis model was probed. The data provide preclinical proof regarding the clinical impact potential RJX and its combination with DEX to treat sepsis. Treatments with a combination of RJX plus DEX, when initiated after the onset of systemic inflammation and inflammatory organ damage post-injection of an invariably fatal dose of LPS-GalN, immediately reversed the inflammatory cytokine responses, reversed inflammatory organ damage in lungs and liver within 24 hours, and significantly improved survival.

The superiority of the combination regimen was particularly striking when comparing the histopathological data on sepsis-associated organ damage in lungs and liver from mice treated with monotherapy (RJX or DEX alone) vs. combination therapy. Notably, initiation of combination therapy after onset of the inflammatory cytokine responses and systemic inflammation resulted within 24 hours in a near-complete recovery of very severe organ damage in the lungs that was caused by LPS-GalN induced sepsis. It was hypothesized that RJX plus DEX will shorten the time to resolution of lung injury and viral sepsis in COVID-19 patients by preventing the development of a fulminant cytokine storm as well as reversing the cytokine-mediated multi-system inflammatory process and thereby mitigating the inflammatory organ injury. Furthermore, the demonstrated prevention of TGF-β production in the lungs in RJX-treated mice, it was postulated that RJX alone or in combination with DEX may also help reduce the risk of pulmonary fibrosis after ARDS. RJX is currently being evaluated in hospitalized COVID-19 patients with viral sepsis to test the hypothesis that it will contribute to a faster resolution of respiratory failure and a reduced case mortality rate. A study has also been designed to determine if RJX plus DEX combination can reduce the mortality rate of severe to critical COVID-19.

### Example 3.4—Materials and Methods

### Example 3.4.1—LPS-GalN Model of Fatal Cytokine Storm and Sepsis

The anti-sepsis activities of RJX, DEX, and RJX plus DEX were evaluated in the LPS-GalN model of fatal sepsis, as previously described. The research project was approved by the Animal Care and Use Committee of Firat University. Male BALB/c mice were randomly divided into different treatment groups using a pseudo-randomization convenience allocation to assign mice to identified cages. As in previous studies, we applied the concealment of treatment allocation and blind outcome assessment to reduce the risk of bias.

In order to induce fatal sepsis, mice were challenged with an i.p. injection of LPS plus D-galactosamine (Sigma, St. Louis, MO). Each mouse received a 500 μL i.p. injection of LPS-GalN (consisting of 100 ng of LPS plus 8 mg of D-galactosamine). Treatments were delayed until 2 hours post LPS-GalN injection when mice have a fulminant systemic inflammation with very severe lung and liver damage as well as markedly elevated inflammatory cytokine levels. Vehicle control mice were treated with 0.5 mL NS instead of RJX. NS was administered i.p 2 hours after LPS-GalN. Test mice received either RJX (0.7 ml/kg or 1.4 ml/kg) or DEX (0.1 mg/kg, 0.6 mg/kg or 6.0 mg/kg) as a monotherapy in a side-by-side comparison. Also examined was a combination of 0.7 mL/kg RJX with either 0.6 mg/kg or 6 mg/kg DEX at 2 hours post LPS-GalN injection. Drugs were administered i.p. in a total volume of 0.5 ml. The human equivalent dose (HED) level were determined as described. Mice were monitored for mortality for 24 h. The Kaplan-Meier method, log-rank X² test, was used to analyze the 24 h survival outcomes of mice in the different treatment groups. At the time of death, lungs and liver were harvested, fixed in 10% buffered formalin, and processed for histopathologic examination. 3 μm sections were cut, deparaffinized, dehydrated, and stained with haematoxylin and eosin (H & E) and examined with light microscopy. Blood samples were collected at the time of death or termination and used for measurement of inflammatory cytokine and LDH levels, as reported.

### Example 3.4.2—Statistical Analysis

Statistical analyses employed standard methods, including analysis of variance (ANOVA) and/or, nonparametric analysis of variance (Kruskal-Wallis) using the SPSS statistical program (IBM, SPPS Version 21), as reported. Kaplan-Meier method, log-rank X²-test, was used to investigate survival and fatality in each group.

### Example 4.1—Treatment of Patients with COVID-19

Patients that are at risk of developing ARDS may be treated by a method herein. This example outlines treatment of 6 hospitalized adult patients (Age: 24-67 years) with high-risk COVID-19 who were at very high risk for development of hypoxemic respiratory failure and ARDS with a combination of RJX and Dexamethasone that was part of standard of care (in one patient Solumedrol was used instead of Dexamethasone as per the institutional standard of care).

Standard of care also included anti-coagulants, broad-spectrum antibiotics, remdesivir, and in one patient convalescent plasma. Each patient was on oxygen therapy because of a multi-focal bilateral COVID-19 pneomonia. Each patient had highly elevated serum markers for inflammation. RJX was used at a daily flat dose of 20 ml mixed with 100 mL normal saline (total volume of unfudate=120 mL). It was administered intravenously over 40 min. The weight of the patients ranged from 65.8 kg to 156 kg (65.8 kg, 90.7 kg, 102.8 kg, 105 kg, 118 kg, 156 kg; Median=104 kg). Hence, the dose of RJX varied from 0.3 mL/kg to 0.1 mL/kg.

Each of the 6 patients showed rapid recovery and were discharged from hospital within 3-7 days; no patient had to be re-admitted or experienced a worsening of their conditioning following their discharge. Patients were treated only while hospitalized and therefore they received 3-7 doses of RJX.

### Example 4.2—Treatment of Critically Ill COVID-19 Patients with Hypoxemic Failure Who were Hospitalized and Receiving High Flow Oxygen and/or Non-Invasive Positive Pressure Ventilation

Three adult critically ill hospitalized COVID-19 patients with ages of 43 years, 60 years, and 70 years with hypoxemic failure due to advanced COVID-19 pneumonia and viral sepsis were treated with a combination of RJX and Dexamethasone that was part of standard of care. Standard of care also included anti-coagulants, broad-spectrum antibiotics, remdesivir.

Each patient was on high flow oxygen therapy and positive pressure ventilation because of a multi-focal bilateral COVID-19 pneomonia. Each patient had highly elevated serum markers for inflammation. RJX was used at a daily flat dose of 20 ml. It was administered intravenously over 40 min.

The weight of the patients ranged from 66.7 kg to 89.4 kg (66.7 kg, 77 kg, 89.4 kg; Median=77 kg). Hence, the dose of RJX varied from 0.3 mL/kg to 0.2 mL/kg and it was administered in a volume of 120 mL.

Each of these patients showed rapid recovery and were discharged from hospital within 7-14 days; no patient had to be re-admitted or experienced a worsening of their conditioning following their discharge. Patients were treated only while hospitalized and therefore they received 7 doses of RJX. Hence, treatment with RJX in combination with Dexamethasone-including standard of care reversed the viral sepsis.

Two lots of RJX were used: The percentages of ascorbic acid, thiamine HCl, cyanocobalamin, niacinamide, pyridoxin HCl, riboflavin 5′-Phosphate, calcium pantothenate, and magnesium sulfate were 8.981%, 0.625%, 0.019%, 1.193%, 1.200%, 0.025%, 0.028%, and 8.151% in Lot PPP-18-1031 and 9.011%, 0.643%, 0.019%, 1.186%, 1.191%, 0.025%, 0.028%, and 8.176% in Lot PPP-18-1051.

### Example 5—: RJX Promotes Wound Healing

Studies were conducted in diabetic rats on a high-fat diet.

See Pin-Chun Chao, Yingxiao Li, Chin-Hong Chang, Ja Ping Shieh, Juei-Tang Cheng, Kai-Chun Cheng Investigation of insulin resistance in the popularly used four rat models of type-2 diabetesBiomed Pharmacother. 2018 May; 101:155-161. doi: 10.1016/j.biopha.2018.02.084; and Celani L M S, Lopes I S, Medeiros A C. The effect of hyaluronic acid on the skin healing in rats. J Surg Cl Res 10 (2) 2019: 65-75. doi: https://doi.org/10.20398/jscr.v10 i2.18824.

Study Design (N=100):

Wistar albino rats were randomly assigned to control (n=20) and diabetic wound groups (n=80). The rats in the control group underwent full-thickness skin resection in the back (size: 5 mm diameter, with depth to the fascial layer) and be treated by physiological saline, i.p. The rats in the diabetic wound groups were fed a high-fat diet for 4 weeks and then intraperitoneally injected with STZ (45 mg/kg) after being fasted for 16 h. The rat with fasting blood glucose 13.88 mmol/L or 250 mg/dl was considered as a successful model of diabetes (Chao et al., 2018).

After one week, a wound was made in the rat back (size and depth were the same as in the control group). These rats were further randomly assigned to 3 treatment groups (n=20/group; Table 2): (1) diabetic group (DM): induced diabetes, without RJX 0.5 ml/day i.p. normal saline as vehicle; (2) DM+RJX low group (DM+RJX-low): induced diabetes, with RJX- low dose (1.25 mL/kg/day RJX-P, i.p); 3) DM+RJX high group (DM+RJX-high): induced diabetes, with RJX-high dose (2.5 mL/kg/day RJX-B, i.p).

In diabetic group 1, vehicle control rats were treated with 0.5 mL normal saline (NS), i.e., an aqueous solution of 0.9% NaCl instead of RJX. NS (2.5 mL/kg/day) was administered intraperitoneally (i.p). On days 3, 7, 14, and 21, five rats in each group were randomly selected and terminated.

Study Design Table

| | | Treatments | | | |
|---|---|---|---|---|---|
| | | | NS | | |
| No | Gruplar | HFD + STZ | (i.p) | RJX 1.25 i.p | RJX 2.5 i.p |
| 1 | Control | − | + | − | − |
| 2 | DM + NS | + | + | − | − |
| 3 | DM + RJX 1.25 | + | − | + | − |
| 4 | DM + RJX 2.5 | + | − | − | + |

DM: Diabetes;
HFD: High Fat-diet;
STZ: Streptozotocin;
NS: Normal Saline;
RJX: Rejuvenix

### Specimen Collection and Processing

Wound healing was dynamically observed to calculate the healing rate. Wound healing rate=(original area-residual area)/original area×100%. On days 3, 7, 14, and 21, five rats in each group were randomly selected and were injected intramuscular injection of 85 mg/kg ketamine hydrochloride (Ketalar, Pfizer) and 6 mg/kg xylazine hydrochloride (Rompun, Bayer) anesthesia. Wound tissues in the model area were collected to conduct the pathological examination and biological examination.

### Qualitative Examination of the Wound and Wound Area Measurements

The wound borders were marked on a transparent plastic film with a fine-tipped marker. The wound areas were measured by millimetric paper and planimeter. Beginning on the day wound contraction began, each examination measured the wound contraction rate, the fraction of the wound healed, and the expansion rate. Then, the day epithelialization has first noticed, the fraction of wound healed by epithelialization and the number of days it took for healing to occur were documented. Wound sizes wound area and wound contraction were determined.

### Histopathological Examination

The full-thickness wounded skin tissue samples, including the adjacent skin, were photographed and then removed after sacrificing the rats on 3, 7, 14, and 21 days of treatment and subjected to histopathological examination. The tissue samples were fixed in 10% neutral-buffered formalin solution, embedded in paraffin wax, cut into 5-μm-thick sections made from the center of each wound, and stained with hematoxylin-eosin and Masson's trichrome stain, and examined by light microscopy. The histological scoring was assigned in a blinded manner as described previously (Table 3) (Celani et al., 2019).

Histopathological Score for Wound Healing Table

| | | Granulation | Collagen |
|---|---|---|---|
| Score | Re-epithelialization | tissue formation | organization |
| 0 | None | None | None |
| 1 | Migrating | Hypo cellular with few | Trace |
| | | vessels | |
| 2 | Partial stratum | Many vessels and some cells | Slight |
| | corneum | | |
| 3 | Hypertrophic | Many fibroblasts, some fibers | Moderate |
| 4 | Complete and | More fibers few cells | Marked |
| | normal | | |

### Statistical Analysis

Statistical analysis includes analysis of variance (ANOVA) and/or, nonparametric analysis of variance (Kruskall-Wallis) using the statistical programs (IBM, SPPS Version 21 or/and GraphPad Prism version 8.0). Shapiro-Wilk test was performed for normality with a significance level of 0.05. If the data shows normally distribution (Shapiro-Wilk test result p 0.05), a parametric analysis of variance (ANOVA) was performed. After that, a parametric analysis of variance (ANOVA) was performed and Tukey's multiple comparisons were used as a post hoc test to detect alterations among the groups. Independent samples T-test was used as pairwise comparisons for normally distribution two groups. If the ANOVA results were “not significantly different” (F test p >0.05), the analysis stopped. If the data did not show normally distribution (Shapiro-Wilk test result p<0.05), a non-parametric analysis of variance (Kruskal-Wallis) was performed. If the Kruskal-Wallis test returned “statistically different” (p<0.05), the Dunn's multiple corporation test or Mann Whitney U test were performed to compare the values of the treatment groups to those of the vehicle/control group(s). If the Kruskal-Wallis test returned “not significantly different” (p 0.05), the analysis stopped. p-values <0.05 were considered significant.

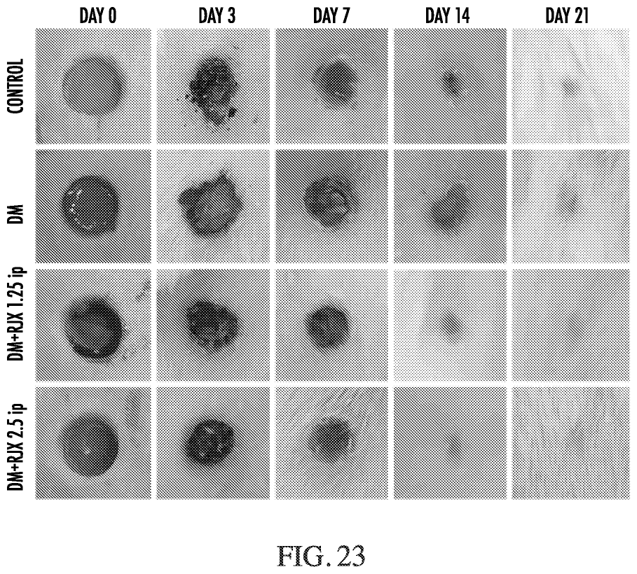

Referring FIG. 23, the effects of Rejuveinix (RJX) on macroscopic changes in diabetic wound healing are illustrated. Groups of 20 Wistar albino rats were treated with i.p injections of RJX (1.25 mL/kg and/or 2.5 mL/kg), or vehicle (NS). Except for untreated control rats (Control), each rat was fed a high-fat diet (HFD) for 4 weeks and injected a single dose of streptozotocin (STZ, 45 mg/kg i.p.) to induced diabetes (DM). At the end of 4 weeks, an experimental wound with a diameter of 5 mm was formed in all rats. On days 3, 7, 14, and 21, five rats in each group were randomly selected. DM: No RJX was used. Instead these rats were treated with 2.5 mL/kg NS daily.

Compared with healthy rats used as controls, diabetic rats receiving NS as treatment (DM+NS) showed significantly delayed would healing as documented by significantly larger residual wound areas at the time points 7 days, 14 days and 21 days. Notably, treatment of diabetic rats with RJX significantly accelerated the wound healing in a dose-dependent manner. The wound healing in diabetic rats treated with 2.5 mL/kg RJX was even faster than the wound healing in untreated healthy rats.

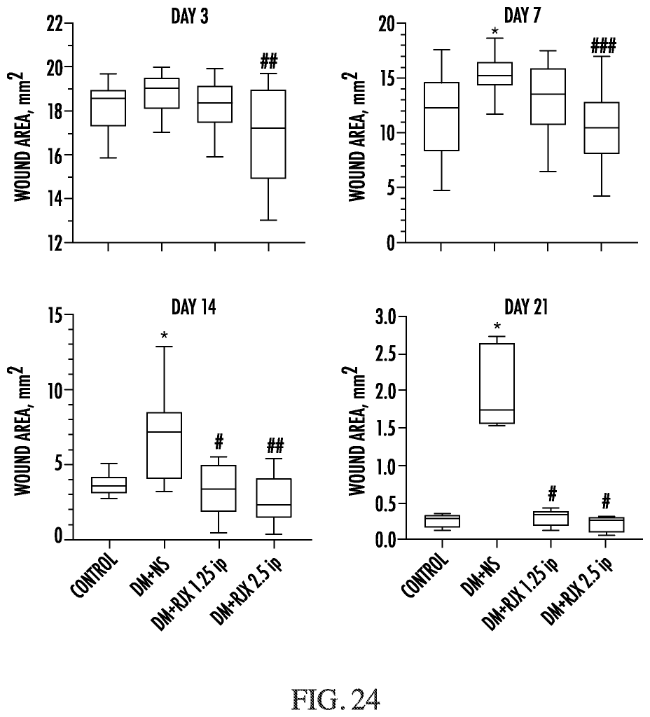

Referring to FIG. 24, the effects of Rejuveinix (RJX) on wound area in diabetic wound healing is illustrated. Groups of 20 Wistar albino rats were treated with i.p injections of RJX (1.25 mL/kg and/or 2.5 mL/kg), or vehicle (NS). Except for untreated control rats (Control), each rat was fed a high-fat diet (HFD) for four weeks and injected a single dose of streptozotocin (STZ, 45 mg/kg i.p.) to induced diabetes (DM). At the end of 4 weeks, an experimental wound with a diameter of 5 mm (initial wound areas=19.625 mm²) was formed in all rats. The depicted wound area data represent the median and min-max. Statistical significance between groups is shown by * p<0.05 as compared to control group, and #p<0.05; ##p<0.01 as compared to DM+NS group. Welch-ANOVA and Tamhane T2 post-hoc test were used for comparing the results among different treatment groups.

In agreement with the observed microscopic wound healing data, the histopathological wound healing scores based on microscopic evaluations showed marked differences between NS treated vs. RJX treated diabetic rats, especially on days 7 and 14 confirming the accelerated wound healing with RJX treatment. Levels of re-epithelialization, granulation tissue formation, and collagen organization were higher in the rats of RJX-treated (particularly RJX 2.5 ip) groups compared to the diabetic control group treated with NS instead of RJX.

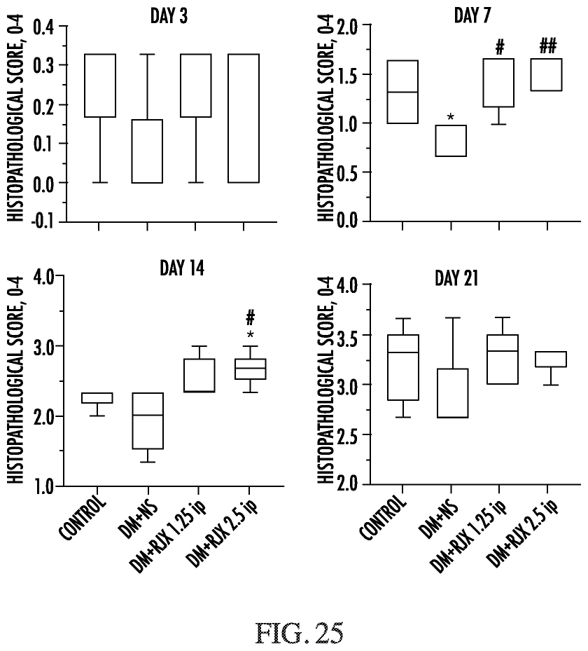

Referring to FIG. 25, the effects of Rejuveinix (RJX) on histopathological score of wounds in diabetic wound healing is illustrated. Groups of 20 Wistar albino rats were treated with i.p injections of RJX (1.25 mL/kg and/or 2.5 mL/kg), or vehicle (NS). Except for untreated control rats (Control), each rat was fed a high-fat diet (HFD) for 4 weeks and injected a single dose of streptozotocin (STZ, 45 mg/kg i.p.) to induced diabetes (DM). At the end of 4 weeks, an experimental wound with a diameter of 5 mm was formed in all rats. On days 3, 7, 14, and 21, five rats in each group were randomly selected. The depicted wound area data represent the median and min-max. Statistical significance between groups is shown by * p<0.05 as compared to control group, and #p<0.05; ##p<0.01 as compared to DM+NS group. Kruskal Wallis and Mann Whitney U tests were used for comparing the results among different treatment groups.

Mouse doses in the foregoing examples may be converted to human doses for embodiments herein by a conversion factor of ˜12.

### REFERENCES

Kadri, S. S. et al. Estimating Ten-Year Trends in Septic Shock Incidence and Mortality in United States Academic Medical Centers Using Clinical Data. Chest 151, 278-285 (2017).
Rhee, C. et al. Incidence and Trends of Sepsis in US Hospitals Using Clinical vs Claims Data, 2009-2014. JAMA 318, 1241 (2017).
Rudd, K. E. et al. Global, regional, and national sepsis incidence and mortality, 1990-2017: analysis for the Global Burden of Disease Study. Lancet 395, 200-211 (2020).
Anand, V., Zhang, Z., Kadri, S. S., Klompas, M. & Rhee, C. Epidemiology of Quick Sequential Organ Failure Assessment Criteria in Undifferentiated Patients and Association With Suspected Infection and Sepsis. Chest 156, 289-297 (2019).
Rhodes, A. et al. Surviving Sepsis Campaign. Crit. Care Med. 45, 486-552 (2017).
Kaukonen, K.-M., Bailey, M., Pilcher, D., Cooper, D. J. & Bellomo, R. Systemic Inflammatory Response Syndrome Criteria in Defining Severe Sepsis. N. Engl. J. Med. 372, 1629-1638 (2015).
Meyer, N. et al. Temporal trends in incidence, sepsis-related mortality, and hospital-based acute care after sepsis. Crit. Care Med. 46, 354-360 (2018).
Seymour, C. W. et al. Derivation, Validation, and Potential Treatment Implications of Novel Clinical Phenotypes for Sepsis. JAMA 321, 2003 (2019).
Li, H. et al. SARS-CoV-2 and viral sepsis: observations and hypotheses. Lancet 395, 1517-1520 (2020).
Uckun, F. M. Reducing the Fatality Rate of COVID-19 by Applying Clinical Insights From Immuno-Oncology and Lung Transplantation. Front. Pharmacol. 11, 796 (2020).
Wu, C. et al. Risk Factors Associated With Acute Respiratory Distress Syndrome and Death in Patients With Coronavirus Disease 2019 Pneumonia in Wuhan, China. JAMA Intern. Med. 180, 934 (2020).
Lian, J. et al. Analysis of Epidemiological and Clinical Features in Older Patients With Coronavirus Disease 2019 (COVID-19) Outside Wuhan. Clin. Infect. Dis. 71, 740-747 (2020).
Zhou, F. et al. Clinical course and risk factors for mortality of adult inpatients with COVID-19 in Wuhan, China: a retrospective cohort study. Lancet 395, 1054-1062 (2020).
Catherine, C. & Jones, N. Association of Inpatient Use of Angiotensin-Converting Enzyme Inhibitors and Angiotensin II Receptor Blockers with Mortality among Patients with Hypertension Hospitalized with COVID-19. Circulation Research vol. 126 1671-1681 (2020).
Zheng, Z. et al. Risk factors of critical &amp; mortal COVID-19 cases: A systematic literature review and meta-analysis. J. Infect. 81, e16-e25 (2020).
Kuderer, N. M. et al. Clinical impact of COVID-19 on patients with cancer (CCC19): a cohort study. Lancet 395, 1907-1918 (2020).
Uckun, F. M. et al. Rejuveinix Shows a Favorable Clinical Safety Profile in Human Subjects and Exhibits Potent Preclinical Protective Activity in the Lipopolysaccharide-Galactosamine Mouse Model of Acute Respiratory Distress Syndrome and Multi-Organ Failure. Front. Pharmacol. 11, (2020).
Nair, A. & Jacob, S. A simple practice guide for dose conversion between animals and human. J. Basic Clin. Pharm. 7, 27 (2016).
Woolf, S. H., Chapman, D. A. & Lee, J. H. COVID-19 as the Leading Cause of Death in the United States. JAMA—Journal of the American Medical Association vol. 325 123-124 (2021).
Faust, J. S. et al. All-Cause Excess Mortality and COVID-19-Related Mortality Among US Adults Aged 25-44 Years, March-July 2020. JAMA 325, 785 (2021).
Woolf, S. H. et al. Excess Deaths From COVID-19 and Other Causes, March-July 2020. JAMA 324, 1562 (2020).
Villar, J. et al. Dexamethasone treatment for the acute respiratory distress syndrome: a multicentre, randomised controlled trial. Lancet Respir. Med. 8, 267-276 (2020).
The RECOVERY Collaborative Group. Dexamethasone in Hospitalized Patients with Covid-19. N. Engl. J. Med. 384, 693-704 (2021).
Sterne, J. A. C. et al. Association Between Administration of Systemic Corticosteroids and Mortality Among Critically III Patients With COVID-19. JAMA 324, 1330 (2020).
Tomazini, B. M. et al. Effect of Dexamethasone on Days Alive and Ventilator-Free in Patients With Moderate or Severe Acute Respiratory Distress Syndrome and COVID-19. JAMA 324, 1307 (2020).
Li, J. Evidence is stronger than you think: a meta-analysis of vitamin C use in patients with sepsis. Crit. Care 22, 258 (2018).
Putzu, A., Daems, A.-M., Lopez-Delgado, J. C., Giordano, V. F. & Landoni, G. The Effect of Vitamin C on Clinical Outcome in Critically III Patients. Crit. Care Med. 47, 774-783 (2019).
Obi, J., Pastores, S. M., Ramanathan, L. V., Yang, J. & Halpern, N. A. Treating sepsis with vitamin C, thiamine, and hydrocortisone: Exploring the quest for the magic elixir. J. Crit. Care 57, 231-239 (2020).
Hager, D. N. et al. The Vitamin C, Thiamine and Steroids in Sepsis (VICTAS) Protocol: a prospective, multi-center, double-blind, adaptive sample size, randomized, placebo-controlled, clinical trial. Trials 20, 197 (2019).
Iglesias, J. et al. Outcomes of Metabolic Resuscitation Using Ascorbic Acid, Thiamine, and Glucocorticoids in the Early Treatment of Sepsis. Chest 158, 164-173 (2020).
Shan, M. et al. Vitamin B6 inhibits macrophage activation to prevent lipopolysaccharide-induced acute pneumonia in mice. J. Cell. Mol. Med. 24, 3139-3148 (2020).
Moskowitz, A. et al. Effect of Ascorbic Acid, Corticosteroids, and Thiamine on Organ Injury in Septic Shock. JAMA 324, 642 (2020).
Fujii, T. et al. Effect of Vitamin C, Hydrocortisone, and Thiamine vs Hydrocortisone Alone on Time Alive and Free of Vasopressor Support Among Patients With Septic Shock. JAMA 323, 423 (2020).
Byerly, S. et al. Vitamin C and thiamine are associated with lower mortality in sepsis. J. Trauma Acute Care Surg. 89, 111-117 (2020).
Zumla, A., Hui, D. S., Azhar, E. I., Memish, Z. A. & Maeurer, M. Reducing mortality from 2019-nCoV: host-directed therapies should be an option. Lancet 395, e35-e36 (2020).
Cabala, K., Earabino, J., Pacult, P. & Uckun, F. M. Rationale for a randomized, placebo-controlled, Phase 2 study of Rejuveinix (RJX) in COVID-19 patients with acute lung injury and hypoxemic respiratory failure. Clin. Investig. (Lond). 10, 62-66 (2020).
Frank, J. A. & Matthay, M. A. TGF- and lung fluid balance in ARDS. Proc. Natl. Acad. Sci. 111, 885-886 (2014).
Peters, D. M. et al. TGF—directs trafficking of the epithelial sodium channel ENaC which has implications for ion and fluid transport in acute lung injury. Proc. Natl. Acad. Sci. 111, E374-E383 (2014).
Hu, X. & Huang, X. Alleviation of Inflammatory Response of Pulmonary Fibrosis in Acute Respiratory Distress Syndrome by Puerarin via Transforming Growth Factor (TGF-β1l). Med. Sci. Monit. 25, 6523-6531 (2019).
Shimbori, C. et al. Fibroblast growth factor-1 attenuates TGF-β1-induced lung fibrosis. J. Pathol. 240, 197-210 (2016).
Dufort, E. M. et al. Multisystem Inflammatory Syndrome in Children in New York State.
N. Engl. J. Med. 383, 347-358 (2020).
Feldstein, L. R. et al. Multisystem Inflammatory Syndrome in U.S. Children and Adolescents. N. Engl. J. Med. 383, 334-346 (2020).
Riphagen, S., Gomez, X., Gonzalez-Martinez, C., Wilkinson, N. & Theocharis, P. Hyperinflammatory shock in children during COVID-19 pandemic. Lancet 395, 1607-1608 (2020).
Davies, P. et al. Intensive care admissions of children with paediatric inflammatory multisystem syndrome temporally associated with SARS-CoV-2 (PIMS-TS) in the UK: a multicentre observational study. Lancet Child Adolesc. Heal. 4, 669-677 (2020).
McCrindle B W, Rowley A H, Newburger J W, et al. Diagnosis, Treatment, and Long-Term Management of Kawasaki Disease: A Scientific Statement for Health Professionals From the American Heart Association Circulation. 2017; 135(17):e927-e999.
Newburger J W, Takahashi M, Gerber M A, et al. Diagnosis, treatment, and long-term management of Kawasaki disease: a statement for health professionals from the Committee on Rheumatic Fever, Endocarditis, and Kawasaki Disease, Council on Cardiovascular Disease in the Young, American Heart Association. Pediatrics. 2004; 114(6): 1708-1733.
American Academy of Pediatrics. Kawasaki disease. In: Red Book: 2018 Report of the Committee on Infectious Diseases, 31st ed, Kimberlin D W, Brady M T, Jackson M A, Long S S (Eds), American Academy of Pediatrics, Itasca, IL 2018 p.490.
Stavnezer J. Regulation of antibody production and class switching by TGF-beta. J Immunol. 1995; 155(4): 1647-1651.
Yim D, Ramsay J, Kothari D, Burgner D. Coronary artery dilatation in toxic shock-like syndrome: the Kawasaki disease shock syndrome. Pediatr Cardiol. 2010; 31(8): 1232-1235.
Gatterre P, Oualha M, Dupic L, et al. Kawasaki disease: an unexpected etiology of shock and multiple organ dysfunction syndrome. Intensive Care Med. 2012; 38(5): 872-878.
Palazzi D L, McClain K L, Kaplan S L. Hemophagocytic syndrome after Kawasaki disease. Pediatr Infect Dis J. 2003; 22(7): 663-666.
Latino G A, Manlhiot C, Yeung R S, Chahal N, McCrindle B W. Macrophage activation syndrome in the acute phase of Kawasaki disease. J Pediatr Hematol Oncol. 2010; 32(7): 527-531.
Hendricks M, Pillay S, Davidson A, De Decker R, Lawrenson J. Kawasaki disease preceding haemophagocytic lymphohistiocytosis: challenges for developing world practitioners. Pediatr Blood Cancer. 2010; 54(7): 1023-1025.
Royal College of Paediatrics and Child Health. Guidance: Paediatric multisystem inflammatory syndrome temporally associated with COVID-19. https://www.rcpch.ac.uk/resources/guidance-paediatric-multisystem-inflammatory-syndrome-temporally-associated-covid-19 (Accessed on May 7, 2020).
United States Centers for Disease Control and Prevention. Health Alert Network. Multisystem inflammatory syndrome in children (MIS-C) associated with coronavirus disease 2019 (COVID-19). Available at: https://emergency.cdc.gov/han/2020/han00432.asp (Accessed on May 15, 2020).
Dufort E M, Koumans E H, Chow E J, et al. Multisystem Inflammatory Syndrome in Children in New York State. N Engl J Med. 2020; 383(4): 347-358.
Riphagen S, Gomez X, Gonzalez-Martinez C, Wilkinson N, Theocharis P. Hyperinflammatory shock in children during COVID-19 pandemic. Lancet. 2020; 395(10237): 1607-1608.
European Centre for Disease Prevention and Control Rapid Risk Assessment: Paediatric inflammatory multisystem syndrome and SARS CoV 2 infection in children. Available at: https://www.ecdc.europa.eu/sites/default/files/documents/covid-19-risk-assessment-paediatric-inflammatory-multisystem-syndrome-15May 2020.pdf (Accessed on May 17, 2020).
Verdoni L, Mazza A, Gervasoni A, et al. An outbreak of severe Kawasaki-like disease at the Italian epicentre of the SARS-CoV-2 epidemic: an observational cohort study. Lancet. 2020; 395(10239): 1771-1778.
Feldstein L R, Rose E B, Horwitz S M, et al. Multisystem Inflammatory Syndrome in U.S. Children and Adolescents. N Engl J Med. 2020; 383(4): 334-346.
Godfred-Cato S, Bryant B, Leung J, et al. COVID-19-Associated Multisystem Inflammatory Syndrome in Children—United States, March-July 2020. MMWR Morb Mortal Wkly Rep. 2020; 69(32): 1074-1080.
Belhadjer Z, Meot M, Bajolle F, et al. Acute heart failure in multisystem inflammatory syndrome in children (MIS-C) in the context of global SARS-CoV-2 pandemic. Circulation. 2020; 10.1161/CIRCULATIONAHA.120.048360.
Davies P, Evans C, Kanthimathinathan H K, et al. Intensive care admissions of children with paediatric inflammatory multisystem syndrome temporally associated with SARS-CoV-2 (PIMS-TS) in the UK: a multicentre observational study. Lancet Child Adolesc Health. 2020; 4(9): 669-677.
Center for Disease Control and Prevention, Center for Preparedness and Response: Multisystem Inflammatory Syndrome in Children (MIS-C) Associated with Coronavirus Disease 2019 (COVID-19), Clinician Outreach and Communication (COCA) Webinar. Available at: https://emergency.cdc.gov/coca/calls/2020/callinfo_051920.asp?deliveryName=USCDC_1052-DM28623 (Accessed on May 19, 2020).
Sperotto F, Friedman K G, Son M B F, VanderPluym C J, Newburger J W, Dionne A. Cardiac manifestations in SARS-CoV-2-associated multisystem inflammatory syndrome in children: a comprehensive review and proposed clinical approach. Eur J Pediatr. 2020; 1-16.
Kaushik A, Gupta S, Sood M, Sharma S, Verma S. A Systematic Review of Multisystem Inflammatory Syndrome in Children Associated With SARS-CoV-2 Infection [published online ahead of print, 2020 Sep. 8]. Pediatr Infect Dis J. 2020; 10.1097/INF.0000000000002888.
Radia T, Williams N, Agrawal P, et al. Multi-system inflammatory syndrome in children & adolescents (MIS-C): A systematic review of clinical features and presentation [published online ahead of print, 2020 Aug. 11]. Paediatr Respir Rev. 2020; S1526-0542(20)30117-2.
Whittaker E, Bamford A, Kenny J, et al. Clinical Characteristics of 58 Children With a Pediatric Inflammatory Multisystem Syndrome Temporally Associated With SARS-CoV-2. JAMA. 2020; 324(3): 259-269.
Uckun F M. Reducing the Fatality Rate of COVID-19 by Applying Clinical Insights From Immuno-Oncology and Lung Transplantation. Front Pharmacol. 2020; 11:796.
Huang C, Wang Y, Li X, et al. Clinical features of patients infected with 2019 novel coronavirus in Wuhan, China. Lancet. 2020; 395(10223): 497-506.
Young B E, Ong S W X, Kalimuddin S, et al. Epidemiologic Features and Clinical Course of Patients Infected With SARS-CoV-2 in Singapore. JAMA. 2020; 323(15): 1488-1494.
Zumla A, Hui D S., Azhar E I, Memish Z A, Mauerer M. Reducing mortality from 2019-nCoV: host-directed therapies should be an option. Lancet. 2020; 395(10224):e35-e36. doi: 10.1016/S0140-6736(20)30305-6.
Chen X, Zhao B, Qu Y, Chen Y, Xiong J, Feng Y, Men D, Huang Q, Liu Y, Yang B, Ding J, Li F. Detectable serum SARS-CoV-2 viral load (RNAaemia) is closely correlated with drastically elevated interleukin 6 (IL-6) level in critically ill COVID-19 patients. Clin Infect Dis. 2020 Apr. 17.
Uckun F M, Ervin J, Jim Ervin, et al. Clinical Impact Potential Of Rejuveinix (RJX) For Prevention Of Fatal Acute Respiratory Distress Syndrome (ARDS) And Multi-Organ Failure In Covid-19 Patients. Clinical Investigation (London). 2020; 10(2): 177-184.
Xiong Y, Liu Y, Cao L, et al. Transcriptomic characteristics of bronchoalveolar lavage fluid and peripheral blood mononuclear cells in COVID-19 patients. Emerg Microbes Infect. 2020; 9(1): 761-770.
Uckun F M. Treating Severe COVID-19 in Cancer Patients. Cancer Resports and Reviews 2020; 3:1-2; doi: 10.15761/CRR.1000198.
Uckun F M, Trieu V. Targeting T G F-β for Treatment of COVID-19 Associated Kawasaki Disease in Children. Clin Res Pediatrics. 2020; 3(1): 1-3.
Stafford N, Arnold A, Jebakumar S, Manglam V, Sangwaiya A, Arnold J. Therapeutic strategies for COVID-19: New Insights Into The Value Of Transforming Growth Factor Beta (TGFβ) Antagonists Such As Imatinib and other Kinase Inhibitors. BMJ 2020; 369 doi: https://doi.org/10.1136/bmj.m1610.
Frank J, Roux J, Kawakatsu H, et al. Transforming growth factor-beta1 decreases expression of the epithelial sodium channel alphaENaC and alveolar epithelial vectorial sodium and fluid transport via an ERK1/2-dependent mechanism. J Biol Chem. 2003; 278(45): 43939-43950.
Fahy R J, Lichtenberger F, McKeegan C B, Nuovo G J, Marsh C B, Wewers M D. The acute respiratory distress syndrome: a role for transforming growth factor-beta 1. Am J Respir Cell Mol Biol. 2003; 28(4): 499-503.
Willis B C Kim K J, Li X, Liebler J, Crandall E D, Borok Z. Modulation of ion conductance and active transport by TGF-beta 1 in alveolar epithelial cell monolayers. Am J Physiol Lung Cell Mol Physiol. 2003; 285(6):L1192-L1200.
Jenkins R G, Su X, Su G, et al. Ligation of protease-activated receptor 1 enhances alpha(v)beta6 integrin-dependent TGF-beta activation and promotes acute lung injury. J Clin Invest. 2006; 116(6): 1606-1614.
Pittet J F, Griffiths M J, Geiser T, et al. TGF-beta is a critical mediator of acute lung injury. J Clin Invest. 2001; 107(12): 1537-1544.
Frank J A, Matthay M A. TGF-β and lung fluid balance in ARDS. Proc Natl Acad Sci USA. 2014; 111(3): 885-886.
Peters D M, Vadesz I, Wujak L, et al. TGF-β directs trafficking of the epithelial sodium channel ENaC which has implications for ion and fluid transport in acute lung injury. Proc Natl Acad Sci USA. 2014; 111(3):E374-E383.
Budinger G R, Chandel N S, Donnelly H K, Eisenbart J, Oberoi M, Jain M. Active transforming growth factor-beta1 activates the procollagen I promoter in patients with acute lung injury. Intensive Care Med. 2005; 31(1): 121-128.
Woolum J A, Abner E L, Kelly A, Thompson-Bastin M L, Morris P E, Flannery A H. Effect of Thiamine Administration on Lactate Clearance and Mortality in Patients With Septic Shock. Crit Care Med. 2018; 46(11): 1747-1752.
Fox R, Akmatbekov A, Harbert J, Li G, Brown Q, Vander Heide R S. Pulmonary and cardiac Pathology in COVID —19; the first autopsy series from New Orleans. medRxiv. 2020. Doi: 10.1101/2020.04.06.2
Schönrich G, Raftery M J, Samstag Y. Devilishly radical NETwork in COVID-19: Oxidative stress, neutrophil extracellular traps (NETs), and T cell suppression. Adv Biol Regul. 2020; 77:100741.
Shah M S, Brownlee M. Molecular and Cellular Mechanisms of Cardiovascular Disorders in Diabetes. Circ Res. 2016 May 27; 118(11): 1808-29.
Li S W, Wang C Y, Jou Y J, et al. SARS coronavirus papain-like protease induces Egr-1-dependent up-regulation of TGF-β1 via ROS/p38 MAPK/STAT3 pathway. Sci Rep. 2016; 6:25754.
Liang J, Ziegler J D, Jahraus B, et al. Piperlongumine Acts as an Immunosuppressant by Exerting Prooxidative Effects in Human T Cells Resulting in Diminished TH17 but Enhanced Treg Differentiation. Front Immunol. 2020; 11:1172.
Tan M, Liu Y, Zhou R, et al. Immunopathological characteristics of coronavirus disease 2019 cases in Guangzhou, China. Immunology. 2020; 160(3): 261-268.
Uckun F M, Carlson J, Orhan C, et al. Rejuveinix shows a favorable clinical safety profile in human subjects and exhibit potent preclinical protective activity in the LPS-GalN mouse model of acute respiratory distress syndrome (ARDS). Front. Pharmacol. (Under Rev.)
Van Wyk H, Van Wyk M, Krebs T, Lange P, Denomme B, Volk M A. U.S. Pat. No. 9,775,798 2017; Composition and methods for the prevention and treatment of cardiovascular diseases—Continuation of U.S. patent application Ser. No. 14/550,677 as a divisional of U.S. patent application Ser. No. 13/055,691, which issued as U.S. Pat. No. 9,101,537. Publication #20170360696.
Wang T, Xia Y, Hao D. The significance of lactic acid in early diagnosis and goal-directed therapy of septic shock patients. Chin Crit Care Med 2014; 26:51-5.
Noormandi A, Khalili H, Mohammadi M, Abdollahi A. Effect of magnesium supplementation on lactate clearance in critically ill patients with severe sepsis: a randomized clinical trial. Eur J Clin Pharmacol. 2020; 76(2): 175-184. Pan J, Peng M, Liao C, Hu X, Wang A, Li X. Relative efficacy and safety of early lactate clearance-guided therapy resuscitation in patients with sepsis: A meta-analysis.
Medicine (Baltimore). 2019; 98(8):e14453.
Li J. Evidence is stronger than you think: a meta-analysis of vitamin C use in patients with sepsis. Crit Care. 2018; 22(1): 258.
Putzu A, Daems A M, Lopez-Delgado J C, Giordano V F, Landoni G. The Effect of Vitamin C on Clinical Outcome in Critically Ill Patients: A Systematic Review With Meta-Analysis of Randomized Controlled Trials. Crit Care Med. 2019; 47(6): 774-783.
Obi J, Pastores S M, Ramanathan L V, Yang J, Halpern N A. Treating sepsis with vitamin C, thiamine, and hydrocortisone: Exploring the quest for the magic elixir. J Crit Care. 2020; 57:231-239.
Hager D N, Hooper M H, Bernard G R, et al. The Vitamin C, Thiamine and Steroids in Sepsis (VICTAS) Protocol: a prospective, multi-center, double-blind, adaptive sample size, randomized, placebo-controlled, clinical trial. Trials. 2019; 20(1): 197.
Iglesias J, Vassallo A V, Patel V V, Sullivan J B, Cavanaugh J, Elbaga Y. Outcomes of Metabolic Resuscitation Using Ascorbic Acid, Thiamine, and Glucocorticoids in the Early Treatment of Sepsis: The ORANGES Trial. Chest. 2020; 158(1): 164-173.
Shan M R, Zhou S N, Fu C N, et al. Vitamin B₆ inhibits macrophage activation to prevent lipopolysaccharide-induced acute pneumonia in mice. J Cell Mol Med. 2020; 24(5): 3139-3148.
Moskowitz A, Huang D T, Hou P C, et al. Effect of Ascorbic Acid, Corticosteroids, and Thiamine on Organ Injury in Septic Shock: The ACTS Randomized Clinical Trial. JAMA. 2020; 324(7): 642-650.
Fujii T, Luethi N, Young P J, et al. Effect of Vitamin C, Hydrocortisone, and Thiamine vs Hydrocortisone Alone on Time Alive and Free of Vasopressor Support Among Patients With Septic Shock: The VITAMINS Randomized Clinical Trial.
JAMA. 2020; 323(5): 423-431.
Byerly S, Parreco J P, Soe-Lin H, et al. Vitamin C and thiamine are associated with lower mortality in sepsis. J Trauma Acute Care Surg. 2020; 89(1): 111-117.
Center for Disease Control and Prevention. Health Department-Reported Cases of Multisystem Inflammatory Syndrome in Children (MIS-C) in the United States. 2020. https://www.cdc.gov/mis-c/cases/index.html (Accessed on Sep. 20, 2020).

The references cited throughout this application, are incorporated for all purposes apparent herein and in the references themselves as if each reference was fully set forth. A citation of a reference at a particular location does not limit the manner in which all of the teachings of the cited reference are incorporated for all purposes.

It is understood, therefore, that this invention is not limited to the particular embodiments disclosed, but is intended to cover all modifications which are within the spirit and scope of the invention as defined by the appended claims; the above description; and/or shown in the attached drawings.

1. A pharmaceutical composition for intravenous delivery to a mammal, the pharmaceutical composition comprising magnesium sulfate, ascorbic acid, thiamine, and niacinamide at a ratio (w/w) of 72 to 108 magnesium sulfate: 80 to 120 ascorbic acid: 5.6 to 8.4 thiamine: 10.4 to 15.6 niacinamide, and at least one anti-inflammatory drug selected from anti-inflammatory steroids, or a pharmaceutically acceptable salt thereof.

2. The pharmaceutical composition of claim 1 further comprising pyridoxin and riboflavin, and the magnesium sulfate, ascorbic acid, thiamine, niacinamide, pyridoxin, and riboflavin are at a ratio (w/w) of 72 to 108 magnesium sulfate: 80 to 120 ascorbic acid: 5.6 to 8.4 thiamine: 10.4 to 15.6 niacinamide: 10.4 to 15.6 pyridoxin: 0.24 to 0.36 riboflavin.

3. (canceled)

4. (canceled)

5. The pharmaceutical composition of claim 4, wherein the magnesium sulfate is at a concentration of 0.7 to 0.9 mg/mL, the ascorbic acid is at a concentration of 0.8 to 1.0 mg/mL, the thiamine is at a concentration of 0.05 to 0.07 mg/mL, and the niacinamide is at a concentration of 0.105 to 0.150 mg/mL.

6. The pharmaceutical composition of claim 5, wherein the pyridoxin is at a concentration of 0.105 to 0.150 mg/mL and the riboflavin is at a concentration of 0.002 to 0.003 mg/mL.

7. The pharmaceutical composition of claim 6 further comprising cyanocobalamin.

8. The pharmaceutical composition of claim 7 further comprising at least one of an antioxidant or an anti-inflammatory agent.

9. The pharmaceutical composition of claim 8, wherein the at least one of an antioxidant or an anti-inflammatory agent are selected from Cox-2 or Cox1 inhibitors, steroids, zinc, copper, selenium, Vitamin E, and Vitamin A.

10. The pharmaceutical composition of claim 1, wherein the at least one anti-inflammatory drug selected from anti-inflammatory steroids comprises dexamethasone.

11. The pharmaceutical composition of claim 10, wherein the one or more anti-inflammatory drug selected from anti-inflammatory steroids further comprises one or more selected from cortisone, hydrocortisone, methylprednisolone, prednisolone, prednisone, triamcinolone, fludrocortisone, and betamethasone.

12. The pharmaceutical composition of claim 1, wherein the one or more anti-inflammatory drug selected from anti-inflammatory steroids is selected from cortisone, hydrocortisone, methylprednisolone, prednisolone, prednisone, triamcinolone, betamethasone, fludrocortisone, and dexamethasone.

13. The pharmaceutical composition of claim 12, wherein the one or more anti-inflammatory drug selected from anti-inflammatory steroids comprises dexamethasone at an amount of 0.75-40 mg per dose of the pharmaceutical composition.

14. The pharmaceutical composition of claim 2 further comprising cyanocobalamin, and wherein the magnesium sulfate is at a concentration of 50 to 100× (0.7 to 0.9 mg/mL), the ascorbic acid is at a concentration of 50 to 100×(0.8 to 1.0 mg/mL), the thiamine is at a concentration of 50 to 100×(0.05 to 0.07 mg/mL), the niacinamide is at a concentration of 50 to 100×(0.105 to 0.150 mg/mL), the pyridoxin is at a concentration of 50 to 100×(0.105 to 0.150 mg/mL), the riboflavin is at a concentration of 50 to 100×(0.002 to 0.003 mg/mL), and the cyanocobalamin is at a concentration of 50 to 100×(0.0015 to 0.0030 mg/mL).

15. A method of treating an inflammatory condition in a mammal comprising administering to the mammal an effective amount of a pharmaceutical composition comprising magnesium sulfate, ascorbic acid, thiamine, and niacinamide at a ratio (w/w) of 72 to 108 magnesium sulfate: 80 to 120 ascorbic acid: 5.6 to 8.4 thiamine: 10.4 to 15.6 niacinamide.

16. The method of claim 15 further comprising administering an effective amount of one or more anti-inflammatory drug selected from anti-inflammatory steroids, or a pharmaceutically acceptable salt thereof, to the mammal.

17. The method of claim 15, wherein the one or more anti-inflammatory drug selected from anti-inflammatory steroids comprises dexamethasone.

18. The method of claim 17, wherein the dexamethasone is at a dose of 1-40 mg.

19. The method of claim 17, wherein the pharmaceutical composition further comprises the dexamethasone.

20. The method of claim 15, wherein the mammal is a human.

21.-32. (canceled)

33. A method of treating COVID-19 comprising administering to a COVID-19 patient an effective amount of a pharmaceutical composition comprising magnesium sulfate, ascorbic acid, thiamine, and niacinamide at a ratio (w/w) of 72 to 108 magnesium sulfate: 80 to 120 ascorbic acid: 5.6 to 8.4 thiamine: 10.4 to 15.6 niacinamide, preferably where the administering further comprises administering one or more anti-inflammatory agent, separately or as part of the pharmaceutical composition.

34. The method of claim 33, wherein the one or more anti-inflammatory agent comprises dexamethasone.
